SEQUENCE LISTING (5 sequences)

SEQ ID NO: 1 - DNA, 5000 bp, Rattus norvegicus
caaagcctctggtatcattcataaacttagcaaaaaaaaaaaaaattacaaaaggctttggattttcttagattataagacacagttttgttctaaactgaatttgaatttttaaatatcgacgggtgacaaagaaaaatttaaagactaaaaaataaaaggactgtcacaaggagtattttatggctgctattagaatattaggaagtaagatggggatttggattggagacccgctgcctccccacacacttcaccacctagaatctgagccccaaactttctgtgtcattttaaaaatgaaaatggtagttttgtcttcttctttcttgctttgcaatgtttcccccaaagaaaaattgtggagagcttttggggcttctaggaatttggcaagttttatttaaaaaaaaaaataggagagtgacctgtaacttagaagtgactgtgaaatatgccaccaactggtctactgactggaaatactgggaagcattttcccaggtctgtgaggaaagcacagcttcatttttgcagctattcttttttgcaacatcaaacaattctttaaacgccaaacaactagaaagggctacttccttttatagtgaaacctcaagatctgactccaggtaccaggctagatctagggtgtctacaaagaattcaaaaccgtgcctcttcagcctggaatcccaggagctgaaggttcagggctgggggaagagtgcataggggttgggtacccagacttggccgtacaggttctttgcttctttattttttcttgaataagatgaggtgcgtccaggtgttctcttccaatgtttttgctttgatcagggtgatctcgagatgagggttgatagtcactgaggaaaggaacgtggtgtctgtggtgtcaggctccgttgttaggagaggggaccaaattcgttcagtgcgggtcactttacacatctagctcagatcctttgtagttctatcggcacagctgtacctggggtgtactttcgataggaccaaggtggacttcgtagtgttttcttgtctcccttatcgtacactggaaactgtccaagctccagattctttccagacctcatttctctctcactgctttgcttgtgaattgattcccgctgaattcggagaggcaaccgatgacacagtggtgaccaatgttgatcaccatttaaaccgaatcacatgggtcccagagggcacttgctggtgcccaattccttgggggtggagggaagatctctcttggtctctgtgtgaaatccaacatctgttgtttgttttgtttttccttcgaggaaattcaggtcccaattccttcaagtcccaattcctatgtacgtcgtgttcagcctacagtttcgtttctgctatcttggggataggctttagtctcaggatccttcctggaacaagaaataaaacttactctttttcgatgcaaggattatttacgaataccccttaattcaatcttattatggaaagtcacggacttactgctgggtcattagatgctccacctccaccctgagcaaaaagatctaagaaggttgaaggtgaacctgattttctgcccatagaccatgggaaaggaaagcagaaagggggacgtgagtcgggttgagccaccctcccccggccactcgagtgacatagtccttaaaaatagccaagtctgaacctcagcgtgcccagccccccctcctctctttggctttcctagtctgagaggagcctgggaacctctagaggcagccgataacccgctccatctccagccagcaaggccttgaaatgctccccgctcctggcaacgcacagaggtcgccttggttcctctggttccagcagaagaagggagcaactgaaggaccggaggcaaagggagggagggaatgaagaaaaaaccaaaagcgcccaagaaacacgtctggtttgattctcagcccacacaccgggaaaaaatttgttgtgtgggacccagagtgtgggggttaagtcacaggaagctgaaacaaatgtctctgtcccaggcgccttcggagtcagagaattgtctcgggtctccggctcgcagcagtcagctcagcgggagatccctgcaaagaaaagcggcctgggagtgtttggctgaccttagcaagagctaagaatcacgcgatgcacttacagacttcatagagtctcttaaccgcacagagcaaagtatctccagtctcctttcaaacggctagaacctgggtttcaggaaggatgatgtgtgggtaggggtggggatagttagcttcaggacaccaccttactataaggctcatccgggcacagggagaatgggagaatgcccccctgaaagctgccggagaccttggtaccgcggaggcaggcgttttagagggctgctcccccacctcctccctcagacactaagcctcaagcctcagacttcatgggtgacaaacaaaatcaggaacgctagatttggccccaagcttagtgcattttgattttgatctataggggttactacaacgaggcaggtctgagatgagagagagagagagagagagagagagagagagagagagagagagagagctagccgttggcttcgtttctctcctcaccaggtagctgagggtgtcttgtaaaataaagcaagggccacgccattctctgcattcatccgagagtctcagaaaggagtttgctcgaggagaggtggaaaggagggttcgttgatgcctctggagcccccagaataacgtccctaaacctttaccagacctcgaacctgcagcccctgattctgaccaggaactgtttaggatgagtgggggcctagagaaggaactctagtcttggggccccagcttccagggcgctaaagggctcaaactggcggcacccggggctacgtcagtaggtcaagccagagcgccggccttcagcagctctgcacccctgacggcctcggttttctctccttctctagagcctctctggacctcaggcccctttgatttggaaaagagaatttcccccacctccactcagcgcatccgtgctcagtccttcctctgggattttcctgagatccccacgcgttggccaaagtcccacgggttttccctttcaatccgctccacatcccagtcccagcctgtgcctagagcctgagttgcttttgcatgcggggcaattgctatctgttgtcagtcctagaactgcgacagcgggtgccagcctgagccttctcaggcagcgcggggttcttaccgggtggtattccagccccctcctgacccctctaatcttgtatgggaaacctgtgctggggagagaaacgcggccttaaaagaaaaggaaaaaaaaaaaaacttaagccgaaaacgcgagagccaccagagagctgttatctgcagctgaaggctgcggtaatcgatgggttatttttacgcggtaatagggccctgtgattgctctattaacctttagacctgtctgagggactctcccctcacagccccgctgcgccagggcccacaggcactgacgccgactccaaactaaggtctgacaccctccctacccccactctagggagggaactacagcctttggatgggttctaacgttggagaccaagctcccaacccccacttcgtcctttgattggaccccagccggtgagggctacagggagaacagggacacgtgtggagggacaggaggccgcaggagttggaagcagtgagggtgaggggtgcgcatttttgtataaatatgtaaactcctgggctggtggcgggttctcggcgcctcgcagtctccaccggctcgtgggaaggagaaaagggcgtttgcgtctctaagcaccgaccttaagggccagttcaggttttagagcctcctgaccctctcccgagctcatttcaagggccctgtagatctgagtccagccaggcaccccgggtgacccagtgccccacaggcgaccatgagcccagagtgccaggtcaccttctcctctaccagccacgccccttgaaaacggcgtctggacttggaccactgagagtggggaggaagagaagaaactcgagaataaacacgatccttggcagagcagcaaaccgcaaggacgtcgggctgcaccgagggcagagcggagcgctggtactgagcactttcattccccgagagatccgcgcgcatggactttgcctgctgggggagctttgggaacagtcccgcgggcgggcaccgggcggaggtgctgccgggtccactctcccccgcccccgcccttgcacgtgactcccttaggccagtcagagcaggcgatcgcaacgcgggggccccgggaaccgctccgggagcaagggacaagccggaggcctgcagagtggtcgcccgaggctccgctgttgctgcagctccgcggactcacggagatcgcgccggctctctgggaaactggagctggccaggactgtcgcttcgaagggaacgggccctcttcgtccttcgctggagccgcactggagccaccaaatgcgcctgggtgtgcagcaggcagaaagcaaggactaggcacctctagcccgtgggtgatccgaaggcctgcgcagggtgttcgagaccagtcttgactgagttctgggcatctccagcctctgggccctggagtagagtcagccctcctgcagtttctggagcaaccacaaatccaatttgggatttttgttttcttcttgagcaaaccagagcctaaagattgttgctctgatgctggatttaattcgtatatattttgagcgagttgggcctgtcctcattgttttttgatctccgttttcgcgacagttctgcacacctgtattccaattcttgtcgctttggtctgccgcgcccgcgtgggctccttgacttgcaaataaaggggagcacaccgaagctcagagcttggggcg

SEQ ID NO: 2 - DNA, 5000 bp, Homo sapiens
cctactgttttctcatcagtgttagcaatcccagggaggttgctggcaaatagaagagttttgatatattcttcattttatttatatcttgagctatacggagattataatatatcgtgcagcatgtgagtgcccaagaagtgccatcctgcatcaagaaatgctctcatcttccatctgttctttcaataaaaactttaaatcattttgacttgagggtcaggcagctgaagatgtgtcacttagaccatcctttcttttctcccccaccctttcctcctgtatttcttttggcacttcctaggcaaatctatctttgaacattttattagctgtacactttttcattcaccacggtgatgcccctgggcatcagtctccagcctgagtcaggagcttacaggagcttctttccacaccttgtctgcttcttatgaggacagcagtccccaggaaaccctttgctaaccagttgcagctactcagaaaaatgtgggtgcttcccacttggaagactggctttgcccagagctgactcccagccttccctcagtggaatctgaaaggttctcagacatctcgggggtctgcctcaggttctgcctgtggcccaaataagagctttagaaatcttaagcaaacaaaaggttttggtactttctcccacattcaattcaaaataaaagccattcttaaccgtgaaatgagtgagaagaagcccaataaagttctaattttcctcctgattatgtcttttatttttaaatgtcaactattaaaatgtctccccaaattgttggcttttttgttgccctacttttgctggtgctctaaccatggtgcccaagtaacactgcccatgagccctgagaggtaacagcaggatgtaaaagagtagtgtctgtggactggtactgtcctatctggaaattgcttttagaggagtaggagaaggtgatcagcattgtgataattgaagcagttttccagaaaagcatagggattcctgggagccagtttcctttccattttttggtcaagaagcaaccagctatttccatgttttctgctccattctgtgtgtgggatagatagatagtgtttctttaacagccccctccccccgcaagagagaagagtctaaaatgtcatctttctggagaatggtggaacgaagaaaggaaattcattttgaaaaggggtctgaagtaagactgttctgagaaagccccttagcaggtagaaagatgcctccttcctacccgcagctcttgcctcttcccagcgcacttcctgtagtaggaaactggctgaaaaagaaaggatgtttgtcacggaggaccctgatgtgggacgttcgtaattcttacccgcaattcttcctggcagaacttcaagccttgagggcaagaacagcaaaggagccgctggagggctgcggcggggactagggaagagagatgcttatagtcttccgtgctcctggagccagaaggagggctgaagtttgctgtagttctagaggaatgcatgcgaactacacgttagaggtggcagcgggggcgagtgctcccacattaaataaaatatggccacataaatgggtctccctgtaagagctttaagctatattgtatttgcacattgtaaagtaaaattcaggtttcttattaggtgtgtgtatacaaacacatgtggatgccaagaaatagagactttagagtttaaatcaactctggtgctgcttcactttcaaggtttctggagactccttctgccccagggtgcaataaatcaagggaaaatgatttttagattgtttcgtatgtgaaacgacccaatggaaactggggaatcttgtgagggttgccagggtggggtgtgaagacagattcctgcagcgttgattgtcgtggcaggtggatcatgatgcacactcctgtctcttcaagcagcagcactgcccccactgtttattcagagcagggaagagaagggcttttatttttttcaagtaaaatttaaggttttcaaacagggtgtgtttgaaacagacagcgccctgagaagtggaggcgctctctgggcctggcgcaggtctttgttccagcgcagcaccccttctccaaaggcactggcaaagtctattccgcacacacagttacactgtcagaaatctgcggatcttagatttgtctccagacaaggctggagaagagagctcactaccagacaaatggcaattggcatgttgccttcctatgagcactttgagatcccacagcccctggctagtgcaaaaggaacctcgccctccactccttgctgacacctgccatttcatcttttctaacattcaagccatagcttgagtttgtggaatggatgtctgtggtggagaagtgaaaaatcggtgctattttttcattatcatgcaaaaattaagacataatcctggataccaaaactcctctcccatcattaaaaaagtaagatatttaagtcgggggtggagggcagagaaagtctacatcctttttgcgagtctctgtgaagagcctgatttctgagaaaaaggttggggaagaacacgcagctgtctttagccagagactgagctatccctggaatgtccagcgtaccctggacctctggatgctctgatgtgtgctggggtgcccagggcactgggttgacctctgtccccacaaacagcttcgaggttgggctcatgacacaaaacgcacagattcacggctttccttcctgacactgcaggcataaaagacagtgttaaccaaagaatcatggactctagaggaattggaagatccttaacgaccacatcctcctagcagaaatttgtattctaaagtgtgagctttggtgcgcgtttcagtgtgcgtgcgcctgcgtgtgtgtgtgtgtgtgtgtgtgtgtatagaatgcatgtcgttaccaaatacctagaacggtattggaccgcaaagaggttaaacccggttgctttcgacaggaatgagttgaaagcacccaagcgttttcgggtgggtatcgccgtccatccattcgatggcctcgctgatgagcatagaagatgaacccgggaggactgtcccttcctggctgcgaaacctgaccgatataggggagattctagaagaaagagctaaggaggccagggcggaaagaggaaagtcaggtgtaggggagattaaaaggtaaactggtgggactgaccgggctggtgggactagttagcgaagccaagtgtttgggtagaaacagagatcttgtaggaggtgctccgagctacatttcgggatttgggatgaaaagagaaagccagggggttcgccgctcctggtttgttccttaccgaccctaaactgtatggcggagcttgtgggtcaggcgtggaggcggcatccactcccacgtgccccacatgcagcagcaatcccacgcggttttcgcaagcggctgggaggcaagcgggcgtccccccacaccccccccccactgcgccctggacccacgccacgcaccgccaaggagtagctatggtggcacgtggaggaggggcgggtaggcagggcggcaccatcaccgggaccagcatcttggaacggtcaatctcactgctcatactctcctccctaccggcgcgggagccttgctgtaatcccctgccaccccatctccgactgtgaggggcgcaccgggtgggggaatgggtaaataactggcccaaggcccagcttccaggtcctccctccactttcccgcggcctgactgcgccccgggcaacacagccccttgggaacctgcgaccaggtaatgtggcctccgccccgtcccgggtctggctgcatcccctctaacccctgcctgcccgggtccaagtgagggttttctcccggaaggttaatcaccacccccatcccctagccgggtcccgggaggaagagatgccccggctctctcgttgacccggcctcgaacgccgacctcgagcgcagaatactcccgagtgctgggttcctgatacccagcccgggagcgaggggagtgaaggagggaccgggacaggaaggcggggacgcggtgggtgaacaatcaatggcttagcctagtttccccgcaggagctaccgcccattcaccgtctatgcaagcaccagctggcggcagcatcttttaggtacaagctgcagggaggcaaaagctggctggggggccgggagcccagggcacgccgcgggactccagtccccctgctgcgccccagcctcaagacgctctgggctttatgaggttcagttaccgccgagtcctccattaacacctcctctgattattgagaggctggaagggctgaacgcaacctcagagcgtttctctgggcaccggcccgcgtctgtgccctctgcccaccccttcctcctccccctcccgatttggaagcataaacagaaaataaaagacaggtggagagagctaagggtcagaggtgagggaaacagatcaactccaacggggtgggggagagagagaagggagaaattaattttcatccaatgctttatttatttatttggattaacagggggaccctcagcgagacgcagggactggcccgcacctctggccgccgcctcccgccgagaaccgagttctagttggggaaacgtctgacatctggacatcaaggtaagaccctgatggagatattttagtcggagcagttttctgccctcggagtgcgcccggacgcccgggagtttcagggacttcttgggagcctaccggaggaaggcccggctttgctccaagccgcgtccctacctgaggtctccacgggaggagcggtttggacgcgccccaccggatgggaggaggtgcttggggaagccgggccgtaccgccggtctgactcccggcttttctctgccagtgcaaccaccattacggcgtgatccactccttttcctctaaga

SEQ ID NO: 3 - DNA, 5000 bp, Homo sapiens
ccactgcactccagcctggatgacagagcaagactctgtcttaaaaacaaaaacaaatgaaacaaaaaacaaacagggtaaatgctatctccctttaataactaattttcagagtatcaaattggtgtaatagacactaccaatgatggaaaaatagtttccctcttctcattctctcttctctgtactagagagtagacttttatttattcagtatttgcaatcaattgtattcattttctttttgacgctaaaattgccctaaatttgccccctgggagatccttcaagctggctcccatgtctctaaaatttatttttgagagactgaaaatacaaatggtaaatggccagaatatacataaaaacagaattctgacccattacctacaacaactagcccagtaaaccaaccacttatttaccataagtgtactctcagcctgaaagccaaacctgctatcaaccagacttgtaggaagtcagactgctatctctggtagcaatccaggaagctaaataataacttctatgacaatcagctcaaaatggccaggacttgattaaaacttgacagcttccctactttttgtcctcacttctagcttatgcccatccagagaaagccaaatatgctcctttaaccaagaccataggatgccaacttgtagttagcttccagcttccccatgccaacagcttccaatcaggcatacctgtgaaacctttccttttgtccactgtaaagctttcccactcctctgcatgcctttgaacctctgctaaaacataagggatggtggctgactcccttgctacagcaagttctcaatcgactttgcttttctcatttggtttgcaataaaatgcattctttacagtgacacaatttgatgaattttgacaaacgtatacattcccactaaccatgacacagaacatttccatcacccccaaaagttctcccattttcctttccagtcaattccttctctcttcccctcctaaccaaccactgatctgctatctaccactatggattagtgttgtttgttctcctaactacagttggcatagcctcattggtctaagcacttcctctgcattcaggcacaacgaaatacctggctcatcttgtacctttcccgtcccagagctggaatcagccagttctccaaaggagctctggatctttttagtgggaaatggaatttagaaaccaagatctcagtgtcagatgtgctcactgttactgtagggtgcccatgcctatacatcctggttcagccaggactgttccagttttaatctgaaagtccagcatccagagaaaacccctctgtttgtggcaaactagaacaactggtcaccctagtgcttctaggccctttcagtccacagaactaggacttttttttgcagttgaaacttttttttctttttgatagggtctgttgcccaagctgtagtgcactggtgagatcacagctcattgtaactttaaactcctgggctcatttgggcctctcgcctcagcctcccaagtagctaggactataggcacaggccaccacccctggctattttattattttgtattttgtagagacaagatctcactgtgttgcccaggctggtcttgaactcctaagccactttggcttcccaaagcactgggattacaggtgtgtgccactgcacccagccctccagtttaaactcaatactacagactttttttttctgaacttcttaaaattctttatattagtttccacccccacccccggatgaatgaacgtcttaaaaattgttttaaattttttaaaaaatggagacagggtcttgctatgttgcccaggctggtctcaaactcctggactctagtgatcctcccgctttggcctatcaaagtgctgggattacaggcatcagccacccggcccttttctgaacttctttgatcttgtgtttgtatctcattctctcacacactgcacatctttgtatatagtaacattaaaatacttatgttatcctaccatatacagtacagagttttaaaaccacagtaacaatgttaccattaatcataaatcagtgaagctgaaagctcctgtgcacttttttcatccttagaatacacagttgacccttgaacaacactggtttggactgccagggtccacctatatgtggatttttttcaccccaacacagatgaaaaatacagtggggcgagggacggtggctcacacctataatcccagcattttgggaggctaggtgggaggatcacttgagcccaggagttcaagaccagcctaggcaacacagctctctactgaaaatagaaaaaaattagctagatgtgctggcacatgcctgtggtcccagctactcgggaggctgagatgggaggattgcttgagcccaggaagtcaaggctgtagtgagccatgatcgtaccactgcactccagcctggacgacagagtcagaccctgttctcaaaaaagaaaaaaaaaaagaaaagcaagagaagggaaggaagggaggggggaagagagggagggagggaagaagggagggaagaagagagggagagaaagaaaaaaagaaaagaaaaagaaaggaaagggaaggaaggaagaaaacaaaacagaagtacagtgggatgcaaaacacacctacacaaagaaccaactttccctatgctcagctccgggactttagtatgcagatttctgtatatgctcgcagaaggtcctaaatccaatctcctgcatataaggaaagactacactatcacaacacagatacactactagagtactgtgttcaaaagctcagaggggccagtggtggtggttcattcctgtaatcccagcactttgggaggccaacctgcaggcggatcacctgaggtcaggagttccagaccagcctggccaacatggtgaaatcccatctctattaaaaatacaaaaatcagcaggggatggtggcgcatgcctgtaatcccagctattcaggaggctgagctgagagaatcgcttgaacccaggagacagaggttgcagtgagctgagattgcaccactgcactccagcctgggcaacacagtgagactgtctcaaaaaaaaaaaaaaaaacccaaagaataattattttttctgtgtatcttagttatattagttaacagttaagttcatttgtttcaatttgctttcaaccttaggatttactttgtttccttttttatttacttttttggctatgtaaaactaaaagttcaaagacagagaaatcttgtctccaccccccactctttataggcaactaaaaatgttaagtttccaggatatccttccaattcctccccgaaaataagcatatacacataaatattcatattaaatataaatatatatttttatgtatttaagtaaatacataaaagcatatatgcttatttttgcttttgaatacagtgaatacttactacacttaaatatttaagtgaatacttaatacaacctgagactcattccaaagcaacacacgattatttcctgaatctttttaatggttgcattgtgttctactgtatggatgcaccattcccaatcagttccctattaacggatatttgggttatccctaacttttgctatgacaaatagcactgcaataaccttgtatacctattgtttccatgtgtggagctgcaccttcagaataaggttagggttagaagtataactgcaaggtataagaataaacacaagtaattttgtactccgtggaacttgaaccatttttccactcccaccaggaaaggtaatttcaacgtgtatttccattgtaagtgaggtggagcgactttctatgtttaaagaccattacggtattttttctgagaatcatctgttcatgtccttccccattttctcctttggcttttgcactttttgttttcaatttttgagagctcttgtgatacgagttgcaaagattttctccagaccgatctgattcactggaaatatactacgaccatctggaatgtactttaatcttatcctaataaatcaccttactacaagacactcctttggggatgaacaggtttgataaagccggctaaggacagaccaatcaaaagaattacgtgctcttctagtaagtgtttagagactgtttcacgttctccaggtaacaccgccattactcggcggtccagatggcagtcttacttcagggagtctactccacttgcaggacctctttgattgagttcactaaacctcacagggccgccttccccgtgttccagttagcaaggggccctcaacgccgcggacacagcgcaacctccgacgccagagaacaatagctcctcgatgcgtctccagagatgtcaaggaggaacgaacccagcggccaggagactgcgcctcacgactgatgagagggaaggcatcggtttctaagttctcaaaacttacaggtgagcttctgctaagagtaaacgcccgcggctcgccgcccacggcctctctcttgaggtggcacctggtcctccgacacgcggaagcatcacggatgagcgtcacgaacacagagcggccaatcacgcgccgccttcgcccagatccctccgcacgaggcagccccgcagccgcaggtggccccggcgagtacttccaccttcccttcccgtgatgccctggggctgacctccgacctcgctggcccgcccctcccctcggccgcttcccttactgagcttgctgagctccggggcccgcggagctcgcgccaggctcctgggaaaggacggggagtgttaccggggagcagctgctccattgtgcctcgaggccccgatcgggctaggccgacggcctccctcccttcacctttcctctcctggcggggttcggcggcgggcgagtgactgcggccacgcctgaaaggcgactctcctgattcaag

SEQ ID NO: 4 - DNA, 5478 bp, Sus sp.
cctgtaagacctccttatctcaaatgcatttatatgtatatgtacagagtttcaaatgcacaaatttgatttagttgggcaatatatcacttcacaaagtttcatgcttctgacttgactaaattacaatctgactgtaataactcacatttatgagatattgttaatcatttcatacacactttgtgaagtaaacacagataatatatttactctcattttcaagattatcaagctagaaactgcagtggtatttggcagaagggacctagggtctttccaccagggctgccgaggttcttcaaatccactcagtcacaggaaacgagaaaaggctgggcaacgtagtgaagttaaaaacatgtgtagatgtggaagaaacttcactctgtttcagtaagcgggtgtttactgagtgccttgaaaatgccagggaatgtacaaatgactttaggtgtgatggttgtggggagtgagtcatgaggaggaggttaaggagaaaagaaagcaatttcctaggagacagatattaccacagttccagacaccaatacagggggagtctgtaactaaaattatcaccaccattaggggacagagcaatatggaccagcagagcaacaattttcaaacttaccagtcaaatcaattttaatagcttgttaaacaattgggcaatcatttttaaaccctgtcggggcaactaaattaataaattagataattcgatcaacatctggtattgggacatattcaaaatcggtagatgcagcaacataaattgatttgacggacgtttttcccttgagcagactggttgtttgactgacttagaaagctgacttgagaacatttcagaattactgtgaatataagtcattattccagctcagcagctatgtgacccctaaggcaatggattaccttctttgtagctcatctgaaggaaataaataatgataacttgatcttctgtaggagtttcatgaatcagcagctaaagcctattaattcttccataaatagagcttccaacattctgggtcttggaaagaaagaggagggaggctatttcttggattttatcaggcactatttttctacaatagtgtttacccaatacctacattgaaaagtaggaaaacactattgttactttctgtcacctgtttactgaaggaaccaaattctgaggcattccagctcttttaagcctggattgaaaattcacagtcaggtttcagatcgccatgcctttccaggtcaacagcagcttgctgtgcaaaggtcagttttacaattttacccggctatatccccacctgtacttcactcctccatcctttaatgaaaactgcattactttgaagttgaaggttgcattcaggcaggaaaagcagctttactcccaccctgagttggaacagataatggattctgcagagcaggtgtccaattgaccaaggtcaccgaacaatgactcagtgtaaatatccaaattgcagttgtggacaagctcagcacataacaggtgcagttaatgagtgtctttttgtctagctgtcacagtggcctgaggacatggaacaaatcaaacgaagaaggctgtgctgacttagaaggttatgtgtttactaatcctatggcacggagcaggctggcagaacttgatgcaaacctctgcacttgtttggacaattttgttactcaggagctatttgttcagggggaagaaatggaagtggaaagatgaagttagttttctcctgacatttccaaggatgaatacttgtgaaggacaaacaggcaaactctccttagaaagagtcaatccaaagaacctagaaagtccttcagagttttgaatacaagacagtaacttcaagaaaagcacaagtaagaaaatcagaagactgctcttggaaaagaccaggaattacaaccaatgtattataagttcatggttctcagcactgcactgaagtttcaatctgggacaatgtctgaaagtcaaagtcaaaggcatcaaatatgtgtgagcatatgaagattaaagtcacaaaaaaattggtatagatgattaaagtcacaatcctaattggttattaggattaaatatatatatagcttataagatgagttttagaaaagcccttaaattgtgcatagctaggatacgtatattaaaacagtttatagcattttgttctagttgcagagtggaaaaggctagcataatgttaaattcttcgctcagtgttaaatcattagacagtgaatgaggggaaagttcattgcgaatttgaatggttaagttcctttatattttcaacttagtgaagaggtcagcaaacatttctgtaaagagctatactgagagagaaataagaagaacccttgaaggacttagaacatgctgatcacctgtgatctgtagaactgttccctatttcacaagtgtgaaaactgaacttaaatgacttgttcaatgttatacctttagagtggaagggcccaaactcaaagccatgtcttttgactgttaattcaagttctcttgtaactttaccagcagcacctgtagcacacacttagaacaggctgatcacctgtggtctatagaacttttcctatttcgcaagtgtgaaagctgaacttaaatgacttgttcaatgttatacctttagagtggaagggcccaaactcgaagccatgtcttttgactattaattcaagttctcttgtaactttaccacagcacctgtagcaccctgcataaataaataaataaatacctcaaaaaggctcctagtctaacagggcttcctaacttctcttccctccaggctcagtgaagccacagtccctctgagttgaaggcaggagttacggtcttctcctccttctctaaagatatagttggccaacaaatgcattttcaaacacttactttctgccagatgttattctaatcactgggatacagcgtggatgattcctttccctcaggagacttatactagttgggggatacaatcaacaagcattaaataactaaatacaccaatcacatcagattacgataagcattagaaaaaaataaaatggagtaacagaataaaaagcaactagcaccggcaacctaaagaggggagtaagataggtggtgcttttcaggtaggttggttgatgggttcatccaacaggagtaacaatacccaaaacagtagcttgaatattggaaatggcaaatgatgacaaggtaaatgagataacatttccaatatatattattgcgatggaccaggtgacccttccagtgttttgaatctgggccatgaaatgtggacatgcagaaaaaaatgagacggcttcagggtggagggtgagggtgggcttccggaggagagagggaataatggtgttgctggggtagtactacctaactctgaagtgatcccacattatctttaaaagggggaggttaatggctgatacatccaacacttatcatgtaccaagcaaacttttacatgaattaactcaacacaggaggatcacatcacagagactattgtccatgttctacaaaaaaggaaactgaggcacaggtggcttgagtaactgtcttgtggaaggttaataatgcctctgatgagggccatgtcctaattcgcagtacctgtgagtggtagcctacaggtaaaaagacttcccaaatgtgatttaattaccatctttgagatggagaggttattctgtattatctgggtttgcccaatatagttataaagatccttataagagacaaaaagaagatggaagcagaggtaggagtgatgtggccaagagctaaggaatgcaagcagcttctagaagctgaaaaggcaagaaaagagattctcctgtagggccttctacagaaggaatgtcaccttgactttagccccagtgagactgatctcaaacttctggcccccagaactgtaacataataaatttgtgtttccttaagccaactaaatttgtggtaatttgttacagcagcaataggaaactaatacattgcccaaggccctggggctatagagtatcagggccagaattaaaatcaaagcaatctggtgccagcatggagatttagacactgtgtaagacagcctttcgaggcagaataaaatttcaggttgaatccaaaataagtgtctctgagcttttccacttatgcttagaattccaggtctcagagaaacttagcgagaaagagggagggagagaaagaggaagccggggtgggggggggggaggcagaaggaaatagctgggattctataccagctggtatataccagtccccttccctgttccatgacttcagtcatgacccaagagataacgaaataaacttcccatttgagagatgaggaatttgaggactaaaggggttgtggtttcaagttgatcagatggttggtgaaacatcagctagttacacaaggggcaggtgatgatgtgggaattcccagttatcttagtgcctgcaaggaactcaaggaaagagggctgggtggcaagcgaggactaaaggatccaggctttctgaaacctgtttctcatgatttgcctaggaagagtgggagaagaaatattaattgcacgtctacccaatgtaatatgtgtacaagaatccaaagttctcactgtagcaaccttttagccccttgttatgattatattcccaaatattagccattttataagctggatgctgcctctagctctctaacacgacacctacgacagaatgagtgctcataaatattgcttgaattaaacaaagagcttatgatagcatctttagaccagtgatgtttcaaaatttaagacgcaaacaaatcctttggatacttcgttaaaatgagattcagagtaagaaggtctggttggggcctgggattgcgcatttctatcaagttcccataggatgctgatactgctggtccacaatctatatttttcaaggtctctgaaaatgagacctccctatccacatacaaaataaaagtcgtacaaagtttaatcatttaattaaagttcgcaaaatagcatagaaatagatttttcctcttttcggtgccactatcctcccagtaaagggagcaaattagtgccagggtttagtgaaaagagttggaacagtgcccccaaataccttctccctgcgctctgccttcttcctctcattgcactcttccctcctccaggaactcccacgcctccgctcctcttccattttcagcctttaagaagcctctgccccttttgaaccgccgaggtcacaagcgaacacgcccgagcgccgcgcccctctccgcgcctttgctcaggcggagacggcagacgcgcgtgcgtgcgcgctcaatataaatggatcgcaggcggccgctactggacagagccgggctacccgcgcagccc

SEQ ID NO: 5 - DNA, 2605 bp, Sus sp.
gagaggctctatttttcattttctttttctgatatcattaaacatacataataataatattcattccattcattttaaagatctaaagtgtacaatttggtagctcaagagcattcacaatgttgtgcacccatcatctgtgtctagttccagaatgttcatcaccccaggaagacaccctgcacctcttggcaattaccccccctgccctccttccctcttattcagtcctcggcaaccatcacagatctggtccctgactcttatgtatttgcttcttctgtaaatgaaatcattcagtatatggcctctggtgtctgcccttttttttcacataaggtgatgttttcacataaggtgatgatgttttcacagtgcttccatagtccagaatgtgtcacttctttgccttttaatttcaaatttgaaatgatatatatttactttttttctcatatattaatttctagctttacaaaatctgcttttatgtatgagcgcaatttaaacacttgagtctgtaccaatacagctgctttaaattagatcaagtaacagtatttcatattttatttgagtctgaacactaaacttctttgggtgactaagatacaattatggaatttatacatgctttttgaatgaataaagttaatttttgtttggactttttaatattattgtaccataacacttattgcttgaacatcatttggtgatgcagattatttgtatgtgatgcatttaaattttattttctcatgcattgtggcatcattcttatgcagtcttggtgagacttatttttacttttcataggttatgttctgtgcaatctacagtcattactcccatttcccaaacatggttttaaaactgtacggggacattcctcctattttagcacaatgtaagtattttatgacaacttccaagtctgccttaacaatctatttggaggaagtattcacttatttcatttggtaagccatgacctttaaattttaaaaatgttgtcctaagattgaactagaataaaaaaaatgatgaatcagtggctgtaatttaaaaaattagattttgtaaaagcattgttcttttcattttgttctgttttgtacattagtgatgtaaaagtgaagaagacagaggccagtggtgagcatactcctgacagtctcgagaacaaaatggtccctttgaagtgttatgttcacctcgggaaactttgagttccaaggttatctgtttttctcttaatggaaccattttaagtctcttgccagaattcagcctctgtctgccagtgccgaattgaaagaattgaaacgcagagatggggtttggggtaaagaaaaaaaaaatagctttattgctttgccaggcaaaggaggatcagagcaggctaattaatgccctaaagactgtgaagactgtgctcccccacgccccaccccccccccaaagaattgtgaggagtttacagtaaaaaaggagaaaaacaggtttgccgatagaaccggattgggacacacatgcattcttctttctttgggggaatctttgtcctcaaaactgagtcagagatgttcatgatggtggtcttctgggttattgcctaggataacagtgcttgcaaaaagggcgtactgatcagagattagaacaaaccaggaaagttcctgacaaacatcaggtactagtcatctttaacccacaggccattatgctcagagggcctgacctttagcttagaggtggtcctgtttagggtgcaattaagtcagggagaaggacaaggaaggactctaagctgtttaacttcaaagtagtcaatttctaaaagacacataaggaatataacttttctctcaggagtatgaggcaacctgctacatatctaatgttatttgactctgagactggccattaaatagatgctagtatatattaacatactgataatcatcagcagaggtattcacatttagaattcccaattatttgcccacaggaaaacttaacagaattttcaatagttctttaagtagaagtgaagaaagaagaatgacgactagcattcaactctataacatccgccgcctggcaggccacgtgcctgcattcttgcctgtgggcctgagtgctctaatggccgaaaggaaaggaattgggttatcttgaatctctgtgttaaacatagcatttacatatgctcataacgataaaccattgtcagatgctgctgcaccgtcatccttttaattgagtggataaaataaaataacttaataatgacaaagtttccggttttttaaggttagcagagccttcagcaacttcggattagacctagaacagaaagcaagccttattatcataataagtaagcagttttgcttgagaatgggtaggttggttcggctttggctggctggctgcctgccgaccccagcggtttccaggggaggtactgggggcggagaaattggtatttcactacaaagattagagttactcaaatctctggtggaaataacctttaaatagtgaagacaactttccaaacgttacgcttttgatttcgcttcctcccccttttcaa

(19) United States
(12) Patent Application Publication
(10) Pub. No.: US 2006/0236415 A1
(43) Pub. Date: Oct. 19, 2006

(54) Neural crest cells specific promoters; isolated neural crest cells; and methods of isolating and of using same

(76) Inventors: David William Silversides, Ste-Madeleine (CA); Nicolas Pilon, St-Valerien (CA); Diana Lynn Raiwet, Ste-Madeleine (CA); Robert Viger, Saint-Romuald (CA); Eric Legault, Sherbrooke (CA)

Correspondence Address: Schwegman, Lundberg, Woessner & Kluth, P.A., P.O. Box 2938, Minneapolis, MN, 55402, US

(21) Appl. No.: 11/371,402

(22) Filed: Mar. 9, 2006

Related U.S. Application Data
(60) Provisional application No. 60/659,398, filed on Mar. 9, 2005.

(51) Int. Cl. A01K 67/027; C12Q 1/68

(52) U.S. Cl. 800/14; 435/325; 435/320.1; 435/6

(57) ABSTRACT

A vector comprising a promoter sequence driving the coding sequence of a visual marker protein, wherein the promoter sequence functions specifically in neural crest cells and methods for using same.

### CROSS REFERENCE TO RELATED APPLICATIONS

This application claims priority on U.S. provisional application No. 60/659,398, filed on Mar. 9, 2005. All documents above are herein incorporated by reference.

### FIELD OF THE INVENTION

The present invention relates to promoters specific to neural crest cells, isolated neural crest cells, and methods of obtaining and of using same. More specifically, the present invention is concerned with attaching to visual markers promoters that are expressed specifically in neural crest cells including neural crest stem cells, producing transgenic animals expressing these markers, isolating these marked cells and assaying these cells.

### BACKGROUND OF THE INVENTION

Neural crest cells are a population of cells identified transiently in the early vertebrate embryo that are of importance both developmentally and clinically because of their particular origin, behavior, and developmental capacity. Neural crest cells derive from an ectodermal origin. Presumptive neural crest cells are first identified as epithelial cells on the lateral margins of the neural plate, where they are induced by interactions between cells of the neural plate medially and cells of the surface ectoderm laterally. With folding of the neural tube, these cells are placed on the dorsal margins of the neural tube. It is from this position that neural crest cells undergo first an epithelial to mesenchymal cell phenotype transformation, followed by an extensive cell migration (with concomitant cell division) to occupy diverse regions of the developing embryo, and to contribute to diverse tissues and structures in the adult animal.

Migration and Differentiation of Neural Crest Cells Throughout Embryonic Development

Initially, two regions of neural crest cells are identified, one in the cranial region of the embryo and the other in the caudal or trunk region of the embryo. The troncal neural crest cells form after neural tube closure, about three somites rostral to the most recently formed somite. Emergence and initiation of migration of troncal neural crest cells thus follows a rostral to caudal wave; migration is in a dorsal-ventral direction and lasts about 9-12 hours. Two troncal migration pathways are described, a ventromedial pathway and a dorsal pathway. In the ventromedial pathway, the neural crest cells migrate in a segmented fashion through the rostral portion of the sclerotome of the underlying somite but are inhibited from passing through the caudal portion of the somite; it is this segmentation that gives rise to the segmentated pattern of peripheral nerves within the adult body. Ventromedial migration consists of an early phase (beginning e8.5-e9.5 in the mouse) wherein cells will migrate to more ventral destinations such as the sympathetic ganglia, dorsal aorta and adrenal medulla, as well as a later phase (e9.5-10.5), wherein cells give rise to dorsal root ganglia and Schwann cells. In the sacral area of the embryo, neural crest cells of the ventromedial pathway will contribute to the caudal portion of the enteric nervous system (see FIG. 1). The dorsal pathway of migration of neural crest cells occurs about 24 hours later than the ventromedial pathway. This pathway is not segmented, and occurs between the dermatomyotome of the adjacent somite and the epidermis. The dorsal pathway provides neural crest cells that will give rise to melanocytes. The troncal neural crest can be further subdivided into a vagal region that will contribute the majority of cells to the enteric neural network, as well as contribute cells to structures of the heart.

In the cranial region of the embryo, neural crest cells contribute to neural as well as connective tissue structures of the head. In this region, emergence and migration of cranial neural crest cells occur even before neural tube closure, and this time in a caudal to rostral direction. In the midbrain region, this occurs around the 5 somite stage, while in the forebrain region this occurs around the 10 somite stage. Migration is dorso-lateral and segmented, this time with cells traversing the rhombomeres. Cranial neural crest cells make major contributions to the branchial arches, with migration towards the first branchial arch beginning by about e8.0. Neural crest cells in the head region, sometimes referred to as mesectodermal cells, can function like mesoderm and form bone, cartilage, muscles and connective tissue in the head (see Table 1). Other derivatives of cranial neural crest cells include the odontoblasts of teeth, sensory cells of the inner ear, pigmented cells of the iris. A subpopulation of cranial pathway neural crest cells, in the cardiac region (which overlaps somewhat with the vagal region of the trunk) provides cells that contribute to aortic arches and the cardiac outflow tract, endothelial cushions of the heart, and the septum between the aorta and the pulmonary artery.

Migrating neural crest cells follow tracts layered down by extracellular matrix proteins including fibronectin, lanins, vitronectin and collagens, which provide permissive pathways. In addition, some tissues, such as the caudal portion of somites, may contain inhibitory signals that block or disallow neural crest cell migration. Recent studies suggest that migration of neural crest cells is a communal affair, involving synchronized streams of cells in communication with each other rather than individual cells. Thus cell-cell interactions remain important, and migratory neural crest cells express Cadherin 7 proteins on their surface.

As well as migration, neural crest cells undergo cell proliferation, survival and differentiation during normal development. Classical transplantation studies have revealed that neural crest cells undergo progressive determination by responding to the regional environment they find themselves in to coordinate cell lineage development and generate tissue appropriate structures. Tissue patterning and cell commitment of neural crest cells is a balance between permissive and instructive signals, and is influenced by cell-cell contact and by soluble environmental factors. Cranial neural crest cells are distinct in their fate from troncal neural crest cells, but within the cranial region remain pluripotent. Recently it was shown that FGF8, secreted in the craniofacial region by cells of the “isthmus organizer”, will modify HOX gene expression within neural crest cells, and thus affect branchial arch patterning. Cardiac neural crest cells may be the most predetermined (i.e. the least pluripotential) of the neural crest cell populations. Troncal neural crest cells retain a high degree of developmental plasticity, responding to cues found within the local environment.

Neural Crest Stem Cells

Stem cells are undifferentiated cells that are characterized by their ability for self renewal on the one hand and on the other hand their capacity to differentiate into diverse cell types. This is accomplished by asymmetrical mitosis wherein cell division of a stem cell results in one daughter cell that remains a stem cell and the other daughter cell that has initiated differentiation to a mature (but restrained) cell phenotype. Examples of stem cells include embryonic (ES) stem cells which are pluripotential, and in adults, hemopoietic stem cells which can give rise to the different blood cell types, primordial germ cells, and nerve stem cells. Neural crest stem cells are also described as a renewing population of undifferentiated neural crest cells that retains the developmental capacity of differentiated neural crest tissues, i.e. can produce nerve cells, glial cells, neuroendocrine cells, as well as muscle, cartilage, bone and connective tissue cells. Classically, neural crest stem cells are described in the embryo; more recently, neural crest stem cells are postulated in two adult tissues, the skin and the gut (Kruger 2002; Sieber-Blum 2004a,b; Fernandez 2004).

Stem cells find applications in many medical fields but research is hampered by the limited known sources for these cells.

There remains a need for additional sources of stem cells and particularly of adult stem cells.

Genes, Diseases and Syndromes Involving Neural Crest Cells

Neural crest cells undergo complex developmental processes involving cell commitment, migration, division, survival and differentiation. Understandably, many genes are involved, coding for extracellular matrix proteins and signaling factors, membrane receptor and cell adhesion molecules, intracellular signal transduction molecules, and nuclear transcription factors. Numerous genes are now associated with developmental lesions involving the neural crest cells and their tissue derivatives. A number of mouse models involving congenital anomalies of neural crest derived structures have been identified and engineered, and several medical syndromes are described in humans. In addition, environmental factors may also have a role to play in the etiology of congenital anomalies involving structures derived from the neural crest.

The endothelins are a family of bioactive peptides that have been associated genetically with neural crest cells. Endothelin 1 (EDN1) is a potent vasoconstrictor peptide produced by vascular endothelial cells; Endothelin receptor type A (EDNRA) is one of two membrane receptors for the endothelins. Developmentally, EDN1 and EDNRA are important for postmigratory neural crest cell development, including the formation of craniofacial structures, pharyngeal arch arteries, aortic arch structures, and the endocardial cushions. EDN1 is also expressed in the dorsal ganglia and spinal cord, and is highly expressed in the adult lung. Mice that are null allele for EDN1 die at birth due to respiratory failure, and display developmental anomalies of pharyngeal arch derived structures including cranio-facial anomalies and cardiovascular lesions. It is of significance that at least in cardiac tissues, GATA4 will transactivate the EDN1 gene promoter. Endothelin 3 (EDN3) is a growth and differentiation factor for neural crest cells, and is involved in glial-melanocyte cell fate decisions. Defects in endothelin 3 and/or its receptor, Endothelin receptor type B (EDNRB) will cause disrupted migration of neural crest cells of the melanocyte and enteric neuroblast lineages, resulting in aganglionic megacolon and pigment disorders. Defects in these genes can be the cause of Hirschsprung disease in humans. The Lethal spotting (ls locus) mutation in the mouse involves mutations in the endothelin 3 gene. The Piebald lethal mutation (S locus) involves mutations in the EDNRB gene, and features megacolon, spotted coat color, and homozygote lethality. Endothelin converting enzyme 1 (ECE1) encodes for a converting enzyme for the endothelins. ECE1 knockout mice reproduce the phenotypes of Edn1 and EdnrA mice (defects in cranio-facial and cardiac structures), as well as the phenotypes of Edn3 and EdnrB knockout mice (lack of enteric neurons and melanocytes).

The RET gene codes for a membrane receptor tyrosine kinase that acts as a growth and differentiation factor and also as a potential oncogene. Mutations of the RET gene are associated with Hirschsprung disease as well as with thyroid carcinoma and multiple endocrine neoplasia.

Fibroblastic growth factor 8 (FGF8) is a secreted epithelial factor involved in the processes of gastrulation and organogenesis. FGF8 is involved in banchial arch patterning, and is secreted by the “isthmus organizer” in the craniofacial region, causing the expression of HOXA2 gene in cranial neural crest cells. FGF8 also functions as a left determinant (in opposition to SHH action). Fgf8 as well as Fgfr1 floxed mice are available.

The Patch gene (Ph locus) codes for the receptor for Platelet derived growth factor, and is expressed in mesenchymal derivatives of cranial neural crest cells. Mice that are homozygous mutant or null allele for this gene have lesions suggestive of a defect in cardiac neural crest structures, including cranio-facial, septal and ouflow tract, and thymic lesions.

Sonic Hedge Hog (SHH) is a secreted signaling protein involved in the developmental establishment and maintenance of early midline structures including the notochord, neural floorplate, and neural tube. SHH is also required for cardiac morphogenesis and proper cardiac looping, and is a right determinant (in opposition to FGF8 action). Fetal alcohol syndrome blocks SHH signaling in pre-migratory and migratory cranial neural crest cells, resulting in the death of these cells and the characteristic facial anomalies seen with this syndrome (Ahlgren 2002). The Patched gene (Ptc locus) encodes the membrane receptor for SHH.

Nerve growth factor receptor tyrosine kinase (NTRK1) is a membrane receptor. Mutations in the NTRK1 gene cause peripheral nerve lesions including congenital insensitivity to pain (resulting in self mutilation), anhidrosis, and abnormal temperature control.

The peripheral nervous system (sensory and autonomic neurons, but not sensory neurons) is a product of neural crest cells. Neurotropin 3 is a secreted factor that regulates the survival and differentiation of developing peripheral neurons. Knockout of the neurotropin 3 gene in the mouse results in loss of peripheral sensory and sympathetic neurons as well as cardiac defects.

SOX10 is a DNA binding protein and transcription factor of the SOX (SRY related HMG-box) family. SOX10 is expressed in enteric ganglia, Schwann cells and melanocytes; mutations can give rise to aganglionic megacolon (Hirschsprung disease). The mutant mouse line Dominant Megacolon (Dom locus) is characterized by heterozygote megacolon and dominant white spotting and homozygote embryo lethality. The genetic lesion is a premature stop codon within the Sox10 gene, with consequent loss of neural crest cells due to apoptosis.

PAX3 is a nuclear transcription and developmental factor of the PAX family. Mutations can lead to one form of Waardenburg syndrome with congenital anomalies including hearing problems, craniofacial and limb anomalies, aganglionic megacolon, pigmentation deficits, and neoplasia. In mice, mutations in Pax3 give rise to Splotch mice (Machado 2001). PAX3 and SOX10 synergize to activate the MITF gene, whose gene product is necessary for melanocyte development and survival; failure of melanocytes to survive results in pigmentation and also auditory defects. The mouse Splotch mutation involves a mutated Pax3 gene; Pax3-Cre mice are available (Li 2000).

Heart and neural crest derivatives-expressed 2 (HAND2) is a basic helix-loop-helix transcription factor expressed in the heart and needed for proper formation of the right ventricle and aortic arch arteries. Significantly, HAND2 interacts physically with the C-terminal zinc finger domain of GATA4, and together they synergistically activate cardiac specific target genes. Mice that are null allele for Hand2 die at e9.5 due to major defects in vascular development. It is not clear what the full role of Hand2 is for neural crest cell migration and differentiation. Hand2-Cre mice are available (Ruest 2003).

GATA3 is a GATA family member that is most noted for its differential expression within cells of the hematopoietic system, being expressed by T cells but not by B cells. Knockout mice die in utero at about e11.5, and show severe deformities of brain and spinal cord, general growth retardation, problems with hematopoiesis, and internal bleeding. Pharmacological rescue of knockout embryos reveals further defects in structures derived from cephalic neural crest cells. Mutations of GATA3 in humans are also responsible for human hypoparathyroidism, sensorial deafness, renal anomaly (HDR) syndrome.

Mammalian achaete-scute homolog-1 (MASH1) is a basic helix-loop-helix transcription factor involved in mammalian CNS and neural crest development. Mice that are null allele for Mash1 die at birth from breathing and feeding problems, and in addition have severe structural anomalies of the olfactory epithelium and the sympathetic, parasympathetic and enteric ganglia. MASH1 is a mammalian homologue of the achaete-scute family of developmental factors seen in Drosophila, which are involved in neuro- and sensory development. Significantly for this discussion, achaete-scute proteins can associate directly with Drosophila GATA protein homologues; to date such an association has not been described in mammals.

Micropthalmia-associated transcription factor (MITF) is a basic helix-loop-helix zipper nuclear protein. Mutations in MITF result in pigmentation lesions, eye anomalies and deafness, and can be responsible for some forms of Waardenburg syndrome involving aganglionic megacolon. The MITF promoter is activated by PAX3 and SOX10 proteins and the MITF protein is required for melanocyte differentiation. Furthermore the MITF protein functions to activate the tyrosinase gene in melanocytes.

Several additional mouse mutants show aganglionic megacolon and/or white coat spotting (‘piebald’) phenotypes, indicative of neural crest cell involvement. The Steel (Sl) mutant involves a mutation of the Kit ligand gene (Mast Cell Growth Factor). The Dominant White Spotting (W) mutant involves the c-Kit gene which encodes a transmembrane tyrosine kinase protein that is the receptor for Kit ligand. C-Kit is expressed in structures within the brain, melanoblasts, germ cells and in cells of the hematopoietic system. C-Kit promoter-Cre mice are available (Ericksson 2000).

Diseases and Syndromes Involving Neural Crest Cells

A number of diseases and syndromes involving neural crest cells and their derivatives are described in humans.

The defining feature of Hirschsprung disease is aganglionic megacolon, which occurs more commonly over a short segment and less commonly over a long segment of the bowl. Genetic lesions associated with aganglionic megacolon can include mutations in genes for RET, EDNRB and/or EDN3, SOX10, GDNF, HOX11.

Waardenberg syndrome combines anglionic megacolon with deafness and pigmentation defects. Genes implicated include MITF, PAX3, as well as EDNRB, EDN3 and SOX10.

DiGeorge syndrome involves the disruption of normal cervical neural crest cell migration into pharyngeal arches and pouches, resulting in craniofacial and palate defects, and outflow defects to the heart. Genes potentially implicated include TBX1, CRKL, TUPLE1, all located within human chromosomal region 22q11. The VEG1 gene product, by physically interacting with the TBX1 gene product, may result in similar developmental lesions when mutated.

CHAR syndrome involves facial deformities as well as heart and limb deformities. The gene involved is TFAP2B, a transcription factor found in neural crest cells.

Several malignancies are associated with neural crest cells and their tissue derivatives. Neurofibromatosis is a common genetic disease featuring tumors of Schwann cells and nerve sheaths within peripheral nerves. The genetic lesion is within the NF1 gene, which codes for the neurofibromin protein; this is a multidomain protein that can regulate several cellular processes and can function as a tumor suppressor protein. The NF1 gene codes for the neurofibromin protein, which is contained in non-myelinating Schwann cells, and not in myelinating Schwann cells. The NF1 gene is large, spanning 350 Kb and involving 60 exons, and is noted for its very high mutation rate. Tumors tend to have a phenotype consistent with that of an immortalized pluripotential neural crest stem cell. Other malignancies involving neural crest derived cells include melanoma, an aggressive tumor of melanocyte origin; neuroblastomas, a childhood cancer found in the autonomic nervous system; pheochromocytoma, a neoplasm of the adrenal medulla; and multiple endocrine neoplasia.

There is therefore a need for a tool to identify neural crest cells, for their purification and for their availability and use in in vitro and in vivo applications.

A need also exists for neural crest cells, capable of differentiating into neural crest cells tissue derivatives as described in Table 1 for instance and that are capable of proliferation in vitro and thus amenable to genetic characterization and modification techniques.

It is another object of the present invention to provide a method for the in vitro proliferation of neural crest stem cells, to produce precursor cells available for transplantation that are capable of differentiating into neural crest cells tissue derivatives as described in Table 1 for instance.

There is also a need for a tool for a more systematic genetic profiling of neural crest cells from different tissues and at different times of embryonic development. Current gene expression profiling of neural crest cells is preliminary (Bronner-Fraser 2002; Gammill and Bronner-Fraser 2002; Buchstaller 2004) and a full catalogue relating regionalizing signals for neural crest cell lineage development over time awaits the future. There is therefore a need for tools enabling observation and characterization of neural crest cell lineage development over time.

Pancreatic development

Pancreatic development initiates with the invagination of the endoderm in the area of the forgut-midgut junction, to form the ventral pancreatic bud; this occurs at e8.5 in the mouse (Murtagh 2003; Wilson 2003). One day later, a similar dorsal pancreatic bud is formed. Cells within the ventral and dorsal pancreatic buds are non committed progenitor cells that can give rise to all three predominant cell types found in the adult pancreas, including the cells of the pancreatic ducts, the exocrine acini, and the endocrine islets. These three main cell lineages have separated and committed themselves by e11. The endocrine islet cells further differentiate into A, B, D and pp cells; it is the B cells that secrete insulin.

Classically, cells of the neural crest are not felt to be involved in the formation of pancreatic tissues. However, Gata4 expression has been noted in pancreas development (Wilson 2003).

There is also a need for a tool to identify pancreatic cells, for their purification and for their availability and use in in vitro and in vivo applications.

A need also exists for pancreatic cells, capable of differentiating into all three predominant cell types found in the adult pancreas, including the cells of the pancreatic ducts, the exocrine acini, and the endocrine islets including the cell types A, B, D and pp cells, and that are capable of proliferation in vitro and thus amenable to genetic characterization and modification techniques.

It is another object of the present invention to provide a method for the in vitro proliferation of pancreatic cells, to produce precursor cells available for transplantation that are capable of differentiating into cells of the pancreatic ducts, the exocrine acini, and the endocrine islets, including for the latter, A, B, D and pp cells.

There is also a need for a tool for a more systematic genetic profiling of pancreatic cells at different times of embryonic development. A full catalogue relating regionalizing signals for pancreatic lineage development over time awaits the future. There is therefore a need for tools enabling observation and characterization of pancreatic lineage development over time.

The present invention seeks to meet these and other needs.

### SUMMARY OF THE INVENTION

The present invention is concerned with the identification of 5 promoters that function specifically in neural crest cells, namely promoters to GATA4, LHX9, WT-1, DAX1 and SRY.

It is further concerned in a more specific embodiment with the identification of promoters that also function specifically in pancreatic cells, namely promoters to GATA4, DAX1 and SRY.

The Transcription Factor GATA4 and its Family

The GATA family of transcription factors consists of zinc finger proteins that recognize and bind to a trademark GATA DNA motif (WGATAR) found in the promoter regions of numerous target genes. These factors are involved in numerous biological processes including developmental decisions, cell proliferation and migration, and regulation of tissue specific gene expression in a variety of tissues, notably those of mesodermal and endodermal origin (Patient 2002). The GATA family is not large, comprising of only 6 members in vertebrates. Two sub families are recognized: GATAs 1, 2, and 3, which are expressed within the haematopoetic system, and GATAs 4, 5, and 6, which are expressed within endodermal and mesodermal tissues, notably within smooth muscle and the heart (Molkentin 2000).

GATAs 1, 2, and 3 are expressed principally within blood lineages, and function as master developmental regulators of erythroid and lymphoid cell identity. GATA1 is expressed within erythroid and some myeloid cell lines. GATA2 is involved in the activation of genes essential for the development of all blood cell populations. GATA3 expression is necessary for defining T-helper cell populations. Interestingly, haploinsufficiency of GATA3 results in hypoparathyroidism, deafness and renal anomalies, suggesting a wider expression profile than just the haemopoetic system. Mice that are null allele for Gata3 have revealed a role of this gene in the sympathetic nervous system and cranial neural crest structures (Lim 2000).

GATAs 4, 5, and 6 are broadly expressed in mesoderm and endoderm derived tissues where they regulate cell type specification and contribute to tissue specific gene expression by interacting with other tissue restricted and semi-restricted transcription factors. In particular, these GATA factors have been associated with smooth muscle and heart development. Current literature describes GATA4 expression in the embryo within the visceral and parietal endoderm, the heart, gonads, intestines and liver, while in the adult GATA4 is expressed in the heart, lungs, small intestine, liver and gonads. Knockouts of GATA4 embryo are lethal in the mouse between e8-e9, due to lesions in ventral closure of the foregut and to defects in cardiac development. Within the heart, GATA4 transactivates several cardiac specific genes, and is up-regulated within cardiomyocytes concomitant with cardiac hypertrophy. GATA5 is expressed in the embryo in the allantois, heart, lungs, gut, bladder and urogenital ridge, and in the adult animal, in the stomach and small intestines as well as bladder and lungs. GATA5 is described as a master regulator for cardiogenic pathways during development. In spite of this, GATA5 knockout animals are fully viable, with females showing developmental lesions in the urogenital tract. GATA6 is expressed within the embryo in the primitive streak, allantois, visceral endoderm, heart, vascular smooth muscle, lungs, gut and urogenital ridge. Null allele animals die early in gestation (e5.5-e7.5), due to defects in the visceral endoderm. In adult animals, GATA6 is expressed in the heart, aorta, stomach and small intestine, bladder as well as weakly in the liver and lung. It is suspected that functional redundancy exists between GATAs 4, 5 and 6.

Individual GATA proteins are able to confer tissue specificity of action by interacting with a large array of associated proteins, which are themselves expressed in tissue specific or semi-specific patterns. GATA proteins contribute to the formation of multi protein transcriptional complexes, by binding to such proteins as homeobox members, SOX members, Hand members, Friend of GATA (FOG) proteins, and even to themselves. Furthermore, tissue specific interactions can be important in defining tissue specific gene expression; for example, interactions between GATA4 and NKX2.5, MEF-2 and NFATc4 proteins can define myocardial gene expression.

Recently, GATA proteins, which are found in the nucleus, have been linked to membrane mediated cell signaling pathways (for example, Tremblay, 2003). For instance, GATA3 is linked to the TGFβ signaling pathway via phosphorylated SMAD3 protein, which translocates to the nucleus and interacts physically with GATA3. Target genes of GATA4 in the heart include many that are involved in the contractile machinery, and there is a link between cardiac myocyte hypertrophy and activation of GATA4 via the GTPase RhoA and the MAP-kinase pathway. The MAP-kinase pathway is also functional during development, with external stimulation via fibronectin; in fact, mice that are null allele for fibronectin show a very similar phenotype to those that are null allele for GATA4. Endothelin-1 signals, mediated through the Endothelin receptor and endothelin converting enzyme, make use of transcriptional complexes involving GATA4, homeodomain NK proteins and SRF proteins to mediate transcriptional responses. It is relevant to this discussion that the endothelin signaling pathway is associated with neural crest related developmental anomalies (see previous section).

In contrast to vertebrates, Drosophila GATA orthologs are known to be involved in the development of ectodermal structures. For example, in association with the Achaete/scute gene product, GATA homologues are functional in defining sensory and positional information. MASH1 is the vertebrate homologue for achaete/scute, involved in mammalian CNS and neural crest formation; mutations in MASH1 can result in developmental anomalies of sympathetic, parasympathetic and enteric ganglia.

GATA4 has been increasingly implicated as an important gene for the process of sex determination. Original expression studies have shown that GATA4 is expressed within the gonads (Viger 1998), and more recent studies have shown that the MIS gene promoter is activated by GATA4 (Tremblay and Viger 2001; Tremblay 2001) and that multiple gonadal promoters are dependant on GATA activity (Robert 2002).

Prior to the present invention, a direct and specific correlation between GATA4 gene expression and neural crest cell formation, migration, survival and differentiation had not been described.

Mutations in the GATA4 gene are not currently associated with neural tube defects or with congenital anomalies involving neural crest cells and their derivatives.

The Transcription Factor LHX9

LHX9 is a LIM homeodomain transcription factor whose expression is reported in the developing brain, dorsal neural tube, limb buds, gonads and pancreas (Retaux et. al. 1999; Birk et al. 2000). Genomic knockouts of the LHX9 gene in the mouse results in a viable phenotype displaying developmental failure of the gonads.

Prior to the present invention, a direct and specific correlation between LHX9 gene expression and neural crest cell formation, migration, survival and differentiation had not been described.

The Transcription Factor WT-1

Prior to the present invention, a direct and specific correlation between WT-1 gene expression and neural crest cell formation, migration, survival and differentiation had not been described.

The Transcription Factor DAX1

The transcription factor DAX1 is an orphan member of the nuclear hormone receptor family of transcription factors. The gene for DAX1 is located on the X chromosome, and mutations or duplications at this site result in developmental anomalies with the adrenal gland and with the sex organs (reviewed in Eyer and McCabe, 2004).

Prior to the present invention, a direct and specific correlation between DAX1 gene expression and neural crest cell formation and migration had not been described, nor had an expression of DAX1 within the developing pancreas nor had a developmental link between neural crest cells and the pancreas.

The Transcription Factor SRY

The SRY gene encodes a nuclear transcription factor containing an HMG box DNA binding motif. SRY is the Y chromosome located gene responsible for initiating the development of testes from the indifferent genital ridge, i.e. it is the mammalian testes determination factor (reviewed in Morrish and Sinclair, 2002).

Prior to the present invention, a direct and specific correlation between SRY gene promoter activity and neural crest cell formation, migration and differentiation had not been described, nor had the activity of the SRY promoter within the developing pancreas.

To their knowledge, the applicants are the first to have directly isolated living neural crest derived cells from dermal papillae, cells presumed to exist and postulated to be pluripotent (Sieber-Blum 2004a,b). This will greatly facilitate the characterization of these cells and the identification and isolation of their human orthologs. Their human orthologs could then be used in neuronal, endocrinal, cardiac, bone, muscle, teeth and auditory receptors applications. The present invention also provides means for identifying and isolating pancreatic progenitor cells from the mouse and thus provides means for characterizing the gene expression profile of these cells, to manipulate their development into B cells, and to identify, isolate and manipulate equivalent human pancreatic progenitor cells

More specifically, in accordance with the present invention, there is thus provided a vector comprising a promoter sequence driving the coding sequence of a visual marker protein, wherein the promoter sequence functions specifically in neural crest cells. In a more specific embodiment, the promoter sequence of this vector is a GATA4 promoter sequence. In an other more specific embodiment, the promoter sequence is a rat GATA4 promoter sequence as set forth in SEQ ID NO: 1. In an other more specific embodiment, the promoter sequence of this vector is a LHX9 promoter sequence. In an other more specific embodiment, the promoter sequence of this vector is a human LHX9 promoter sequence as set forth in SEQ ID NO: 2. In an other more specific embodiment, the promoter sequence of this vector is a WT-1 promoter sequence. In an other more specific embodiment, the promoter sequence of this vector is a human WT-1 promoter sequence as set forth in SEQ ID NO: 3. In an other more specific embodiment, the promoter sequence of this vector is a DAX1 promoter sequence. In an other more specific embodiment, the promoter sequence of this vector is a pig DAX1 promoter sequence as set forth in SEQ ID NO: 4. In an other more specific embodiment, the promoter sequence of this vector is a SRY promoter sequence. In an other more specific embodiment, the promoter sequence of this vector is a pig SRY promoter sequence as set forth in SEQ ID NO: 5. In an other more specific embodiment, the visual marker protein is a fluorescent protein. In an other more specific embodiment, the fluorescent protein is selected from the groups consisting of Green fluorescent protein (GFP), Cyanin fluorescent protein (CyaninFP), Yellow fluorescent protein (YellowFP), Blue fluorescent protein (BlueFP) and Red fluorescent protein (RedFP)

In accordance with the present invention, there is also provided a recombinant host cell comprising a vector of the present invention. In accordance with the present invention, there is also provided a recombinant cell population comprising a cell of the present invention.

In accordance with the present invention, there is also provided a transgenic non human animal comprising a recombinant host cell of the present invention.

In accordance with the present invention, there is also provided a method of observing neural crest cells activity comprising generating a transgenic non human animal having cells which comprise a promoter sequence functioning specifically in neural crest cells, and wherein the promoter drives the expression of a visual marker protein, whereby expression of the marker protein in the transgenic non human animal denotes neural crest cells activity. In a more specific embodiment of this method, the promoter is selected from the group consisting of GATA4, LHX9, WT-1, DAX1 and SRY.

In accordance with the present invention, there is also provided a method of isolating neural crest cells comprising: producing a transgenic non-human animal of the present invention; dissecting tissues known to contain neural crest cells, and isolating cells expressing the marker protein from said tissues, whereby cells expressing the marker protein in the dissected tissues are neural crest cells. In accordance with the present invention, there is also provided a cell isolated through this method. There is also provided a cell population derived from a cell isolated through this method.

In accordance with the present invention, there is also provided a method of identifying genes expressed in neural crest cells comprising assaying cells of the present invention for gene expression.

In accordance with the present invention, there is also provided a method of isolating pancreatic cells comprising: producing a transgenic non-human animal of the present invention; isolating the region of the animal known to contain pancreatic tissue from the remainder of the animal to yield a dissected pancreatic region, and isolating cells expressing the marker protein from said dissected pancreatic region, whereby cells expressing the marker protein in the dissected pancreatic region are pancreatic cells. There is also provided a cell isolated through this method and a cell population derived from such cell. In accordance with the present invention, there is also provided a method of identifying genes expressed in pancreatic cells comprising assaying cells isolated with the above-cited method for gene expression

In accordance with the present invention, there is also provided a method for identifying a biological agent which affects proliferation, differentiation or survival of neural crest cells or pancreatic cells, comprising: (a) comparing the proliferation, differentiation or survival of a cell population of the present invention in the absence and in the presence of a candidate biological agent, wherein the candidate biological agent is selected when the proliferation, differentiation or survival of said cell population differs in the absence and in the presence of said candidate biological agent. In a more specific embodiment of this method, step (a) comprises determining the effects of said candidate biological agent on differentiation of said cell population. In an other more specific embodiment of this method, wherein step (a) comprises determining the effects of said candidate biological agent on the proliferation of said cell population. In a more specific embodiment, the method further comprises the step of inducing differentiation of said cell population prior to performing step (a). In a more specific embodiment of this method, said biological agent is a growth factor selected from the group consisting of FGF-1, FGF-2, EGF, EGF-like ligands, TGF.alpha., IGF-1, NGF, PDGF, TGFβs and bFGF. In a more specific embodiment of this method, said cell population consists essentially of the progeny of a single of said fluorescent cells.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals (Sambrook 1989; Ausubel 1994).

### DEFINITIONS

The present description refers to a number of routinely used terms, including recombinant DNA (rDNA) technology terms. Nevertheless, definitions of selected examples of such terms are provided for clarity and consistency.

Transgenic Animals

As used herein, the terminology “transgenic non human animal” refers to any non human animal which harbors a nucleic acid sequence having been inserted into a cell and having become part of the genome of the animal that develops from that cell. In one specific embodiment of the present invention, the genetic alteration of the transgenic non human animal has been introduced in a germ-line cell, such that it enables the transfer of this genetic alteration to the offspring thereof. Such offspring, containing this genetic alteration are also transgenic non human animals.

Techniques for the preparation of such transgenic animals are well known in the art (e.g. a standard pronuclear microinjection (Hogan 1994); introduction of a transgene in embryonic stem (ES) cells; microinjecting the modified ES cells into blastocyst; or infecting a cell with a recombinant virus containing the transgene in its genome). Non-limiting examples of patents relating to a transgenic non-human animal include U.S. Pat. Nos. 4,736,866; 5,087,571; 5,175,383; 5,175,384 and 5,175,385. Many animals may be used as host for the transgenes of the present invention, including all laboratory animals including mice, rats and rabbits. In a specific embodiment, the transgenic animal is a mouse. In a more specific embodiment, the mouse strain is FVB/N. This mouse strain being albino may advantageously be used to enable the use of a tyrosinase minigene as a pigmentation marker of transgenesis (Methot 1995). Any other mouse strain however may be used in accordance with the present invention and identified as containing the transgene with the GFP fluorescence or other fluorescence. Other commonly used mouse strains for transgenic studies include C57Black, CD1 and ICR.

Promoters

As used herein, the term “GATA4 promoter” refers to any GATA4 promoter that may be used to stably express a marker protein such as a fluorescent protein in a transgenic non human animal according to the present invention so as to provide a reliable visual marker for neural crest cells and pancreatic cells. It includes any vertebrate GATA4 promoter. In more specific embodiments, the GATA4 promoter is a mammalian promoter such as a mouse, rat or human promoter. Means for identifying such other useful promoters in other animal species is described in Example 18 below.

As used herein, the term “LHX9 promoter” refers to any LHX9 promoter that may be used to stably express a marker protein such as a fluorescent protein in a transgenic non human animal according to the present invention so as to provide a reliable visual marker for neural crest cells. It includes any vertebrate LHX9 promoter. In more specific embodiments, the LHX9 promoter is a mammalian promoter such as a mouse, rat or human promoter. Means for identifying such other useful promoters in other animal species is described in Example 18 below.

As used herein, the term “WT-1 promoter” refers to any WT-1 promoter that may be used to stably express a marker protein such as a fluorescent protein in a transgenic non human animal according to the present invention so as to provide a reliable visual marker for neural crest cells. It includes any vertebrate WT-1 promoter. In more specific embodiments, the WT-1 promoter is a mammalian promoter such as a mouse, rat or human promoter. Means for identifying such other useful promoters in other animal species is described in Example 18 below.

As used herein, the term “DAX1 promoter” refers to any DAX1 promoter that may be used to stably express a marker protein such as a fluorescent protein in a transgenic non human animal according to the present invention so as to provide a reliable visual marker for neural crest cells and pancreatic cells. It includes any vertebrate DAX1 promoter. In more specific embodiments, the DAX1 promoter is a mammalian promoter such as a mouse, rat or human promoter. Means for identifying such other useful promoters in other animal species is described in Example 18 below.

As used herein, the term “SRY promoter” refers to any SRY promoter that may be used to stably express a marker protein such as a fluorescent protein in a transgenic non human animal according to the present invention so as to provide a reliable visual marker for neural crest cells and pancreatic cells. It includes any vertebrate SRY promoter. In more specific embodiments, the SRY promoter is a mammalian promoter such as a mouse, rat or human promoter. Means for identifying such other useful promoters in other animal species is described in Example 18 below.

Cells

As used herein, and unless otherwise provided, the term “neural crest cells” is meant to include “neural crest cell derivatives” as well as “neural crest stem cells”.

As used herein, the term “neural crest cell derivatives” is meant to refer to tissues and structures originating from the migration and differentiation of neural crest cells. Without being so limited this term includes tissues and structures described in Table 1 below.

As used herein, the term “neural crest stem cells” is meant to refer to neural crest cells that are pluripotential/multipotent in their developmental capacities. A neural crest stem cell is an undifferentiated neural crest cell that can be induced to proliferate using methods known in art. The neural crest stem cell is capable of self-maintenance, meaning that with each cell division, at least one daughter cell will also be a stem cell. The non-stem cell progeny of a neural crest stem cell are termed progenitor cells. The progenitor cells generated from a single multipotent neural crest stem cell are capable of differentiating into tissues as described in Table 1. Hence, the neural crest stem cell is “multipotent/pluripotent” because its progeny have multiple differentiative pathways.

As used herein, and unless otherwise provided, the term “pancreatic cells” are meant to include pancreatic progenitor cells in the developing pancreas, and in the mature pancreas, cells of the pancreatic ducts, the exocrine acini, and endocrine islets, including for the latter, A, B, D and pp cells.

As used herein, and unless otherwise provided, the term “pancreatic progenitor cells” are meant to include cells that are capable of differentiating into pancreatic ducts, the exocrine acini, and the endocrine islets, including for the latter, A, B, D and pp cells.

As used herein, the term “visual marker protein” refers to any marker protein that may be linked to a promoter of the present invention so as to be expressed in a neural crest cell of a transgenic non human animal and thereby create a living and real time visual marker for neural crest cells. In certain embodiments, the visual marker protein is also expressed in a pancreatic progenitor cell of a transgenic non human animal and thereby creates a living and real time visual marker for pancreatic progenitor cells.

As used herein, the term “fluorescent protein” is used to refer to any fluorescent protein that may be linked to a promoter of the present invention so as to be expressed in a neural crest cell and, in specific embodiments, in pancreatic progenitor cells of a transgenic non human animal and thereby create a living and real time visual marker for neural crest cells and, in specific embodiments, in pancreatic progenitor cells. Without being so limited, it is meant to include any fluorescent protein suitable for transgenic expression including Green fluorescent protein (GFP), Cyanin fluorescent protein (CyaninFP), Yellow fluorescent protein (YellowFP), Blue fluorescent protein (BlueFP) and Red fluorescent protein (RedFP) or variants thereof.

As used herein, the term “GATA4p.GFP marker” refers to a transgenic non human animal containing a transgene encoding a GATA4 promoter driving a green fluorescent protein. In a similar fashion, as used herein, the term “GATA4p.RFP marker” refers to a transgenic non human animal containing a transgene encoding a GATA4 promoter driving a red fluorescent protein. In a similar fashion, as used herein, the term “LHX9p.YFP marker” refers to a transgenic non human animal containing a transgene encoding a LHX9 promoter driving a yellow fluorescent protein. In a similar fashion, as used herein, the term “WT1p.GFP marker” refers to a transgenic non human animal containing a transgene encoding a WT-1 promoter driving a green fluorescent protein. In a similar fashion, as used herein, the term “DAX1p.YFP marker” refers to a transgenic non human animal containing a transgene encoding a DAX1 promoter driving a yellow fluorescent protein. In a similar fashion, as used herein, the term “SRYp.YFP marker” refers to a transgenic non human animal containing a transgene encoding a SRY promoter driving a yellow fluorescent protein.

TABLE 1
NEURAL CREST CELL TISSUE DERIVATIVES
(Adapted from Gray's Anatomy 38^{th} Edition (1995), p.145)

Neural Derivatives: (derived from cranial and troncal neural crest cells)
Sensory neurons of cranial ganglia V, VII, VIII, IX, X, including otic
sensory cells.
Sensory neurons of spinal dorsal root ganglia, plus peripheral sensory
receptors
Sympathetic ganglia (neurons, glia)
Parasympathetic ganglia (neurons, glia)
Enteric plexuses (neurons, glia)
Schwann cells of all peripheral nerves
Medulla of adrenal gland; Chromaffin cells; neuroendocrine cells
Carotid body cells
Melanocytes
Mesenchymal derivatives (derived from cranial neural crest cells):
Bones of head (frontal, parietal, nasal, palatine, maxillae, mandible, etc.)
Meninges
Choroid, schlera of eye
Connective tissue of lacrimal, nasal, labial, palatine, salivary glands
Dentine of teeth
Cartilage, ligaments, tendons of head
Connective tissue of thyroid gland, pharyngial pouches (parathyroid,
thymus)
Tunica media of outflow tract of heart, great vessels

As used herein, the term “cell lineage” refers to the derivation of a cell from the undifferentiated tissues of the embryo.

As used herein, the term “neural crest cell activity” refers to all neural crest cell activity including developmental processes that neural crest cells undergo including cell commitment, migration, division, survival and differentiation. It also includes gene expression in neural crest cells.

As used herein, the term “pancreatic cell activity” refers to all pancreatic cell activity including developmental processes that pancreatic cells undergo including cell commitment, migration, division, survival and differentiation. It also includes gene expression in pancreatic cells.

As used herein, the terminology “promoter that functions specifically in neural crest cells” refers to promoters which may function in other cell types provided that if they do, neural crest cells can nevertheless easily be isolated from these other cell types in transgenic animals. Without limiting the generality of the foregoing, these promoters include genes involved in neural crest cell activity namely GATA4, LHX9, WT-1, DAX1 and SRY. Hence, although the above-cited promoters function in neural crest cells, at least some of these also function in pancreatic cells. Neural crest cells can nevertheless be isolated from these other cells in transgenic animals according to the present invention by first separating neural crest cells tissues from pancreatic tissues by dissection and then by separating cells expressing marker proteins such as fluorescent proteins from surrounding cells that are not expressing marker proteins. In a specific embodiment, the promoter is selected from the group consisting of GATA4, LHX9, WT-1, DAX1 and SRY.

As used herein, the terminology “promoter that functions specifically in pancreatic cells” refers to promoters which may function in other cell types provided that if they do, pancreatic cells can nevertheless easily be isolated from these other cell types in transgenic animals. Without limiting the generality of the foregoing, these promoters include genes involved in pancreatic activity namely at least GATA4, DAX1 and SRY. Hence, while the above-cited promoters function in pancreatic cells, they also function in neural crest cells. Pancreatic cells can nevertheless be isolated from neural crest cells in transgenic animals according to the present invention by first separating pancreatic tissues from the other tissues by dissection and then by separating cells expressing marker proteins such as fluorescent proteins from surrounding cells that are not expressing marker proteins. In a specific embodiment, the promoter is selected from the group consisting of GATA4, DAX1 and SRY.

As used herein, the term “genes involved in neural crest cell activity” includes any gene that is expressed in neural crest cells or neural crest cell derivatives. Without being so limited this term includes GATA4, LHX9, WT-1, DAX1, SRY, EDNRb, Wnt1, PAX3, SOX10, KIT, KIT LIGAND, MASH1, MTHFR, EDN1, ECE1, RET, FGF8, PATCH, SHH, NTRK1, NEUROTROPIN, HAND2, GATA3, MITF, GDN, TISSUE PLASMINOGEN ACTIVATOR, Ppl and any other gene described above.

As used herein, the term “genes involved in pancreatic cell activity” includes any gene that is expressed in pancreatic cells. Without being so limited this term includes GATA4, DAX1, SRY, PDX1, NGN3, BETA2, PAX4, MAFA, Nkx2.2, NKX6.1, PDX1, BRN4, MAFB and PAX6.

As used herein, the term “cell population” includes any group of cells derived from fluorescent cells isolated according to the present invention. For instance, known methods for growing neuronal stem cells could be adapted for the present invention.

Cell Isolation

According to specific embodiments of the present invention fluorescent cells from the GATA4, LHX9, WT-1, DAX1 and SRY fluorescent mice are isolated, purified and the gene expression profile of these cells studied. Fluorescent cells are purified from non fluorescent cells via dissection, tissue digestion, and then via cell sorting with fluorescent activated cell separation (FACS) techniques (Daneau 2002; Boyer 2002a,b; Boyer 2004). Purified cells, both fluorescent and non fluorescent, are used for RNA recovery. Gene expression analysis such as reverse transcriptase polymerase chain reaction (RT-PCR) is then performed on these RNA populations using primers for relevant genes such as GATA4, LHX9, WT-1, DAX1 and SRY as well as those known to be involved in neural crest cells development. Such genes include GATA4, LHX9, WT-1, EDNRb, Wnt1, PAX3, SOX10, KIT, KIT LIGAND, MASH1, MTHFR, EDN1, ECE1, RET, FGF8, PATCH, SHH, NTRK1, NEUROTROPIN, HAND2, GATA3, MITF, GDN, TISSUE PLASMINOGEN ACTIVATOR and Ppl. Any other gene or protein profiling method (such as those commercialized by Affimetrix™) may be used to characterize cells identified according to the present invention as follows.

Arrays

The present invention also relates in one of its aspects to arrays and their uses in methods of the present invention.

As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

As used herein “Nucleic acid library” or “Nucleic acid array” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

As used herein “solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

The present invention enables a comparison of the gene expression profile of neural crest cells (visually marked) and non neural crest cells (not visually marked) for a given tissue at given time point, and a comparison of the gene expression profile of neural crest cells (visually marked) of a given tissue at different time points. Gene expression profile can also be performed to compare the gene expression of neural crest cells from normal embryos with neural crest cells from embryos harboring genetic lesions or subjected to environmental insults. The cellular precision afforded by the transgenic marking of gene expression via visual markers provides a significant advantage compared to tissue-based methods.

Similarly, in specific embodiments, the present invention enables a comparison of the gene expression profile of pancreatic progenitor cells (visually marked in the dissected tissue) and non pancreatic progenitor cells (not visually marked in the dissected tissue) at given time point. Gene expression profile can also be performed to compare the gene expression of pancreatic progenitor cells from normal embryos with pancreatic progenitor cells from embryos harboring genetic lesions or subjected to environmental insults. The cellular precision afforded by the transgenic marking of gene expression via visual markers provides a significant advantage compared to tissue-based methods.

Any nucleic acid arrays for determining gene expression can be used in accordance with the present invention. For instance, such arrays include Affymetrix's GeneChip™, those based on short or longer oligonucleotide probes as well as cDNAs or polymerase chain reaction (PCR) products (Lyons 2003). Other methods include serial analysis of gene expression (SAGE), differential display, (Ding 2004) as well as subtractive hybridization methods (Scheel 2002), differential screening (DS), RNA arbitrarily primer (RAP)-PCR, restriction endonucleolytic analysis of differentially expressed sequences (READS), amplified restriction fragment-length polymorphisms (AFLP), total gene expression analysis (TOGA; Ahmed 2002), and massive parallel signature sequencing (MPSS; Pollock 2002).

The present invention also encompasses arrays to detect and/or quantify other biological polymers such as proteins. For instance, the present invention may encompass arrays for the detection and quantification of proteins encoded by the genes expressed including genes coding for cell surface proteins and antigens, in cells isolated through the method of the present invention and more particularly their human orthologs. Such arrays include protein micro- or macroarrays or gel technologies including high-resolution 2D-gel methodologies, possibly coupled with mass spectrometry (Lopez 2003).

The present invention also relate to methods for the determination of the level of expression of transcripts or translation product of a single gene. The present invention therefore encompasses any known method for such determination including real time PCR and competitive PCR (Lyons 2003), Northern blots, nuclease protection, plaque hybridization and slot blots (Ahmed 2002).

Isolation and Culture of Neural Crest Cells and Pancreatic Progenitor Cells

Dissections, FACS and RT-PCR may be performed at all stages of embryonic development, and depending on the age of the mice (embryo, newborn or adult), different tissues may be observed. Hence, pre-migratory neural crest cells are best observed at day e8.5; migratory neural crest cells, including cranial neural crest, namely branchial arches at day e9.5-e10.5); troncal neural crest, namely the ventromedial pathway, at days e9.5-e10.5; dorsolateral pathway at days e12.5-e13.5; post migratory neural crest cells-trunk, including dorsal root, in newborn; sympathetic trunk in newborn; enteric nerve net at day e13.5 and in newborn; aorta at day e13.5 and in newborn; adrenal medulla at day e13.5 and in newborn; peripheral nerve Schwann cells in newborn; heart at day e13.5 and in newborn; olfactory bulbs in newborn and pituitary in adult. Pancreatic progenitor cells are best observed at e10.5 to e11.5.

Dissociated cells are centrifuged at low speed, between 200 and 2000 rpm, usually between 400 and 800 rpm, and then resuspended in culture medium. The neural crest cells can be cultured in suspension or on a fixed substrate. However, substrates tend to induce differentiation of the neural crest stem cell progeny. Thus, suspension cultures are preferred if large numbers of undifferentiated neural crest stem cell progeny are desired. Cell suspensions are seeded in any receptacle capable of sustaining cells, particularly culture flasks, culture plates or roller bottles, and more particularly in small culture flasks such as 25 cm² culture flasks. Cells cultured in suspension are resuspended at approximately 5×10⁴ to 2×10⁵ cells/ml, preferably 1×10⁵ cells/ml. Cells plated on a fixed substrate are plated at approximately 2-3×10³ cells/cm², preferably 2.5×10³ cells/cm².

The dissociated neural crest cells and pancreatic progenitor cells can be placed into any known culture medium capable of supporting cell growth, including HEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. However, a preferred embodiment for proliferation of neural crest stem cells is to use a defined, serum-free culture medium, as serum tends to induce differentiation and contains unknown components (i.e. is undefined). A defined culture medium is also preferred if the cells are to be used for transplantation purposes. A particularly preferable culture medium is a defined culture medium comprising a mixture of DMEM, F12, and a defined hormone and salt mixture. A culture as that described in U.S. Pat. No. 6,479,283 and 5,961,972, example 1 can be used to proliferate and/or induce differentiation of pancreatic cells.

Conditions for culturing should be close to physiological conditions. The pH of the culture medium should be close to physiological pH, preferably between pH 6-8, more preferably between about pH 7 to 7.8, with pH 7.4 being most preferred. Physiological temperatures range between about 30° C. to 40° C. Cells are preferably cultured at temperatures between about 32° C. to about 38° C., and more preferably between about 35° C. to about 37° C.

The culture medium is supplemented with at least one proliferation-inducing growth factor. As used herein, the term “growth factor” refers to a protein, peptide or other molecule having a growth, proliferative, differentiative, or trophic effect on neural crest stem cells and/or neural crest stem cell progeny. Growth factors which may be used for inducing proliferation include any trophic factor that allows neural crest stem cells and precursor cells to proliferate, including any molecule which binds to a receptor on the surface of the cell to exert a trophic, or growth-inducing effect on the cell. Preferred proliferation-inducing growth factors include EGF, amphiregulin, acidic fibroblast growth factor (aFGF or FGF-1), basic fibroblast growth factor (bFGF or FGF-2), transforming growth factor alpha (TGF.alpha.), and combinations thereof.

Preferred proliferation-inducing growth factors include EGF and TGF.alpha. A preferred combination of proliferation-inducing growth factors is EGF or TGF.alpha. with FGF-1 or FGF-2. Growth factors are usually added to the culture medium at concentrations ranging between about 1 fg/ml to 1 mg/ml. Concentrations between about 1 to 100 ng/ml are usually sufficient. Simple titration experiments can be easily performed to determine the optimal concentration of a particular growth factor.

In addition to proliferation-inducing growth factors, other growth factors may be added to the culture medium that influence proliferation and differentiation of the cells including NGF, platelet-derived growth factor (PDGF), thyrotropin releasing hormone (TRH), transforming growth factor betas (TGFβs), insulin-like growth factor (IGF-1) and the like.

Methods for Screening Effects of Drugs on Neural Crest Cells or Pancreatic Progenitor Cells

Neural crest stem cell progeny cultured in vitro can be used for the screening of potential neurologically therapeutic compositions. These compositions can be applied to cells in culture at varying dosages, and the response of the cells monitored for various time periods. Physical characteristics of the cells can be analyzed by observing cell and neurite growth with microscopy. The induction of expression of new or increased levels of proteins such as enzymes, receptors and other cell surface molecules, or of neurotransmitters, amino acids, neuropeptides and biogenic amines can be analyzed with any technique known in the art which can identify the alteration of the level of such molecules. Similarly, pancreatic cells progeny can be used for the screening of potential therapeutic compositions for treating diabetes or other conditions involving pancreatic cells defects. These techniques include immunohistochemistry using antibodies against such molecules, or biochemical analysis. Such biochemical analysis includes protein assays, enzymatic assays, receptor binding assays, enzyme-linked immunosorbant assays (ELISA), electrophoretic analysis, analysis with high performance liquid chromatography (HPLC), Western blots, and radioimmune assays (RIA). Nucleic acid analysis such as Northern blots can be used to examine the levels of mRNA coding for these molecules, or for enzymes which synthesize these molecules.

Alternatively, cells treated with these pharmaceutical compositions can be transplanted into an animal, and their survival, ability to form neural crest-derived tissues, or pancreatic tissues such as insulin producing cells and biochemical and immunological characteristics examined as previously described.

The ability to isolate purified neural crest cells from different tissues at different times of development is useful for ablation-transplantation type experiments in the mouse to test neural crest cell potential and commitment, at the cell level as opposed to the tissue level. Similarly, the ability to isolate purified pancreatic cells at different times of development is useful for ablation-transplantation type experiments in the mouse to test pancreatic cell potential and commitment, at the cell level as opposed to the tissue level.

The present description refers to a number of documents, the content of which is herein incorporated by reference in their entirety.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents a mouse embryo neural crest expression pattern with an embryonic day 10.5 transgenic mouse embryo comprising the GATA4 promoter driving the GFP transgene according to one embodiment of the present invention. Left panels A (oriented to show the side of the whole embryo, 20×) and C (oriented to show the back, 40×) show the embryo under visible light while the right panels B and D present the same embryo as in panels A and C, respectively, with GFP filters to show neural crest cells location. It may be seen that fluorescence marks cranial and troncal neural crest cells (Panel B) and the segmented dorsal ganglia of the troncal neural crest while it is absent from the neural tube (Panel D);

FIG. 2 shows other parts of the transgenic mouse embryo (from the same line as that in FIG. 1) after 13.5 days of gestation. Left panels E and G show the embryo's torso at 20×, and abdominal cavity with liver and intestines removed at 32×, respectively under visible light, while right panels F and H present the embryo, of panels E and G, respectively, with GFP filters to show neural crest cells location. It may be seen that fluorescence marks the peripheral nerve branching in torso and limbs, and the outflow track of heart (Panel F) and the segmented troncal nerve tracks as well as testicular cords (Panel H);

FIG. 3 shows other parts of a transgenic mouse embryo (from the same line as that in FIG. 1) after 12.5 to 13.5 days of gestation. Left panel I shows the 12.5 days old embryo's stomach and intestine at 50× under visible light while panel K shows the 13.5 days old embryo's front limb at 40× under visible light. The right panels J and L present the embryo, of panels I and K respectively, with GFP filters to show neural crest cells location. It may be seen that fluorescence marks cells contributing to intestinal nerve network (Panel J) and the developing nerve tracks of limb (Panel L);

FIG. 4 presents a mouse embryo neural crest expression pattern with a 11.5 day old transgenic mouse embryo comprising the GATA4 promoter driving the RFP transgene according to one embodiment of the present invention. Left panels A (oriented to show the side of the whole embryo, 20×) and C (oriented to show the back, 40×) show the embryo under visible light while the right panels B and D present the same embryo as in panels A and C, respectively, with RFP filters to show neural crest cells location. It may be seen that fluorescence marks cranial and troncal neural crest cells (Panel B) and the segmented dorsal ganglia of the troncal neural crest, while it is absent from the neural tube (Panel D);

FIG. 5 presents a mouse embryo neural crest expression pattern with a 11.5 day old transgenic mouse embryo comprising the LHX9 promoter driving the YFP transgene according to one embodiment of the present invention. Left panels A (oriented to show the side of the whole embryo, 20×) and C (oriented to show the back, 32×) show the embryo under visible light, while the right panels B and D present the same embryo as in panels A and C, respectively, with YFP filters to show neural crest cells location. It may be seen that fluorescence marks cranial and troncal neural crest cells (Panel B) and the segmented dorsal ganglia of the troncal neural crest while it is absent from the neural tube (Panel D);

FIG. 6 presents a mouse embryo neural crest expression pattern with a 11.5 day old transgenic mouse embryo comprising the WT-1 promoter driving the GFP transgene according to one embodiment of the present invention. Left panels A (oriented to show the side of the whole embryo, 16×) and C (oriented to show the back, 40×) show the embryo under visible light, while the right panels B and D present the same embryo as in panels A and C, respectively, with GFP filters to show neural crest cells location. It may be seen that fluorescence marks cranial and troncal neural crest cells (Panel B) and the segmented dorsal ganglia of the troncal neural crest while it is absent from the neural tube (Panel D);

FIG. 7 presents a neural crest cell expression pattern in a skin of a 5 days old newborn transgenic mouse comprising the GATA4 promoter driving the RFP transgene according to one embodiment of the present invention. Left panels A (cross section of skin showing epidermis (ep), dermis (de), hypodermis (hy), hair shafts and dermal papillae (*), 80×) and C (bottom view of the skin sample hypodermal surface, 80×) shows the skin sample under visible light, while the right panels B and D present the same skin sample as in panels A and C, respectively, with RFP filters to show neural crest cells location;

FIG. 8 presents a neural crest cell expression pattern in a skin sample of a 5 days old newborn transgenic mouse comprising the LHX9 promoter driving the YFP transgene according to one embodiment of the present invention. Left panels A (cross section of skin showing epidermis (ep), dermis (de), hypodermis (hy), hair shafts and dermal papillae (*), 80×) and C (bottom view of the skin sample hypodermal surface, 80×) shows the skin sample under visible light, while the right panels B and D present the same skin sample as in panels A and C, respectively, with YFP filter to show neural crest cells location;

FIG. 9 presents an adult mouse neural crest expression pattern in a skin sample of a 10-week-old (adult) transgenic mouse comprising the LHX9 promoter driving the YFP transgene according to one embodiment of the present invention. Panels A (cross section of skin showing epidermis (ep), dermis (de), hypodermis (hy), hair shafts and dermal papillae (*), 100×) and C (cross section of skin showing dermal papillae, 400×) shows the skin sample under visible light, while the panels B and D present the same skin sample as in panels A and C, respectively, with YFP filter to show neural crest cells location. Panel E presents a merged image of panels C and D;

FIG. 10 presents a neural crest cell expression pattern in a skin sample of a 5 day old transgenic mouse comprising the WT-1 promoter driving the GFP transgene according to one embodiment of the present invention. Left panels A (cross section of skin showing epidermis (ep), dermis (de), hypodermis (hy), hair shafts and dermal papillae (*), 80×) and C (bottom view of the skin sample hypodermal surface, 80×) shows the skin sample under visible light, while the right panels B and D present the same skin sample as in panels A and C, respectively, with GFP filter to show neural crest cells location;

FIG. 11 graphically presents results of a FACS isolation of neural crest cells from surrounding tissue cells of four 300 □M slices of a dissected skin sample of a 5 day old mouse;

FIG. 12 presents a DAXpYFP positive mouse embryo of gestational age e11.5 days at 20× magnification. Image A shows the embryo under normal visible light illumination. Image B shows the same embryo with YFP fluorescence illumination. A pattern of fluorescence recognizable as representing neural crest cells in migration is readily observable. The red arrow indicates the location of the developing pancreatic tissue, which at this age can be detected via fluorescence from the exterior of the embryo;

FIG. 13 presents the developing pancreas from gestational age e15.5 day mouse embryos. Image A shows an 8× magnification of non transgenic (left) and DAXpYFP positive dissected tissues, including intestines and liver, as viewed from the caudal direction using visible light illumination. The blue arrows indicate the location of the developing pancreas. Image B represents the same tissues with YFP fluorescence illumination, with the transgenic pancreas displaying evident fluorescence. Image C shows a 32× magnification of the transgenic tissues seen in image A under normal visible light illumination; the blue arrow indicates the main pancreatic lobe while the blue arrowhead indicates the accessory pancreatic lobe. Image D shows the same tissues as image C under YFP fluorescence illumination, with the developing transgenic pancreas now displaying a lobular structure via evident fluorescence. Image E is a higher magnification (100×) fluorescent view of the main pancreatic lobe from images C and D, showing details of the lobular structure of the tissues;

FIG. 14 presents the pancreas dissected from an age e13.5 embryo of the rGATA4RFP transgenic mouse line, at 63× magnification. A. Dissected tissues as seen with visible light, where st denotes the stomach, sp denotes the spleen, v1p denotes the ventral lobe of the embryonic pancreas and d1p denotes the dorsal lobe of the embryonic pancreas. B. Same tissues as in A, as seen with fluorescence illumination and RFP filters. Foci of fluorescence can be readily seen within both the ventral and dorsal pancreatic lobes. The stomach also shows fluorescence (i.e. neural crest cells contribute to the neural network of stomach), while the spleen does not;

FIG. 15 presents a day e13.5 embryo and tissues from the pSRYp(2.6 kb)YFP transgenic mouse line. A. Whole embryo as seen with visible light, at 12.5× magnification. B. The same embryo as in A, as seen with fluorescence illumination and YFP filters. The fluorescence pattern marking the neural crest cells in migration is readily evident. C. Dissected pancreatic tissues taken from embryo in A, at 80× magnification as seen with visible light. In this image, int denotes a segment of intestine, sp denotes the spleen, vlp denotes the ventral lobe of the embryonic pancreas while dlp denotes the dorsal lobe of the embryonic pancreas. D. Same tissues presented in C, with fluorescence illumination and YFP filters. The dorsal and ventral lobes of the pancreas show fluorescence, as does the segment of the intestine but not the spleen. E. Dissected ventral lobe of pancreas from image C, flattened with a coverslip (squash preparation), using DIC optics and at 100× magnification. F. Same tissues as in E, viewed with fluorescence illumination and YFP filters. Foci of fluorescent cells as well as individual fluorescent cells are readily apparent;

FIG. 16 presents the nucleotide sequence of a rat GATA4 promoter (SEQ ID NO: 1);

FIG. 17 presents the nucleotide sequence of a human LHX9 promoter (SEQ ID NO: 2);

FIG. 18 presents the nucleotide sequence of a human WT-1 promoter (SEQ ID NO: 3);

FIG. 19 presents the nucleotide sequence of a pig DAX1 promoter (SEQ ID NO: 4);

FIG. 20 presents the nucleotide sequence of a pig SRY promoter (SEQ ID NO: 5)

FIG. 21 presents a comparison of the endogenous Gata4 mRNA expression in a e11.5 embryo (right panel) with the fluorescent profile of a e11.5 embryo from a Gata4p-GFP transgenic mouse line (left panel);

FIG. 22 presents the gene expression phenotyping of fluorescent neural crest cells isolated from the cranial (C) and troncal (T) tissues of a Gata4p-GFP embryo (embryo shown in panel A) through RT-PCR expression analysis of neural crest stem cell markers (panel B) and neural crest restricted fate markers (panel C);

FIG. 23 shows neural crest cells grown as spheres under conditions that favor stem cells proliferation under visible light (left panels) and GFP fluorescence (right panels);

FIG. 24 shows troncal crest cells grown as flattened colonies in conditions that allow cell differentiation (panel A) and a single differentiated cell with elongated, neuronal phenotype (shown with arrow in panel B); and

FIG. 25 shows troncal neural crest cells grown in conditions that allow cell differentiation and stained with antibodies against smooth muscle antigen, a marker for smooth muscle cells (left panels), and non specifically stained for nucleus (right panels).

### DESCRIPTION OF THE SPECIFIC EMBODIMENT

The present invention involves the use of promoters of genes specifically activated in neural crest cells linked to visual marker proteins so as to provide a visual marker for neural crest cells. The use of fluorescence according to specific embodiments of the present invention provides a vital and real time marker for developmental studies including FACS purification of fluorescent cell populations and expression profiling of these cells (Daneau 2002; Boyer 2002a) including subtractive hybridization studies (Boyer 2002b; Boyer 2004) and gene chip-based or other type of gene expression profiling. In more specific embodiments, the present invention also provides the use of promoters of genes specifically activated in developing pancreatic tissues linked to visual marker proteins so as to provide a visual marker for pancreatic progenitor cells.

The invention is based on the discovery that GATA4, LHX9, WT-1, DAX1 and SRY promoter-based marker transgenes are expressed specifically in neural crest cells. Indeed, most neural crest cells express these promoter-based transgenes during embryonic development, in the neonate and in the adolescent animal. As a consequence, most neural crest cells will fluoresce in an animal expressing in its cells a transgene of a promoter of any of these genes driving a fluorescent protein and the specific expression of these promoters will enable the isolation of neural crest cells from other tissues by dissecting neural crest containing tissues and then by isolating fluorescent cells from not fluorescent cells within the dissected tissues. The ubiquitous and specific expression pattern of these genes was confirmed by the observation that the fluorescence seen with the transgenic animals of the present invention follows known neural crest cells migration pathways.

The GATA4, LHX9, WT1, DAX1 and SRY transgenic animals of the present invention may therefore serve as model animals to evaluate the effect of various genetic defects on neural crest cell activity: animals presenting genetic defects can be mated with these model animals and their neural crest cells activity will be compared with that of the model animals. The transgenic animal markers also enable the pharmacological testing of animals under normal and disease situations, using analysis of neural crest cell function as a measurement of the effect of treatment. Similarly, these animals can serve as model animals to evaluate the effect of various genetic defects on pancreatic activity: animals presenting genetic defects can be mated with these model animals and their pancreatic cell activity will be compared with that of the model animals.

These markers also permit the isolation of neural crest cells at various times of the development of the animal and in various tissues of the animal and the analysis of the genetic expression profile of these cells. It also enables the isolation of neural crest stem cells for possible treatment of disorders including neurological disorders such as peripheral nerve disorders and sensory deafness, and certain endocrine disorders. Similarly, these markers permit the isolation of pancreatic cells at various times of the development of the animal and the analysis of the genetic expression profile of these cells. It also enables the isolation of pancreatic cells for possible treatment of disorders.

These markers also enable the isolation of neural crest stem cells for possible expansion and maintenance in an undifferentiated state in tissue culture. They enable the isolation of neural crest stem cells for possible controlled differentiation in tissue culture into cells displaying phenotypes of neurons, sensory neurons, glial cells, nerve receptor cells, heart cells including valves and septa, neuroendocrine cells, enteric nerve cells, muscle cells, bone cells, tendon cells, and adipose cells. Similarly, these markers also enable the isolation of pancreatic cells for possible expansion and maintenance in an undifferentiated state in tissue culture.

The present invention will now be described by way of non-limitative illustrative embodiments.

### EXAMPLE 1

Generation of Transgenic Mice

Transgenic mice were generated via standard pronuclear microinjection of the transgenes of interest (Hogan 1994). FVB/N female mice were used for embryo collection to aid in visual identification of transgenic animals, a tyrosinase minigene was co-injected with the transgene of interest (Methot 1995). Mice incorporating the transgene of interest were identified via the presence of fluorescence in their tissues, visible in newborn animals using a stereomicroscope equipped with epi-fluorescence.

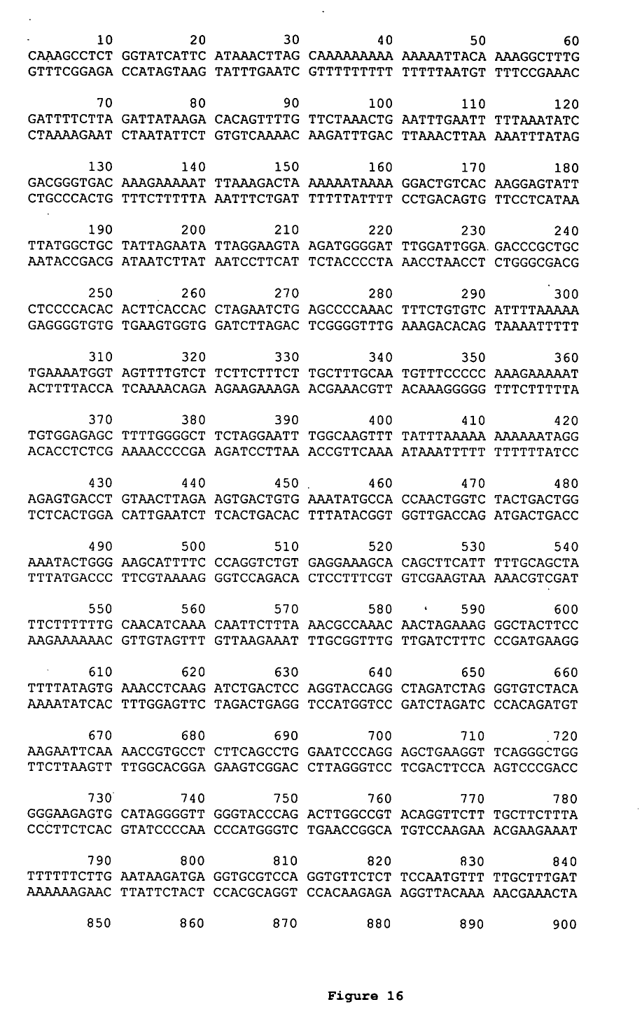

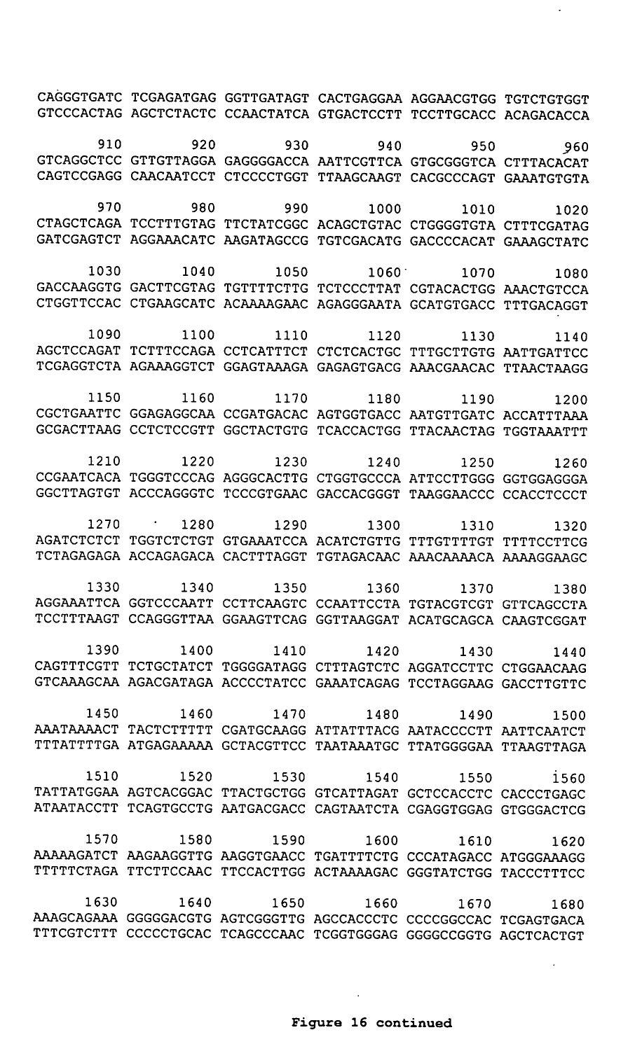

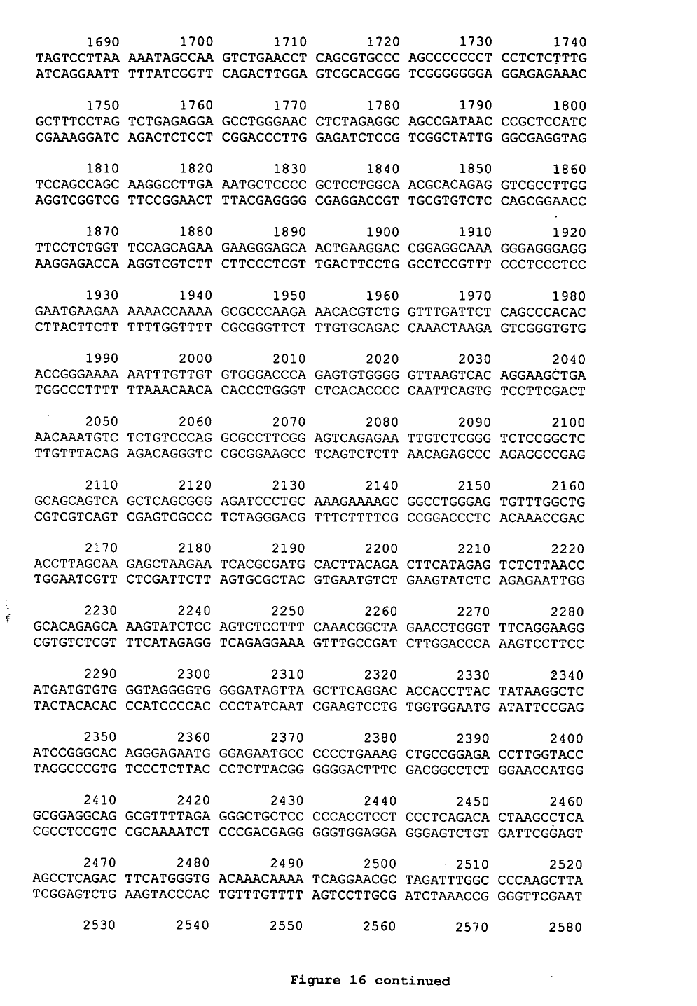

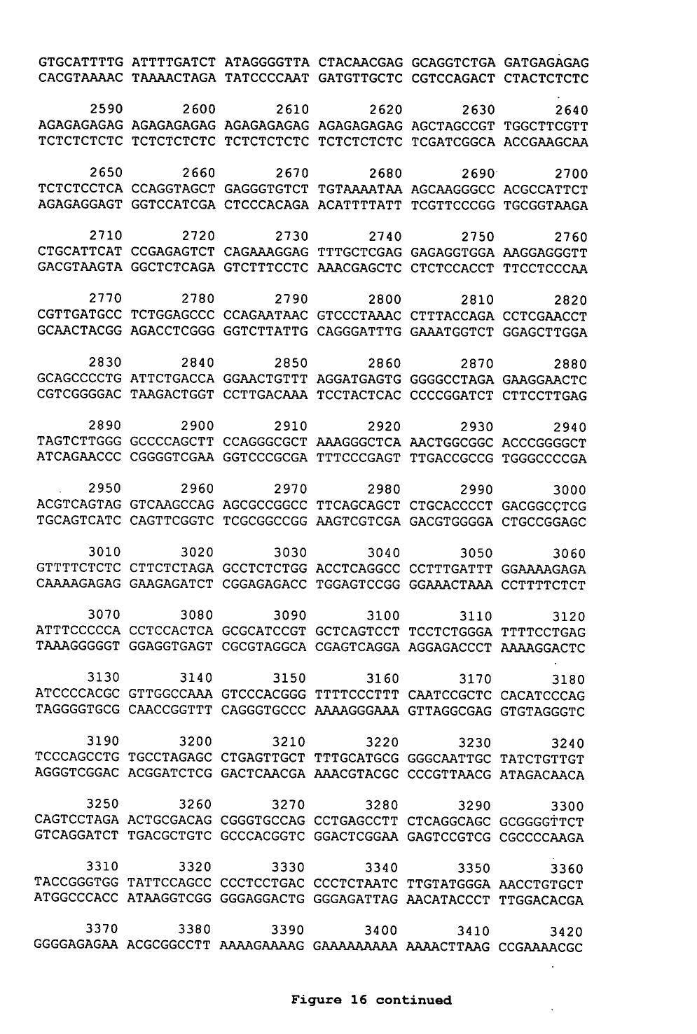

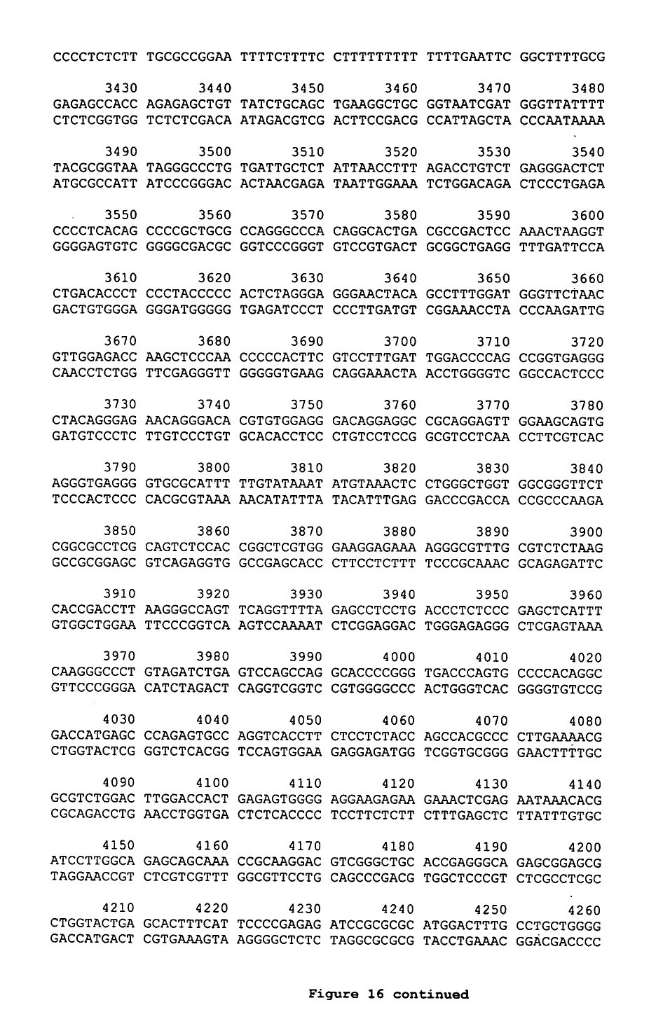

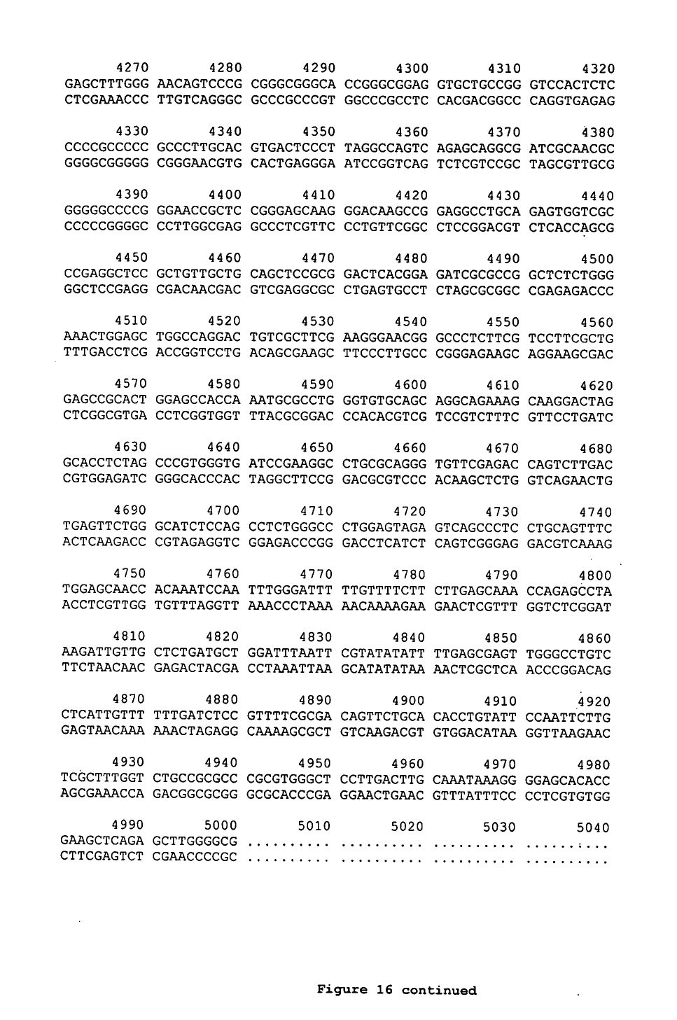

A transgene was formulated based on 5 Kb of rat GATA4 promoter sequences (excluding intron 1) driving the coding sequence of green fluorescent protein (GFP). A sequence size of 5 Kb was selected as a pragmatic compromise between a sequence long enough to insure proper expression, and short enough to manipulate with PCR and with plasmid vectors. At least a portion of rat GATA4 promoter sequence may be found in Genbank genomic databanks, by performing a blast search using mouse GATA4 sequences. These sequences were amplified by PCR from rat genomic DNA using standard molecular biology procedures. The promoter sequence used is presented in FIG. 16 (SEQ ID NO: 1). This transgene was microinjected into FVB/N embryos and six independent transgenic lines of mice were generated.

A transgene was also created based on the same 5 kb of rat GATA4 promoter sequences (excluding intron 1) driving the coding sequence of red fluorescent protein (RFP). This transgene was microinjected into FVB/N embryos and 4 independent lines of mice were generated.

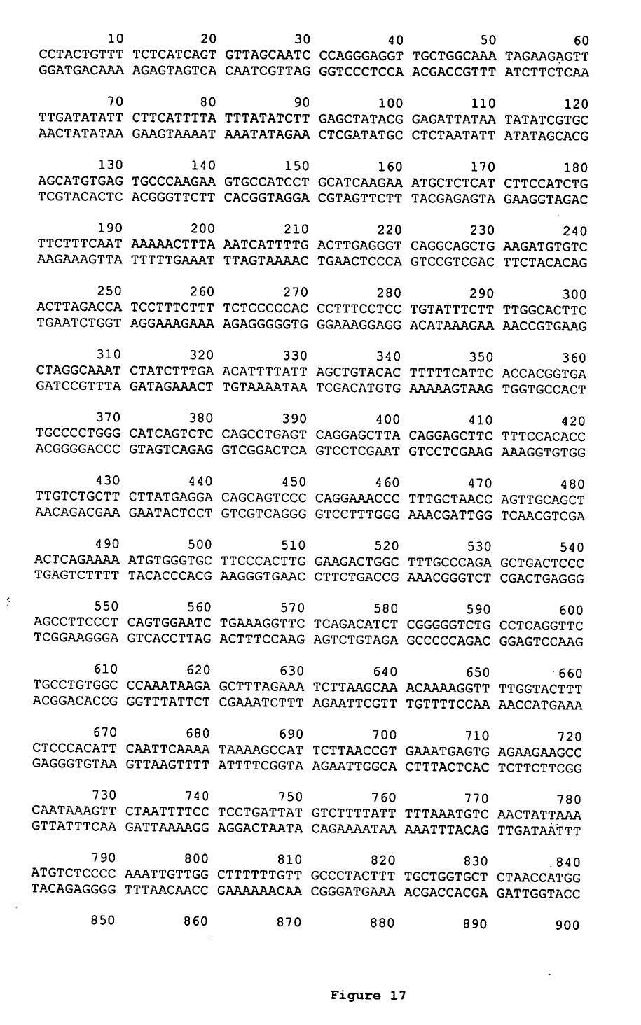

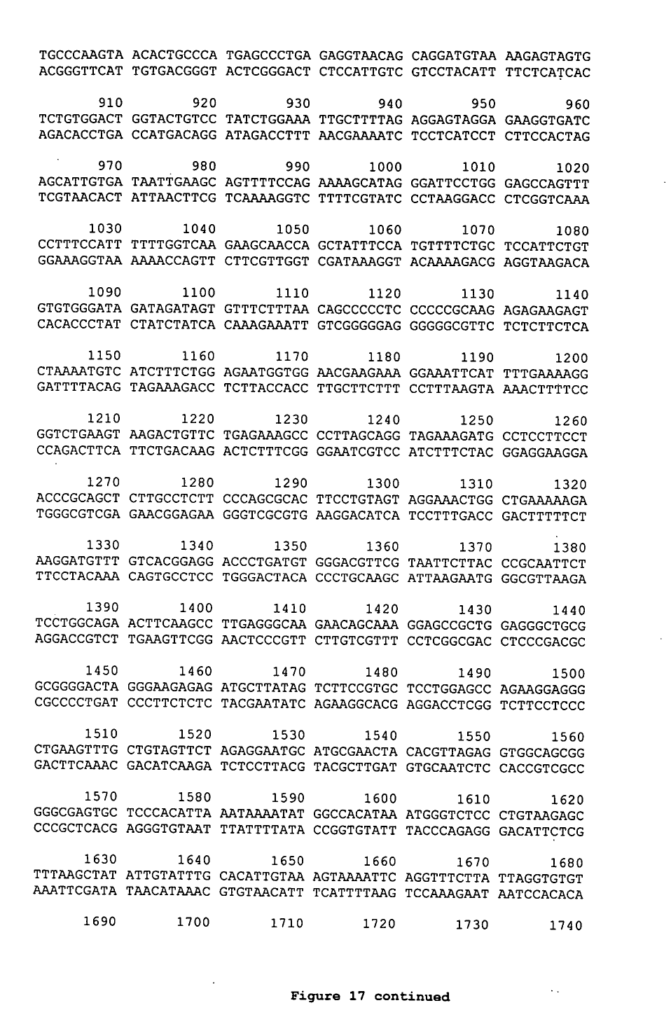

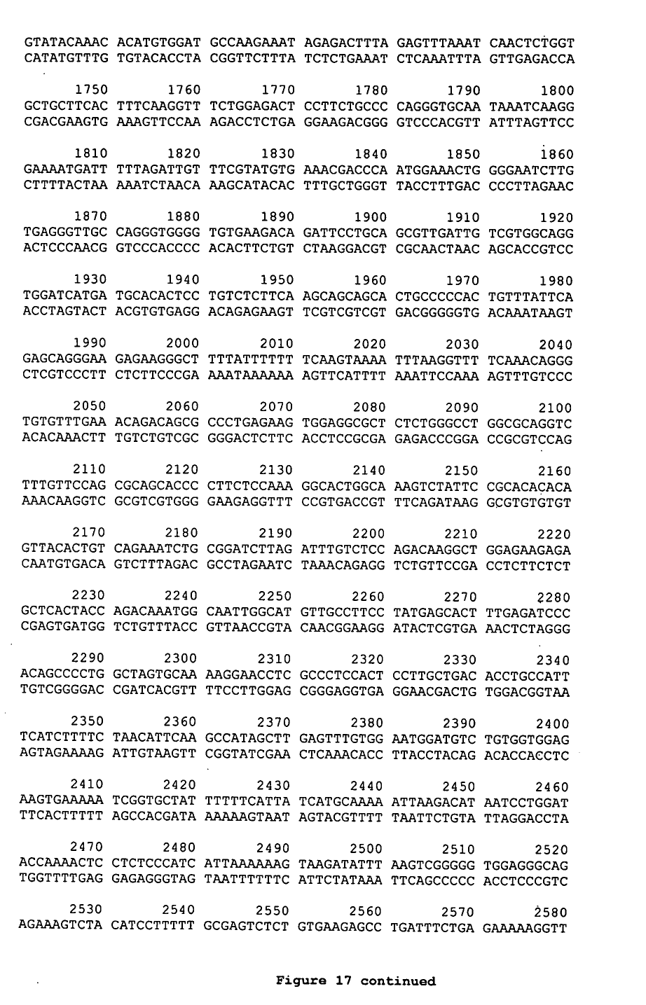

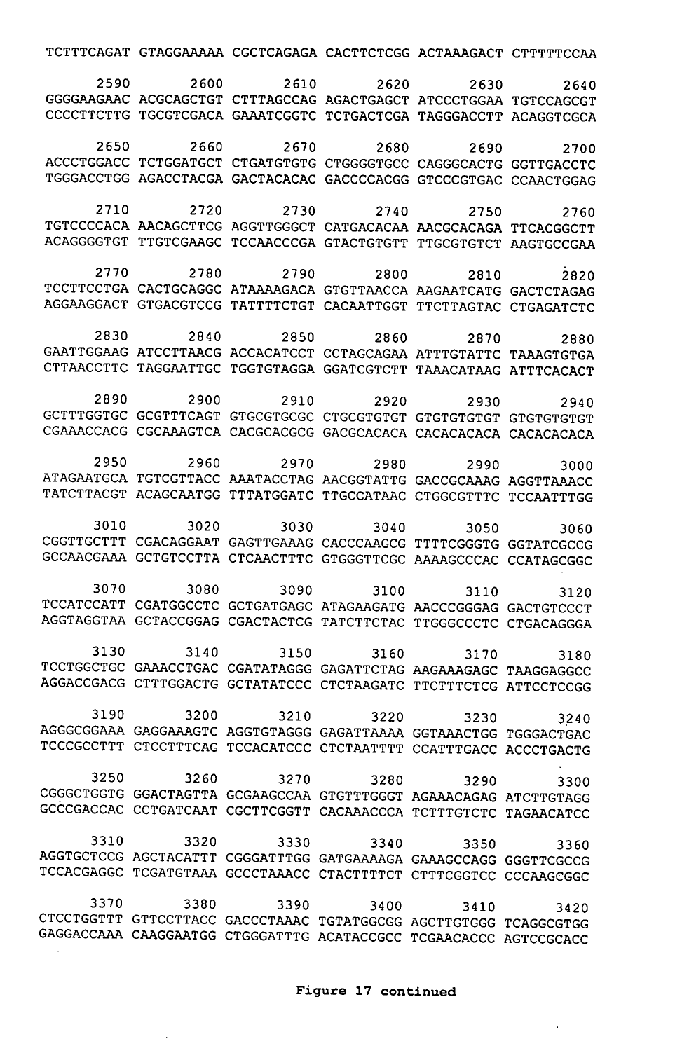

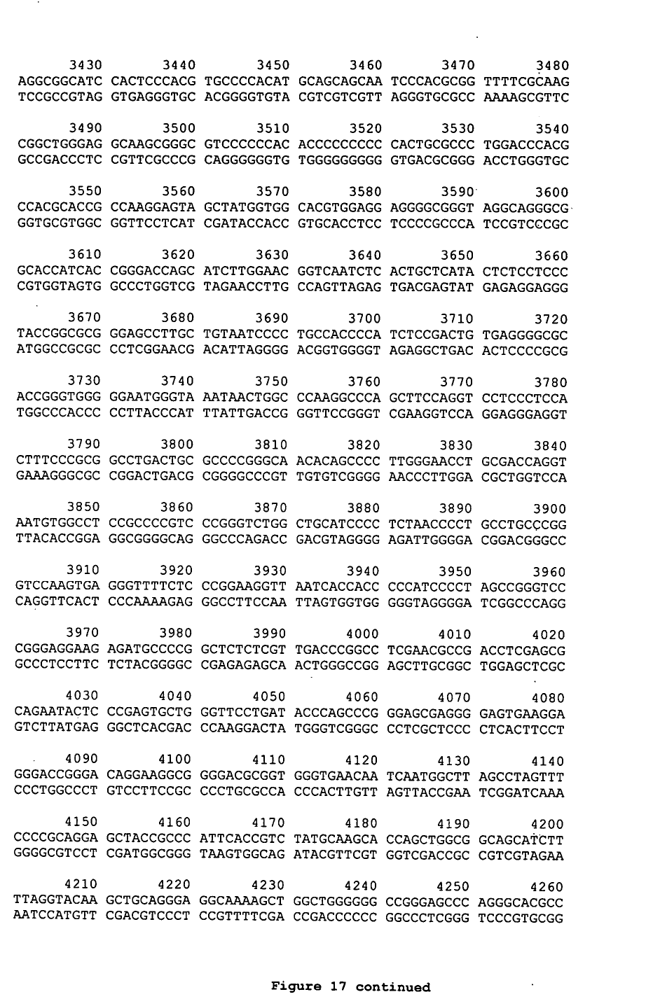

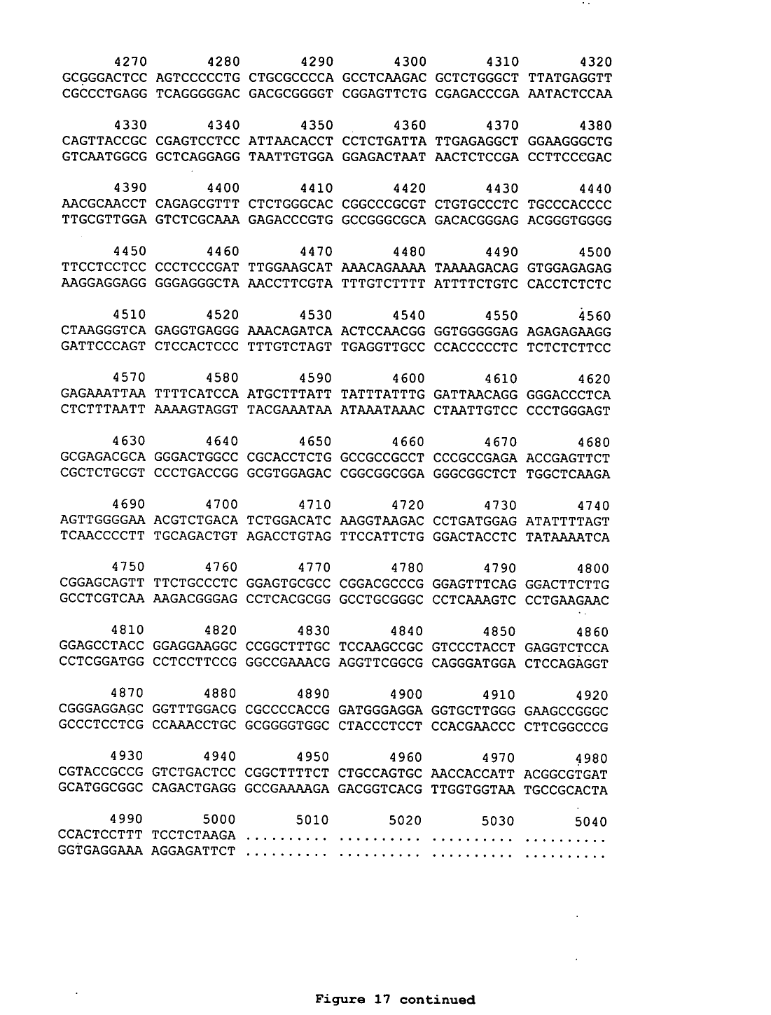

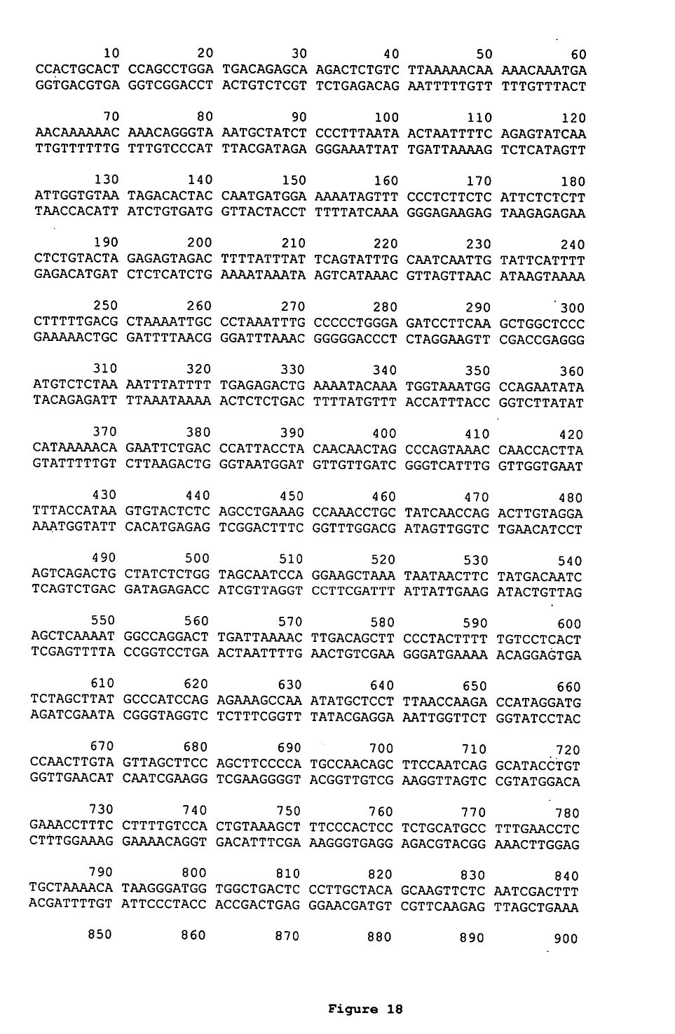

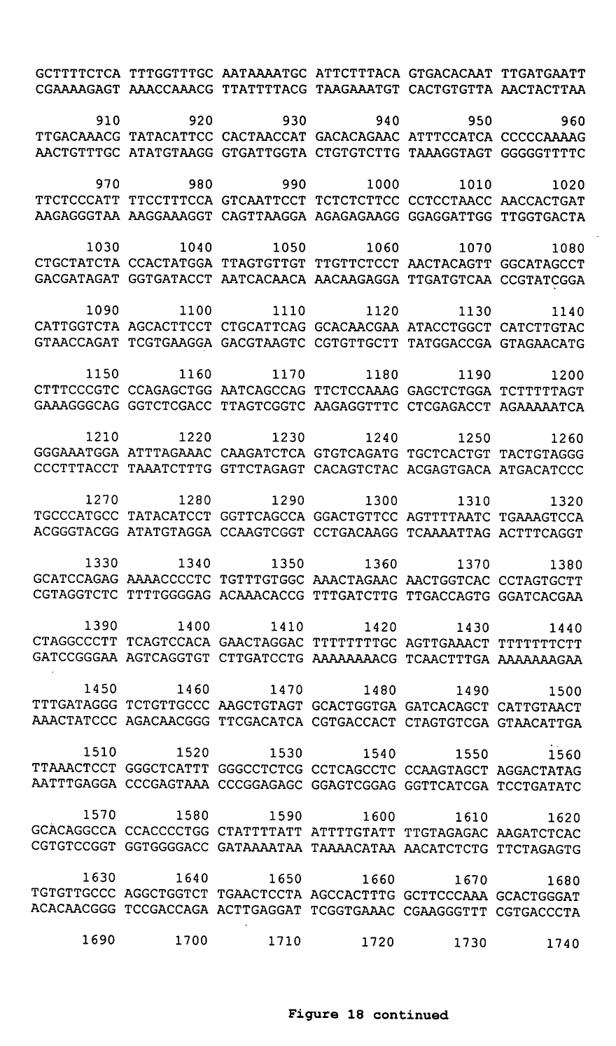

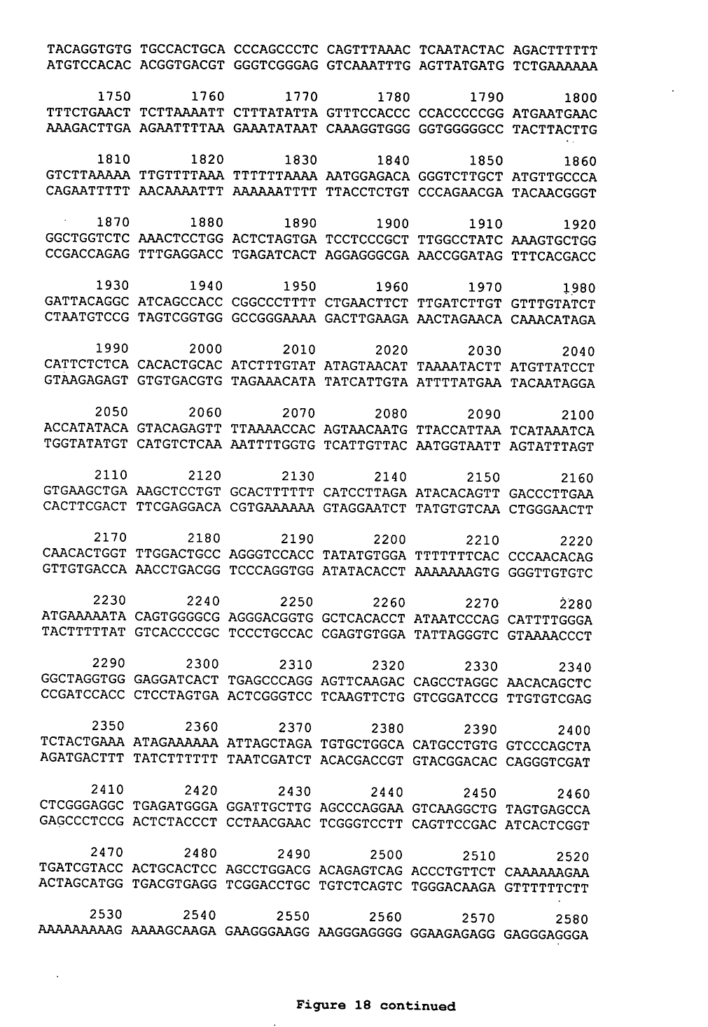

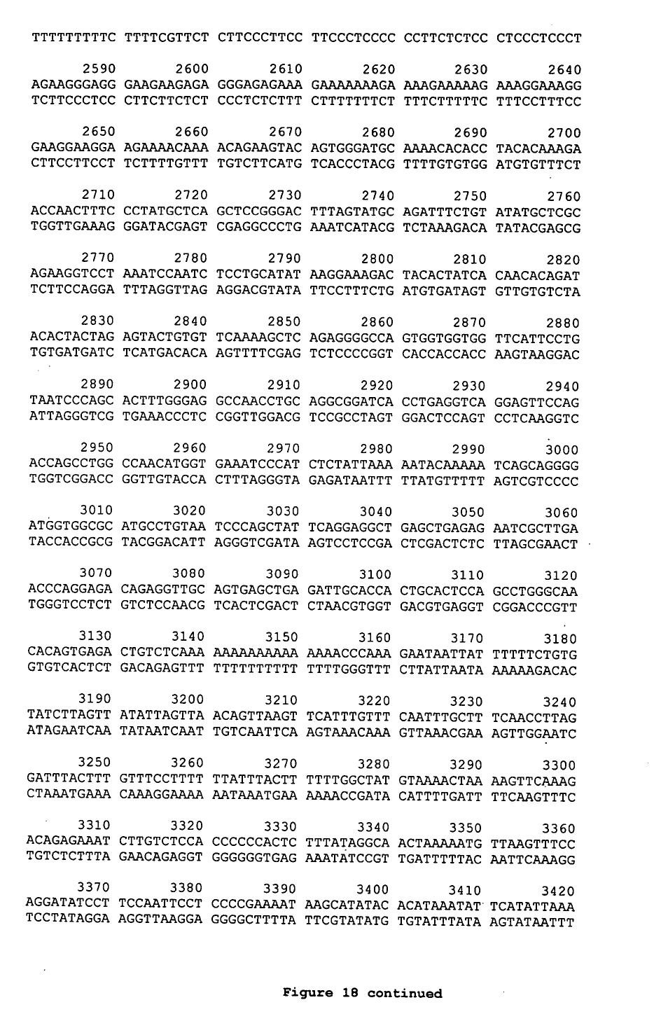

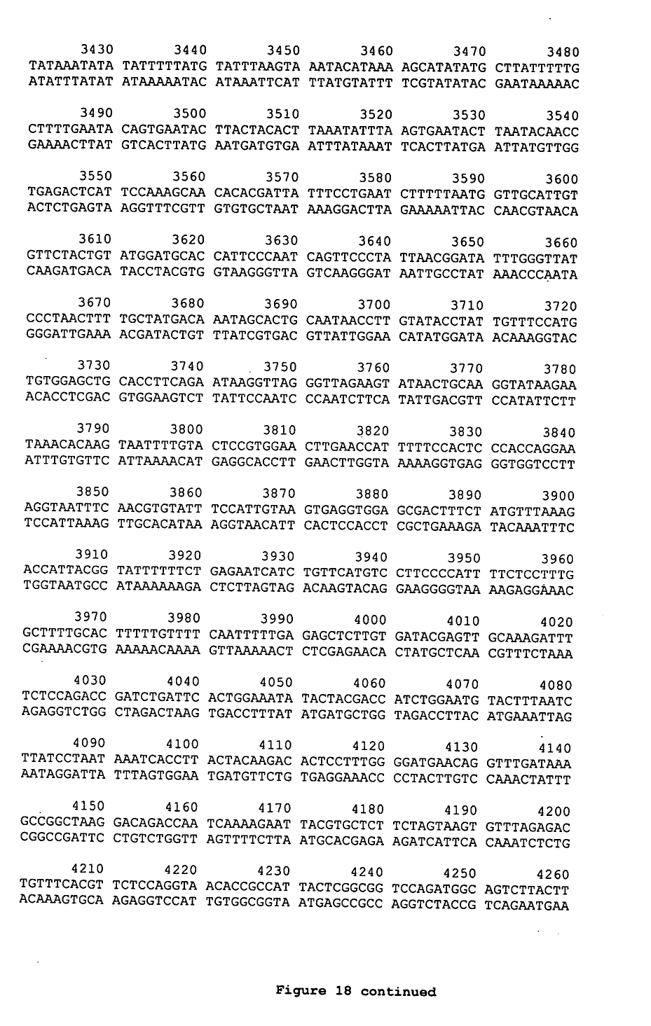

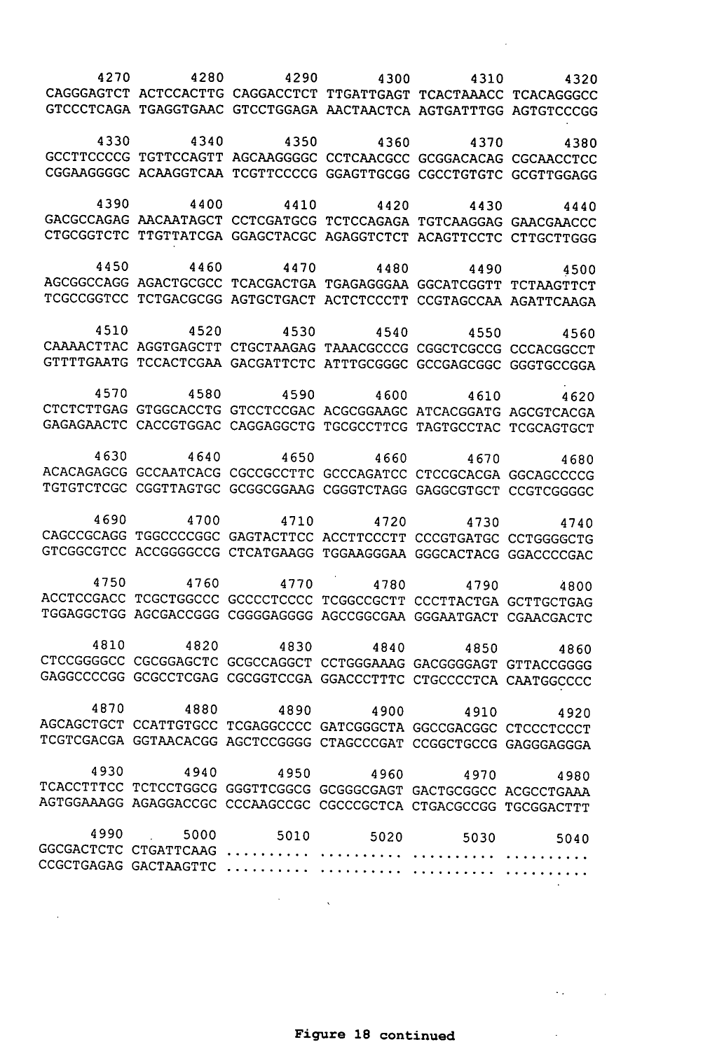

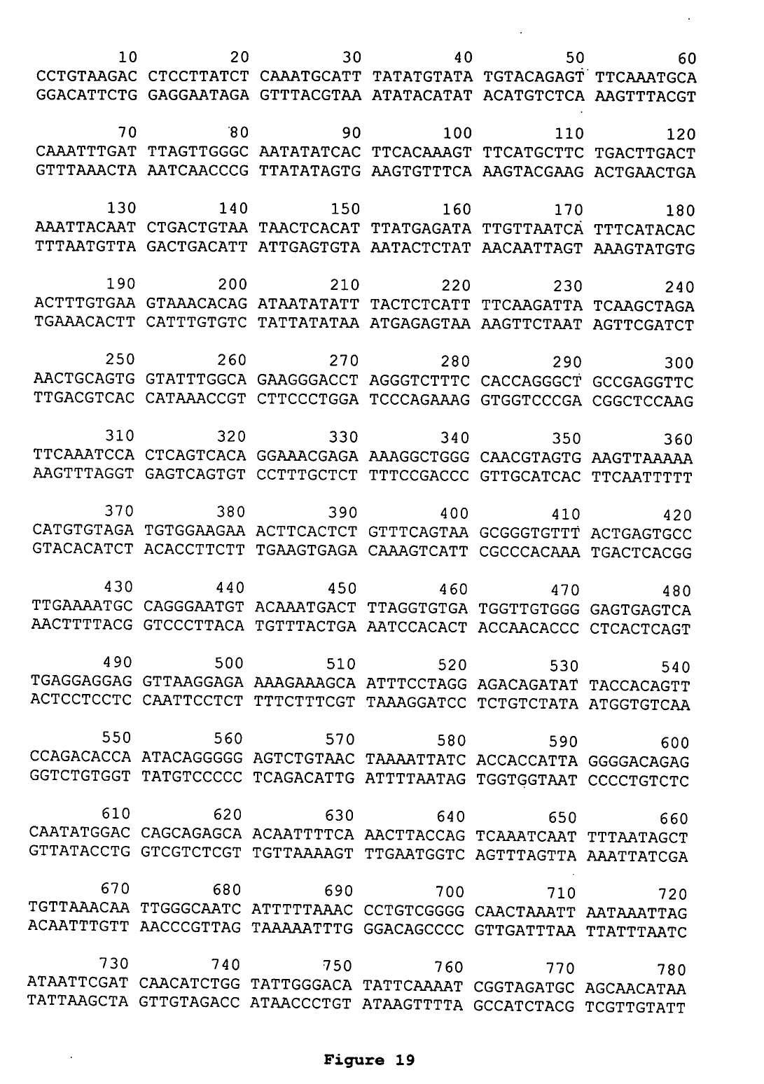

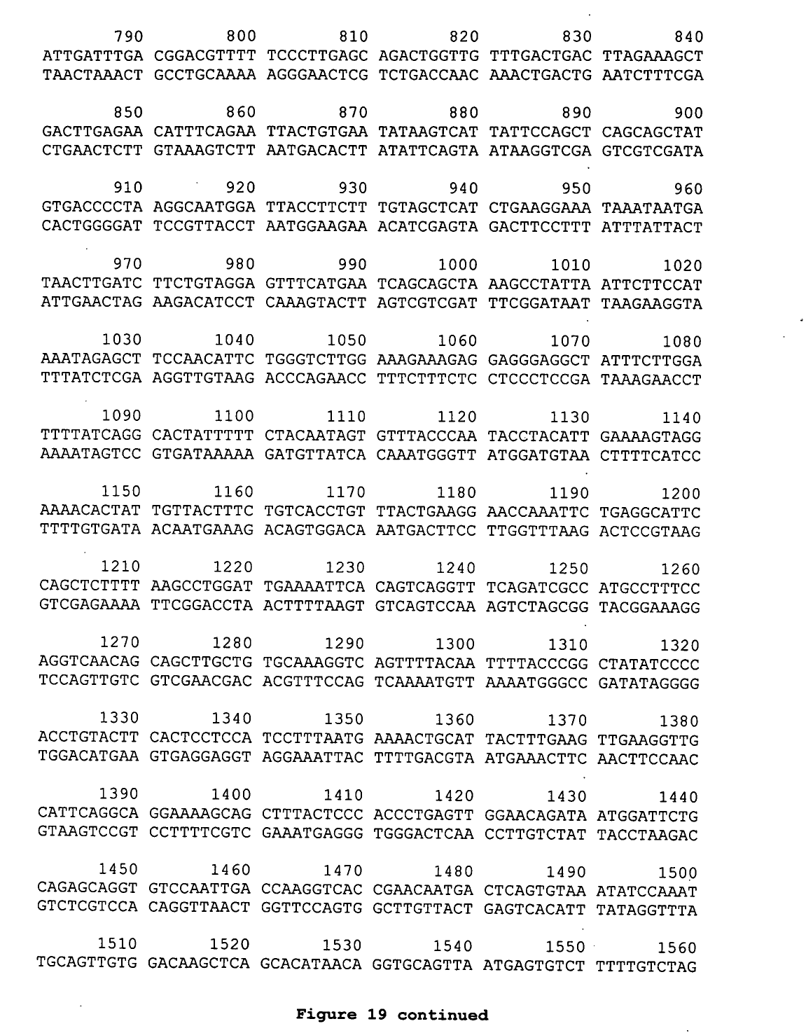

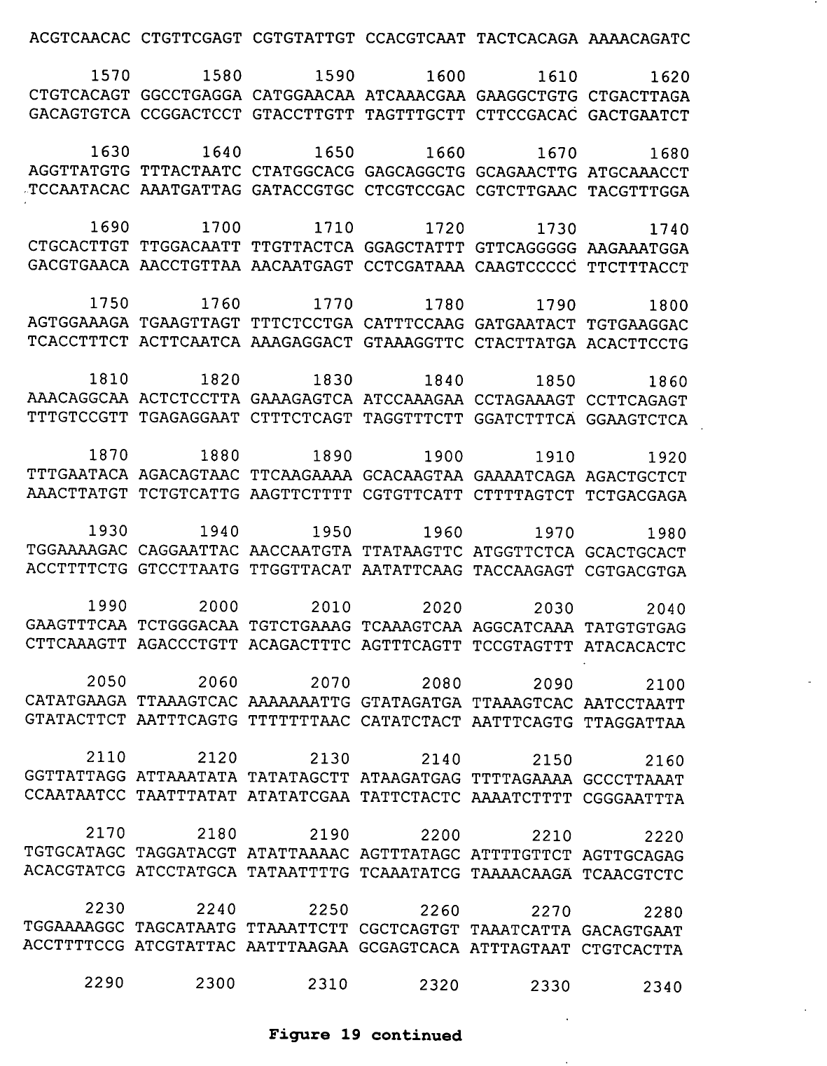

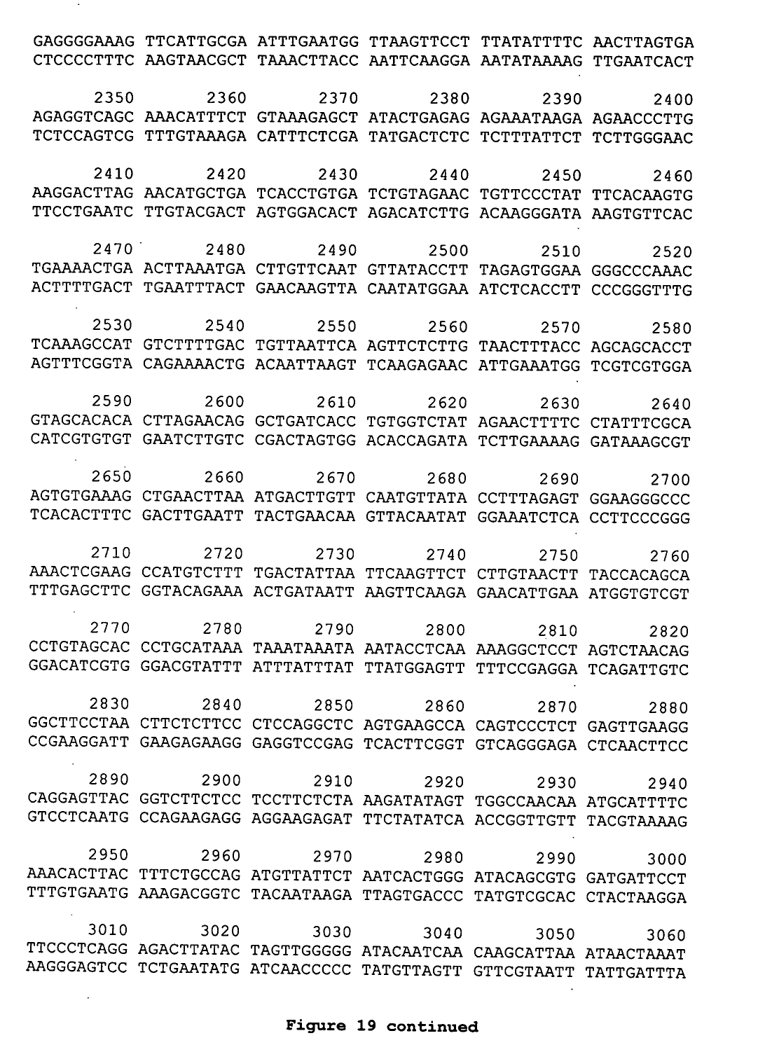

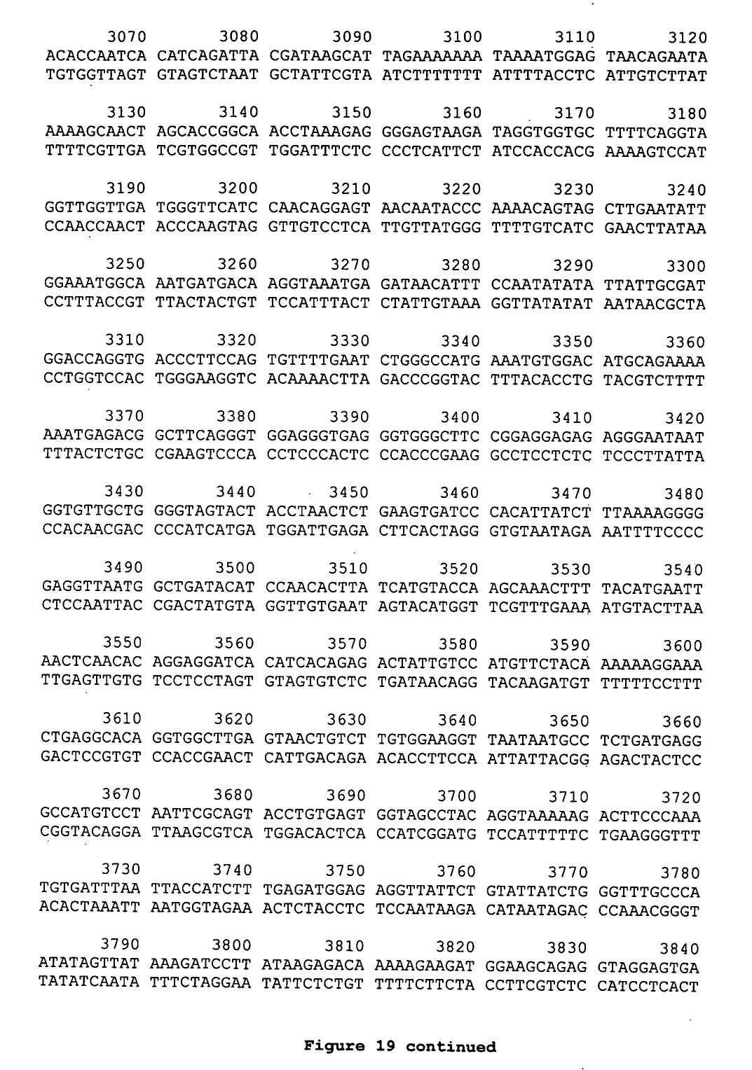

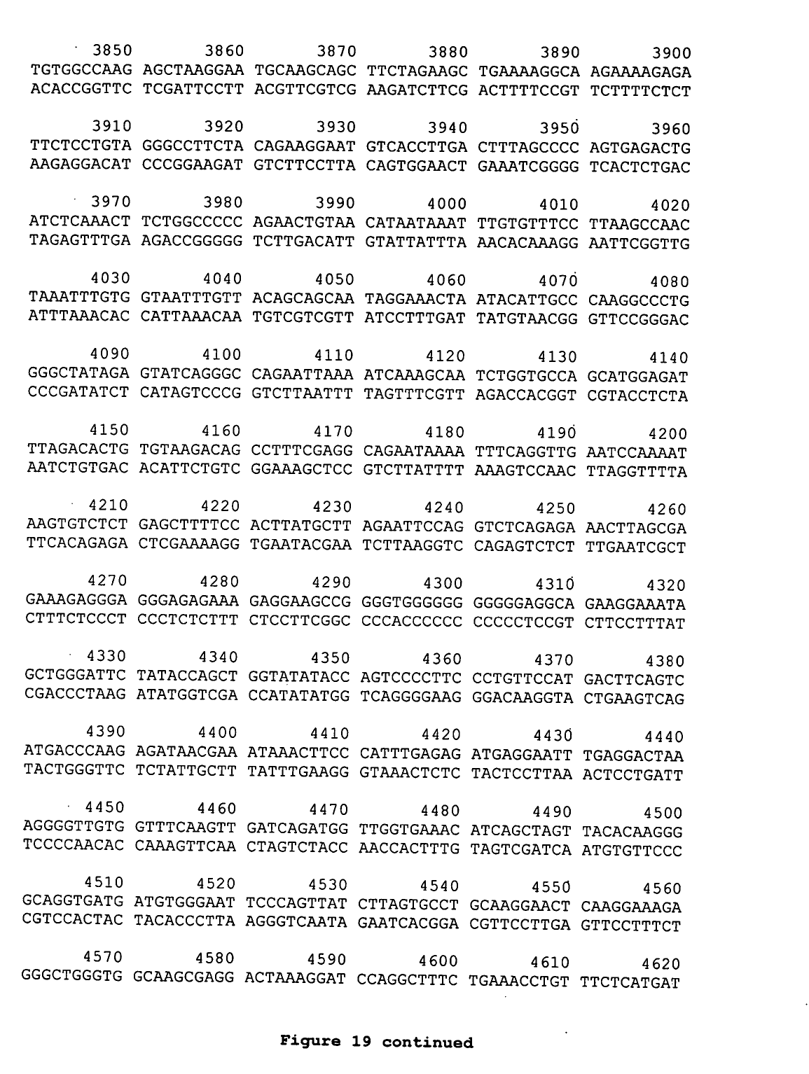

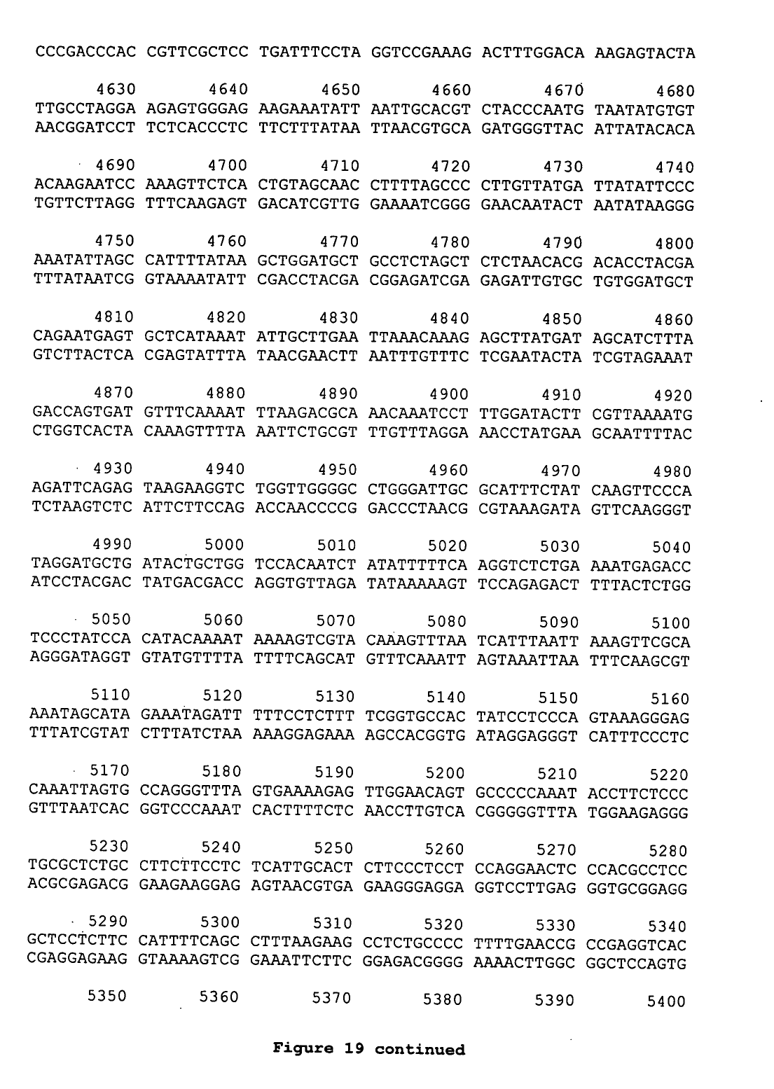

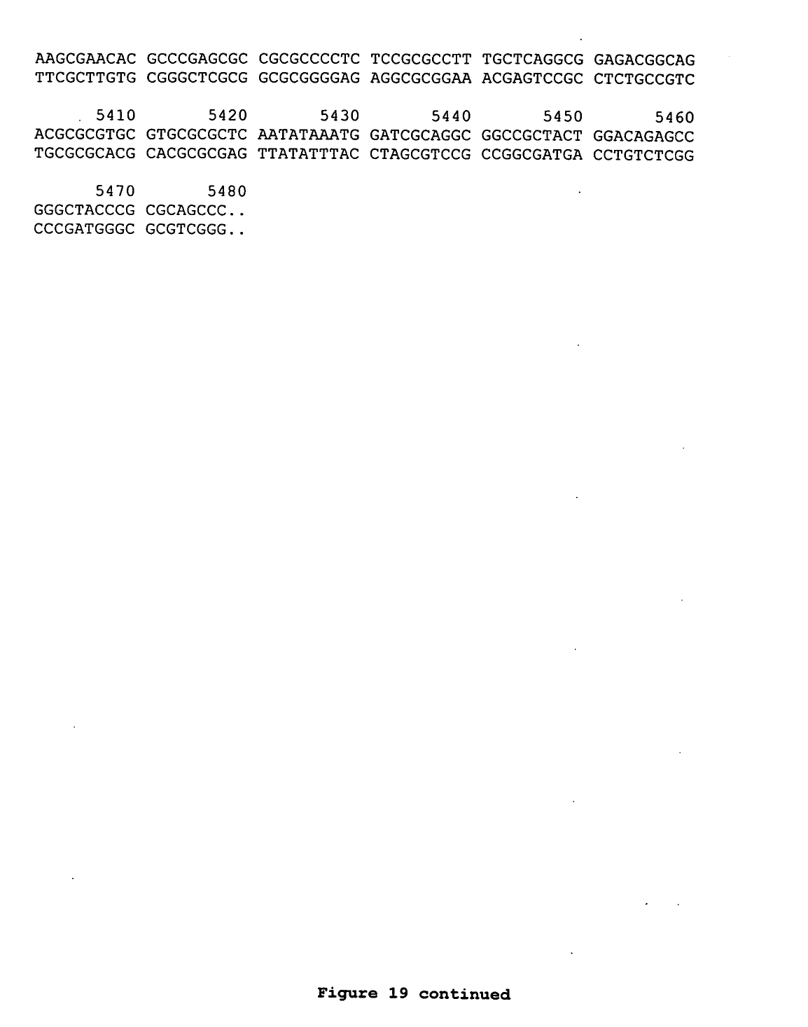

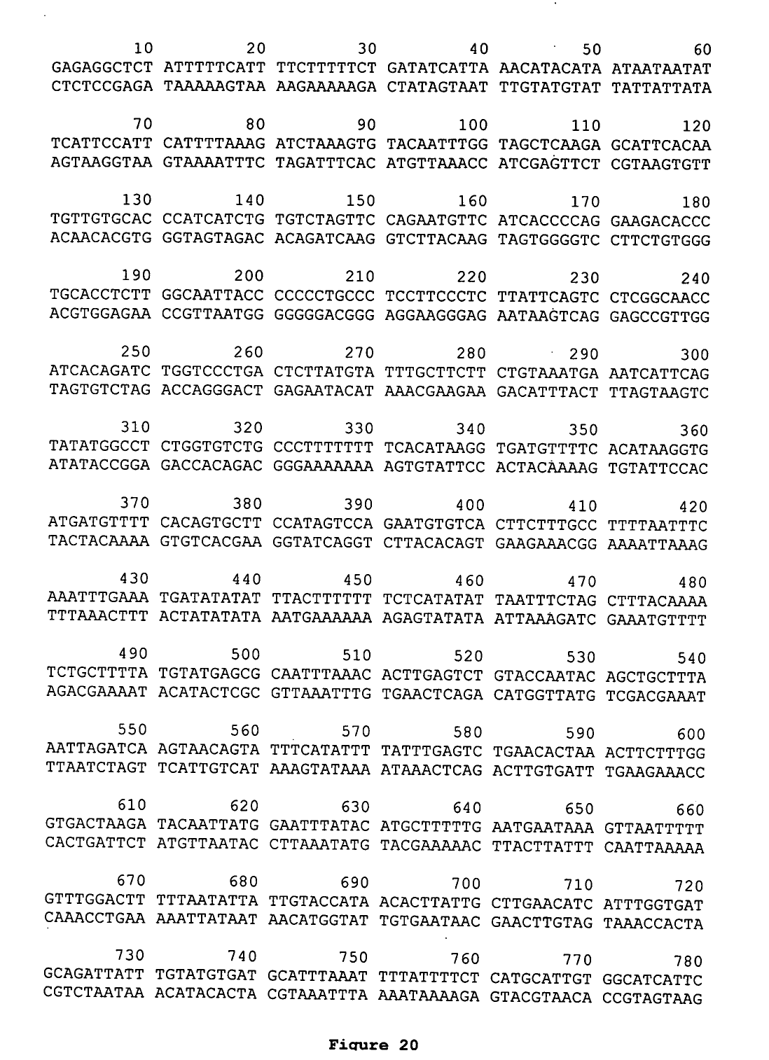

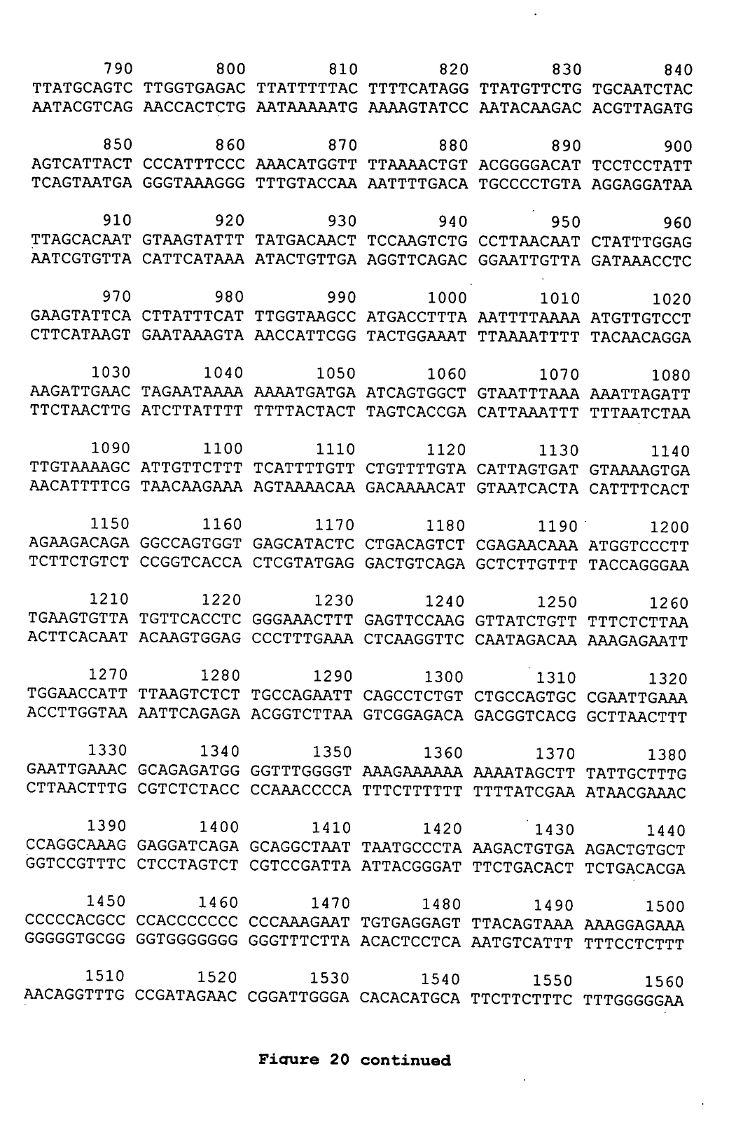

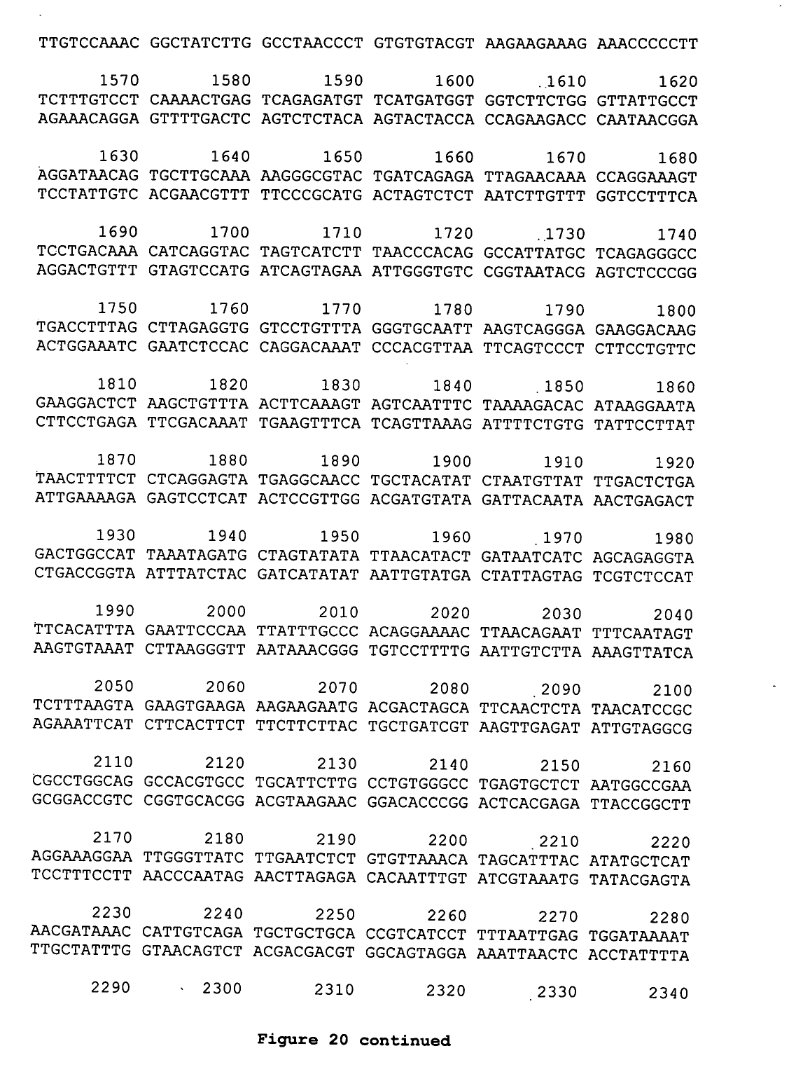

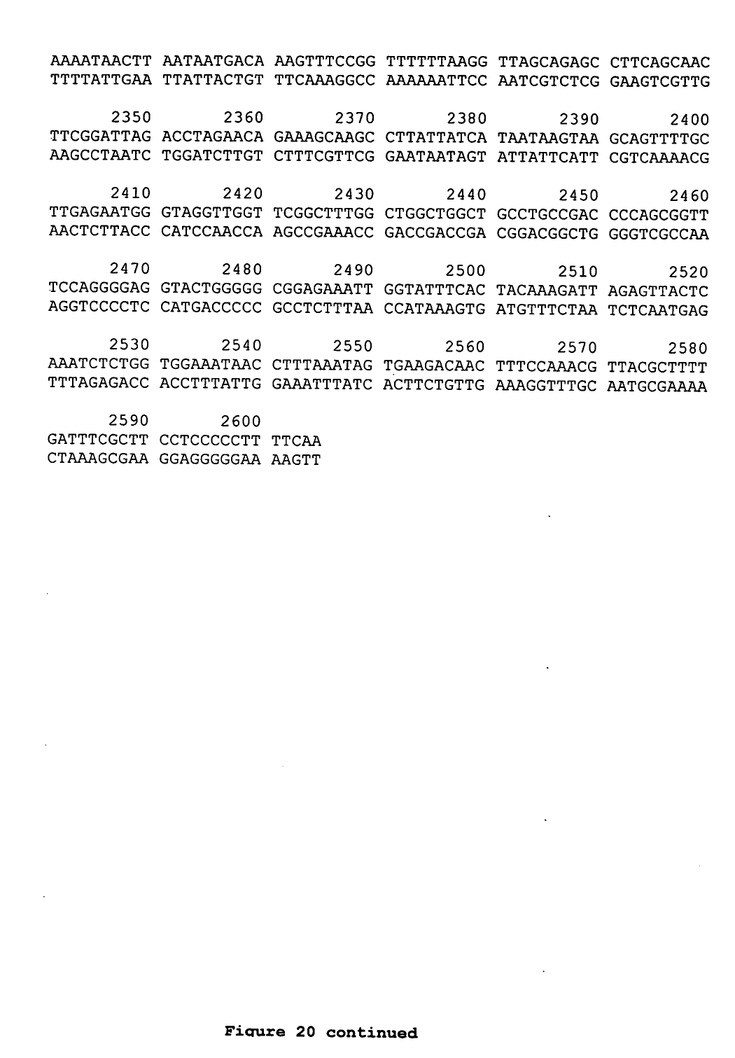

In a similar fashion, transgenes were created based on 5 kb of human LHX9 promoter sequence (FIG. 17 (SEQ ID NO: 2)) driving the coding sequence of yellow fluorescent protein (YFP), on human WT-1 promoter sequence (FIG. 18 (SEQ ID NO: 3)) driving the coding sequence of green fluorescent protein (GFP) and on pig DAX1 promoter sequence (FIG. 19 (SEQ ID NO: 4)) driving the coding sequence of yellow fluorescent protein (GFP). Also, transgenes were created based on 2.6 kb of pig SRY promoter sequence (FIG. 20 (SEQ ID NO: 5)) driving the coding sequence of yellow fluorescent protein (YFP). These transgenes were each microinjected into FVB/N embryos and depending on the transgene, from 2 to 6 independent lines of mice for each transgene were generated.

### EXAMPLE 2

Gata4 Promoter Transgene Expression in Mice Embryos

All GATA4p-GFP lines studied have revealed the same patterns of fluorescence, as described below.

Initially fluorescence was observed in neonatal animals of the F1 generation. From the exterior of the neonatal animal, transgenic animals were identified by retinal pigmentation when viewed with visible light, and when viewed using a fluorescence stereomicroscope equipped with filters for GFP, by lines of fluorescence on either side of the spinal column, two large points of fluorescence representing the olfactory bulbs of the nose, four lines of fluorescence within the tail, and punctate fluorescence within the skin.

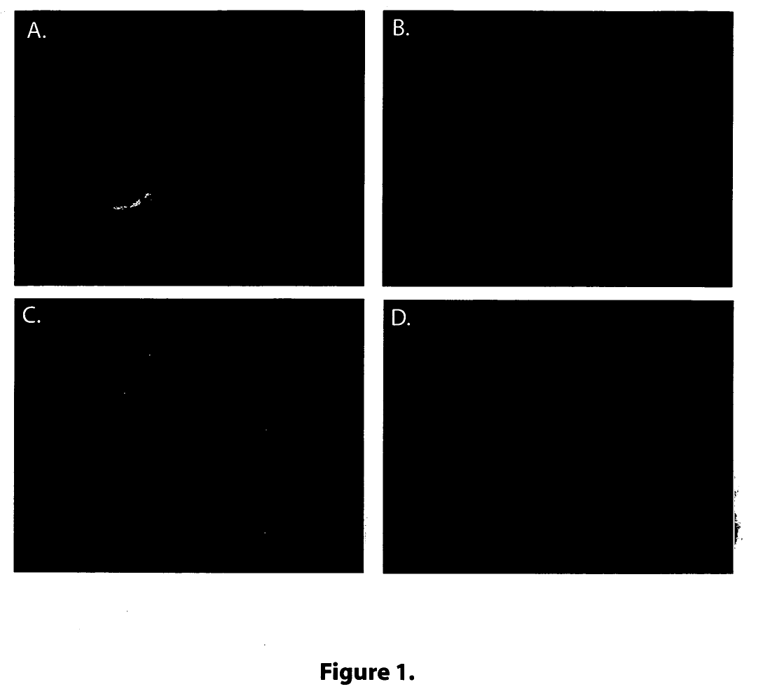

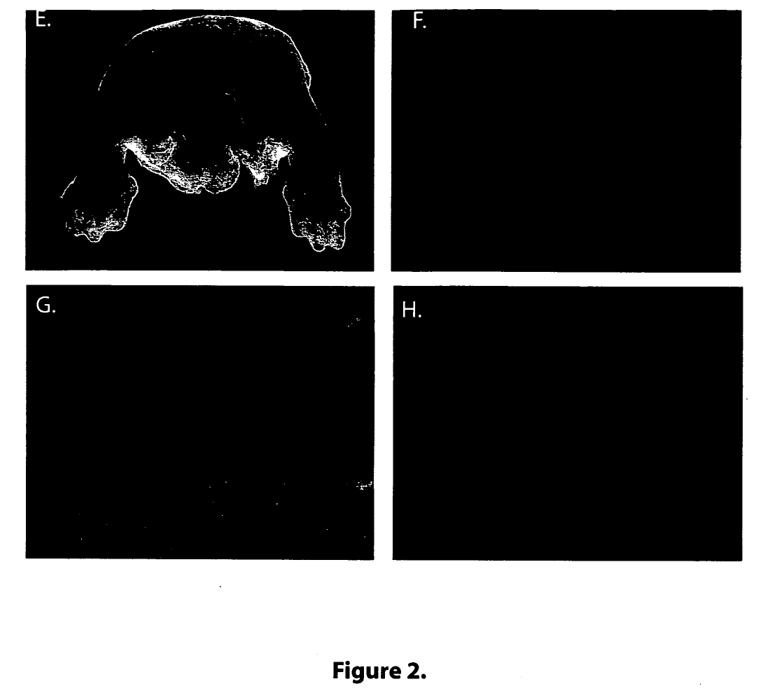

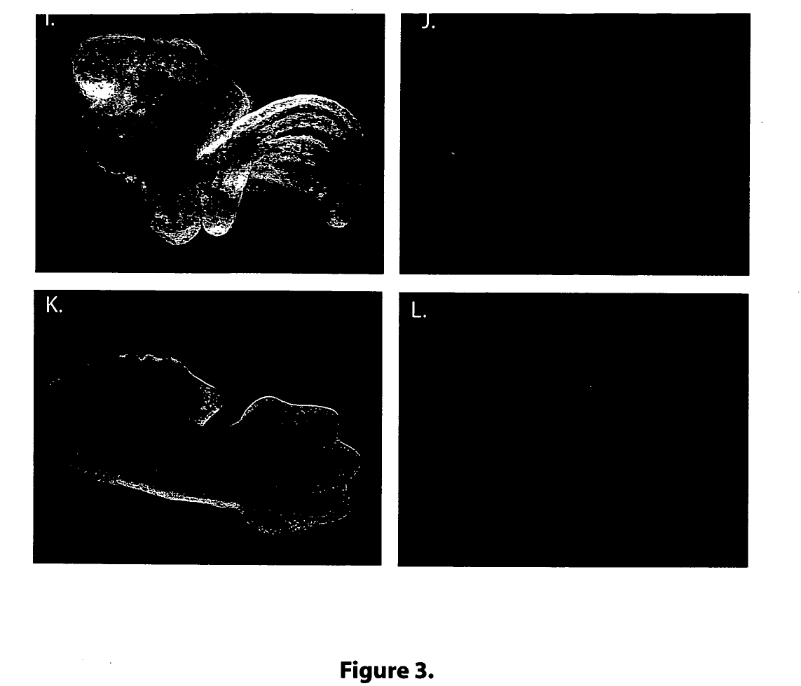

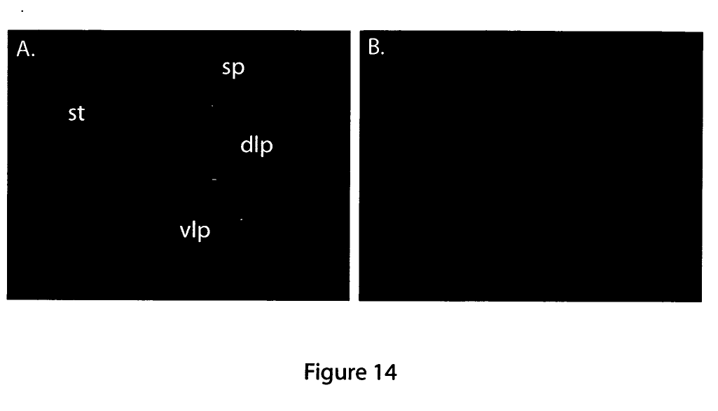

Dissections of embryos were performed to further characterize the expression patterns of fluorescence. The earliest embryonic day observed was e8.5, just before turning of the mouse embryo and when the neural pores are still open (data not shown). Fluorescence is associated with the region of the neural tube where fusion had already occurred, and on the lateral lips of the neural plate in the cranial region where the cranial neuropore was still open. By day e9.5, with gastrulation and neurulation complete and the embryo turned, fluorescent cells are readily evident within the rostral head and branchial arch regions (data not shown), as well as within the heart loop (data not shown). Tracts of fluorescent cells associated with the somites were evident. By day e10.5 these somite associated tracts are much more prominent, and it is evident that these tracts are segmented and elongated in a ventral pattern (FIG. 1, panel B). When the embryo is viewed from the dorsal aspect, this segmentation is evident, as is the fact that the neural tube does not display fluorescence (FIG. 1, panel D). Also at e10.5, fluorescent cells are evident within the primitive digestive tube (data not shown). By embryonic day e11.5, the stomach, esophagus and intestines are strongly fluorescent and will remain so for the duration of gestation (FIG. 3, panel J). By day e13.5, fluorescence is evident within the heart and strongly associated with the outflow tract and the descending aorta is fluorescent until the lumbar region (FIG. 2, panel F). The gastric trunk area in the region of the genital ridge is strongly fluorescent; this fluorescence will resolve itself within the next several days to mark the developing testes, the adrenal medulla, and the celiac ganglia. The segmented peripheral nerve tracts are demarcated by fluorescence (data not shown). The dorsal non segmental pattern of neural crest cell migration is marked by expression of fluorescence within the dermis of the trunk (data not shown). At day e13.5, the dorsal and ventral lobes of the pancreas, as well as a segment of the intestine also fluoresce (FIG. 14).

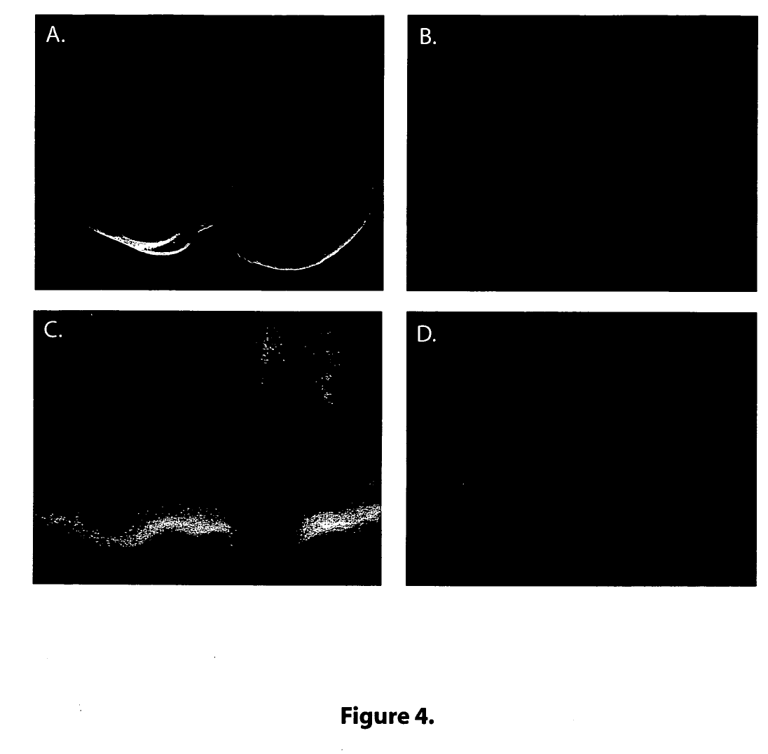

As expected, Gata4p-RFP transgenic mouse lines showed expression patterns (FIG. 4) similar to those described for the Gata4p-GFP lines with the obvious difference that Gata4p-RFP positive embryos have neural crest cells marked with red fluorescence rather than green fluorescence.

### EXAMPLE 3

Comparison of Fluorescence Expression Pattern in a Gata4-GFP Transgenic Mouse with Mouse Endogenic Gata4 mRNA Expression

In situ hybridization (ISH) of endogenous Gata4 mRNA was performed on e11.5 embryo and the staining pattern so obtained was compared with the fluorescence pattern obtained in a e11.5 embryo from a Gata4-GFP transgenic mouse line.

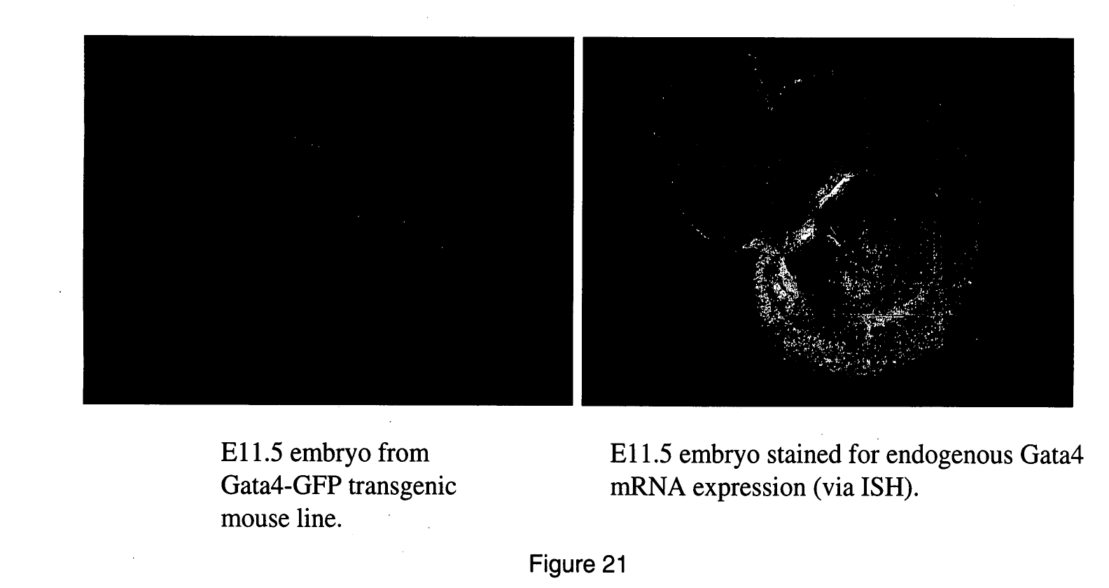

As is apparent from the similarity in the staining expression pattern in the mouse of the right panel of FIG. 21 with the fluorescence expression in the mouse of the left panel, the Gata4 gene is expressed in migrating neural crest cells and the Gata4-GFP transgenic mouse usefully reflects endogenous neural crest cell migration.

### EXAMPLE 4

LHX9 Promoter Transgene Expression in Mice Embryos

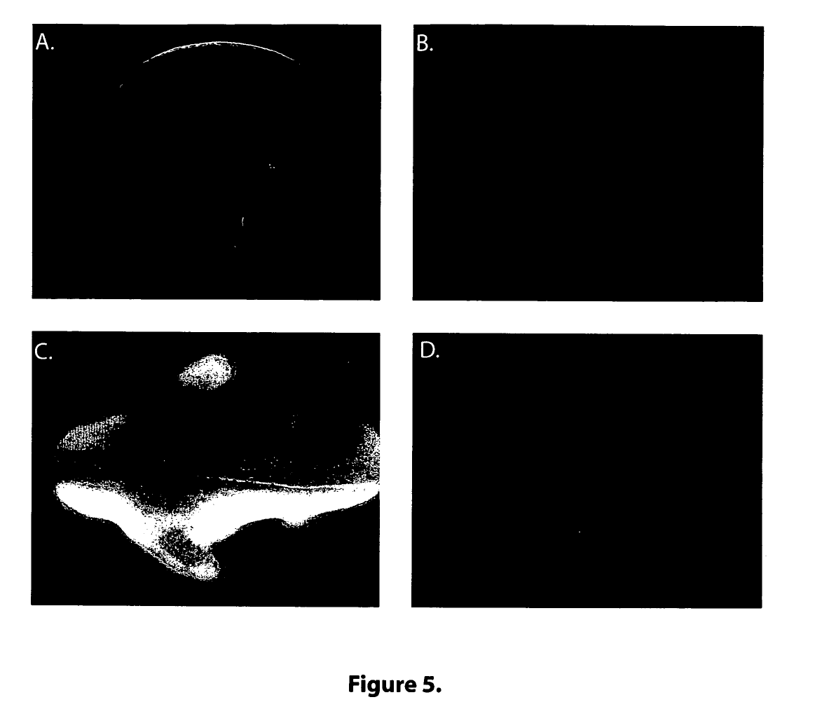

LHX9p-YFP transgenic mouse fluorescence expression patterns were observed to be quite similar to those of the Gata4 promoter transgenic mouse, in that migrating neural crest cell populations were marked (FIG. 5). This time, the marking was with yellow fluorescent protein as opposed to green or red fluorescent protein. It is to be noted that yellow fluorescence from YFP is actually green-yellow, with a substantial overlap with GFP. Minor differences were observed: fluorescent marking of cranial structures was somewhat more extensive while fluorescent marking in cardiac structures was less extensive than in the Gata4 promoter transgenic models. Troncal neural crest was equally well marked in the LHX9p-YFP and the Gata4p-GFP and RFP models.

### EXAMPLE 5

WT-1 Promoter Transgene Expression in Mice Embryos

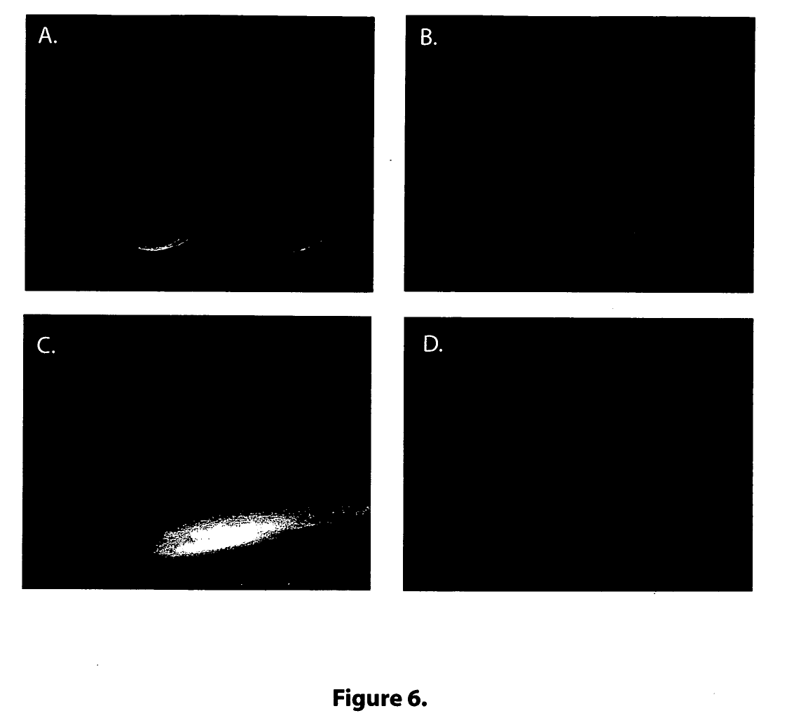

WT1p-GFP transgenic mouse fluorescence expression patterns were observed to be quite similar to those of the Gata4 and LHX9 promoter transgenic mice expression patterns, in that migrating neural crest cell populations were marked (FIG. 6). This time, the marking was with green fluorescent protein. The marking of structures within the cranial and troncal regions of the embryo appeared equivalent to that by the LHX9p-YFP transgene; the heart was not marked.

### EXAMPLE 6

GATA4 Promoter Transgene Expression in Newborn Mice

Dissection of neonatal animals revealed fluorescence in tissues indicative of a neural crest origin: the peripheral nervous system including the dorsal root ganglia, the autonomic ganglia, peripheral tissue nerve tracts; specific sites within the heart; the aorta including the arch and abdominal aorta; the gastrointestinal tract including the esophagus, stomach, small intestines, cecum, and large intestines; the adrenal medulla. In the neonatal brain, various specific tracts fluoresce, including tracts to the ears and to the nose. The meninges are fluorescent, as are the trigeminal nerve tracts. The spinal cord does not fluoresce.

To observe the presence of neural crest cells in neonatal transgenic mice, five day old (d5) Gata4p-RFP mice were sacrificed and skin samples were taken from the back, the head, and the whisker (upper lip) regions. Skin samples were placed onto glass cover slips, and the underside (hypodermal side) was observed at 80× magnification using a stereomicroscope (Leica™ MZ FLIII) with visible light and with the RFP filter set. Digital images were taken using a CCD camera (Nikon™ DMX1200) with appropriate image capture software (Nikon™ ACT-1). Alternatively, strips of skin were embedded into 3% agarose blocks and sliced in cross section using a vibratome (Leica™ VT-1000) to give 300 □M thick slices. These slices were mounted onto a microscope slide, and observed with the stereomicroscope or with an upright microscope (Nikon Eclipse™ E800) for taking images at 100× magnification with visible light and with a RFP filter set.

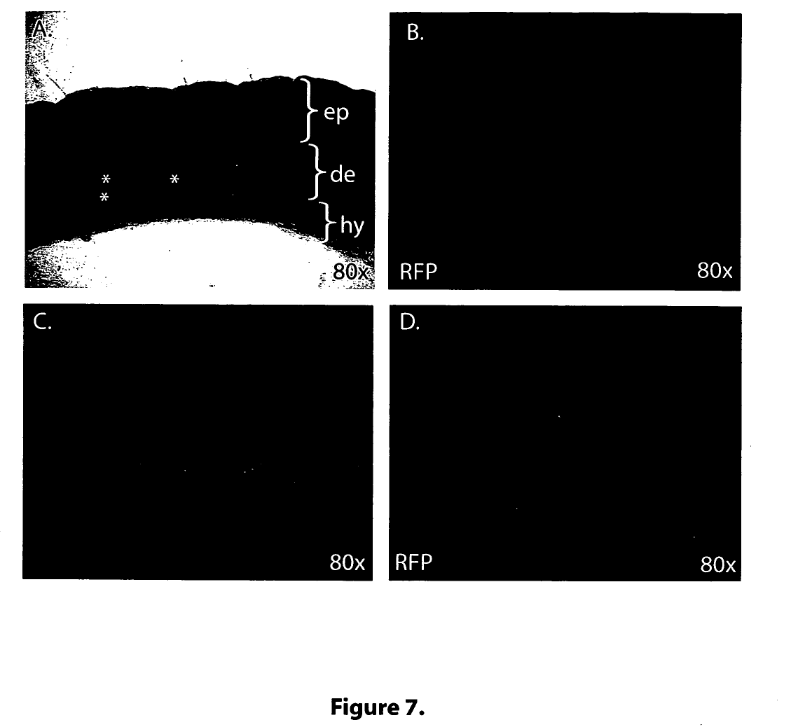

FIG. 7 presents a mouse newborn neural crest expression pattern in a skin sample of a 5 days old newborn transgenic mouse comprising the GATA4 promoter driving the RFP transgene. Left panels A (cross section of skin showing epidermis, dermis, hypodermis, hair follicules and dermal papillae, 80×) and C (bottom view of the skin sample hypodermal surface, 80×) shows the skin sample under visible light, while the right panels B and D present the skin sample views of panels A and C, respectively, with RFP filters to show neural crest cells location.

### EXAMPLE 7

LHX9 Promoter Transgene Expression in Newborn and Adult Mice

Dissection and sample preparation of the 5 days old newborn transgenic mouse comprising the LHX9 promoter driving the YFP transgene were performed as described in Example 6 above except that at 80X and 100× magnification, a YFP filter set was used. Additionally, the skin section was observed with confocal optics (Nikon D-Eclipse™ C-1) at 400× magnification, using bright-light and green-yellow channels, and images procured using the C-1 image capture program.

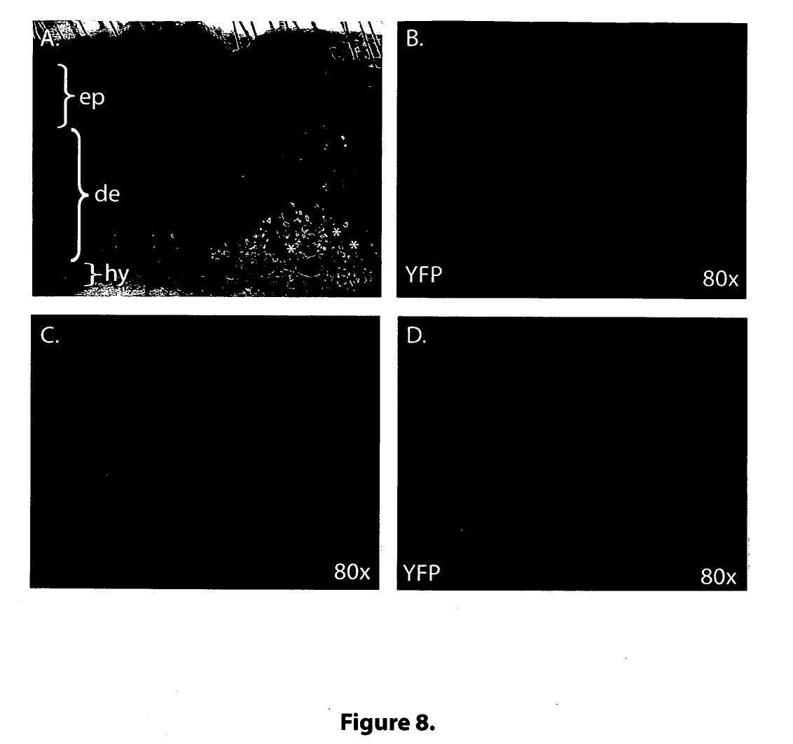

FIG. 8 presents the neural crest expression pattern in these skin samples. Left panels A (cross section of skin showing epidermis, dermis, hypodermis, hair follicules and dermal papillae, 80×) and C (bottom view of the skin sample hypodermal surface, 80×) of FIG. 8 show the skin sample under visible light, while the right panels B and D of FIG. 8 present the skin sample of panels A and C, respectively, with a YFP filter set to show neural crest cells location.

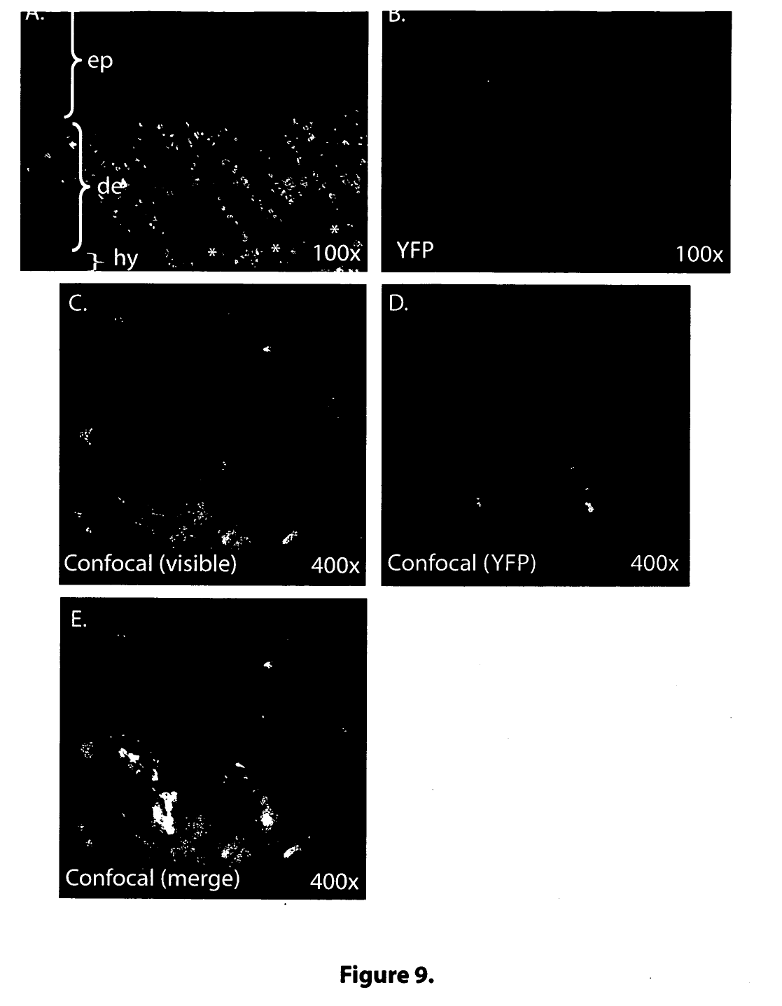

Skin samples from a 10-week-old (adult) transgenic mouse comprising the LHX9 promoter driving the YFP transgene were obtained as described in Example 6 above. FIG. 9 shows the neural crest expression pattern in those skin samples. Panels A (cross section of skin showing dermal papillae, 100×) and C (cross section of skin showing dermal papillae, 400×) show the skin sample under visible light and confocal optics, respectively, while panels B and D present the skin sample of panels A and C, respectively, with a YFP filter set to show neural crest derived cells location. Panel E presents a merged image of panels C and D.

By day 17 after birth, fluorescence is still readily evident but in general is less dramatic than in embryonic and neonatal tissues, due at least in part to the thickness and opacity of tissues. In the adult animal (eight weeks after birth), fluorescence was still readily evident in tissues including peripheral nerves, ganglia, intestinal tract and skin.

### EXAMPLE 8

WT-1 Promoter Transgene Expression in Newborn Mice

Dissection and sample preparation of the 5 days old newborn transgenic mouse comprising the WT-1 promoter driving the GFP transgene were performed as described in Example 6. A GFP filter set was used.

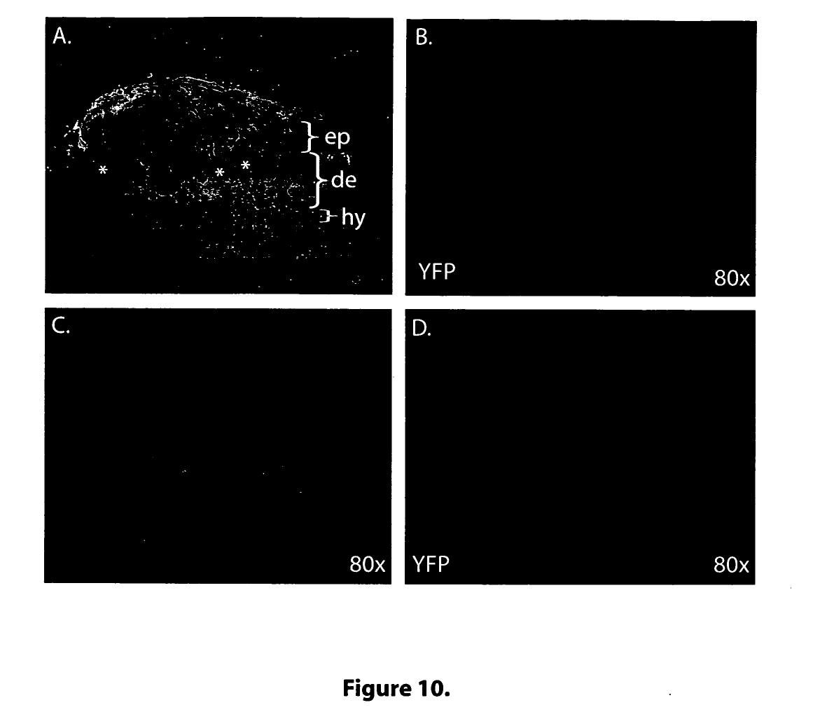

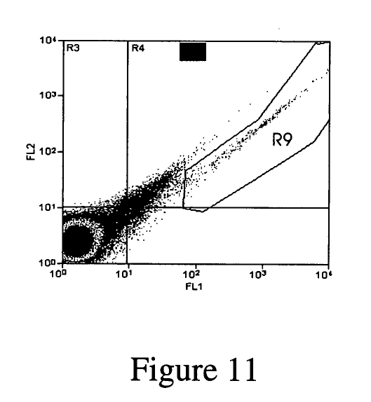

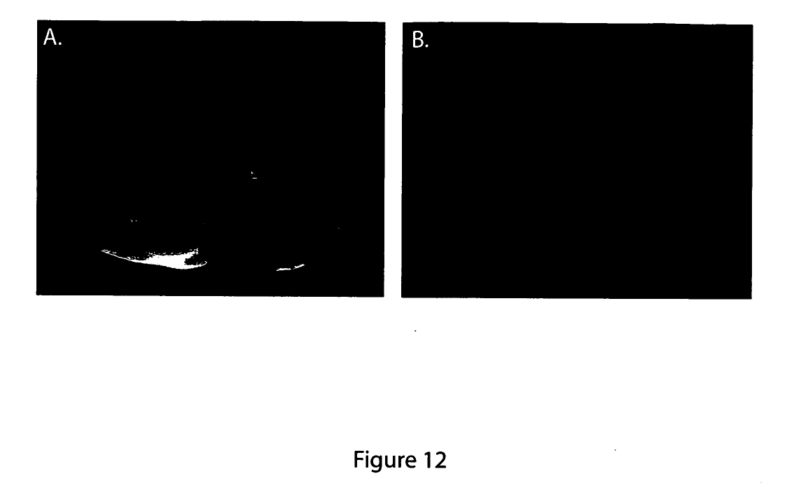

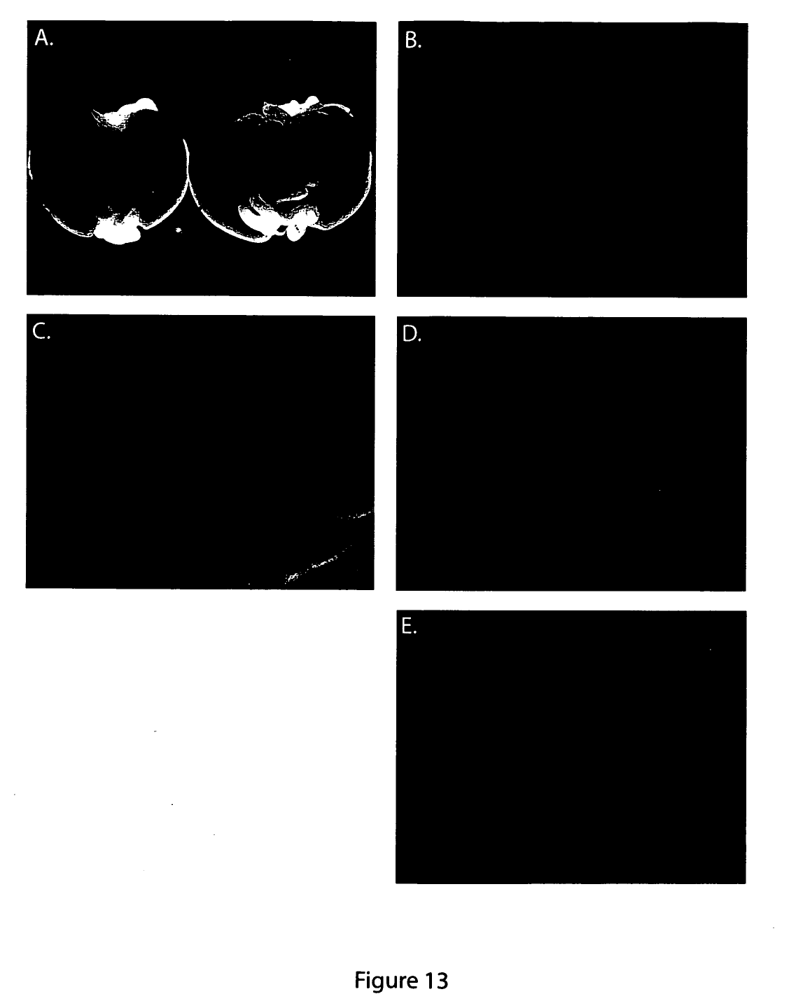

FIG. 10 presents the neural crest expression pattern in these skin samples. Left panels A (cross section of skin showing hair shaft 80×) and C (bottom view of the skin sample hypodermal surface, 80×) show the skin sample under visible light, while the right panels B and D present the skin sample of panels A and C, respectively, with a GFP filter set to show neural crest cells location.

### EXAMPLE 9

Isolation of Neural Crest Cells from Transgenic Mice Embryos

To isolate neural crest cells from mouse embryos (GATA4p-RFP, GATA4p-GFP, LHX9p-YFP and WT1p-GFP mouse lines), embryos were taken from timed gestations at embryonic day 8.5 (e8.5), e9.5, e10.5, e11.5, e12.5, etc. Embryos were then dissected to provide the appropriate tissue samples. Tissue samples were digested for 30 min at 37° C. in M2™ media (Sigma, St Louis Mo.) supplemented with collagenase Type III (50 U/ml, GibcoBRL) and dispase (2.4 U/ml, GibcoBRL). The resulting cell suspension was then subjected to fluorescence activated cell sorting (FACS) using a MOFLO™ (Cytomation) apparatus equipped with an EGFP, EYFP or a RFP filter set depending of the transgene observed.

### EXAMPLE 10

Isolation of Pancreatic cells from Transgenic Mice Embryos

To isolate pancreatic cells from mouse embryos (GATA4p-RFP, DAX1p-YFP, and SRYp-YFP mouse lines), embryos are taken from timed gestations at embryonic days e10.5, e11.5, e13.5, e15.5, etc. Embryos are then dissected to provide the appropriate tissue samples. Tissue samples are digested for 30 min at 37° C. in M2™ media (Sigma, St Louis Mo.) supplemented with collagenase Type III (50 U/ml, GibcoBRL) and dispase (2.4 U/ml, GibcoBRL). The resulting cell suspension is then subjected to fluorescence activated cell sorting (FACS) using a MOFLO™ (Cytomation) apparatus equipped with an EYFP or a RFP filter set depending on the transgene observed.

### EXAMPLE 11

DAX1 Promoter Transgene Expression

DAXp-YFP transgenic mouse fluorescence expression patterns were observed to be quite similar to those of the GATA4, LHX9 and WT1 promoter transgenic mice, in that migrating neural crest cell populations were marked (FIG. 14). This time, the marking was with yellow fluorescent protein. In addition, pancreatic progenitor cells were marked by at least day e11.5. Fluorescence was not maintained in neural crest derived tissues in newborn and adult animals as it is for the GATA4, LHX9 and WT1 promoter transgenic mice.

### EXAMPLE 12

SRY Promoter Transgene Expression

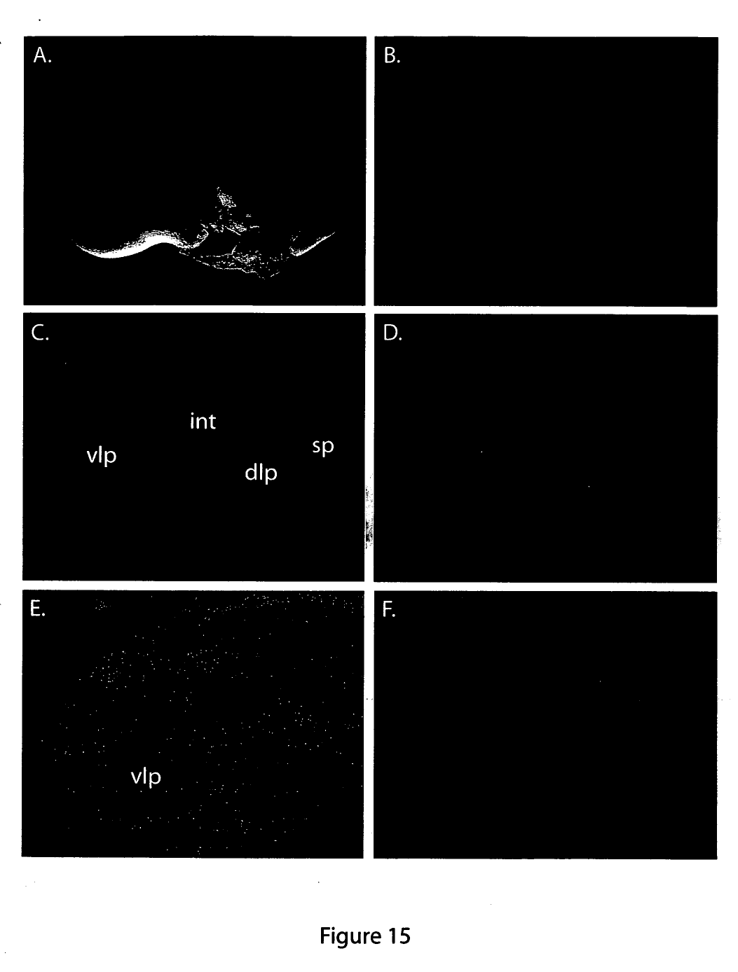

SRYp-YFP transgenic mouse fluorescence expression patterns were observed to be quite similar to those of the GATA4, LHX9 and WT1 promoter transgenic mice, in that migrating neural crest cell populations were marked (FIG. 15). This time, the marking was with yellow fluorescent protein. In addition, pancreatic progenitor cells, and genital ridge were marked by at least e13.5.

### EXAMPLE 13

GATA4 Promoter Transgene Expression in Pancreatic Tissues

The GATA4 promoter transgenic lines displayed readily visible fluorescence expression within the developing pancreas by at least e13.5 of gestation similar to that observed in the DAX1 and SRY promoter transgenic mouse lines.

### EXAMPLE 14

Isolation of Neural Crest Cells in Neonatal Mice

Strips of skin from five to nine day old (d5-d9) LHX9p-YFP transgenic mice were embedded into 3% agar blocks and sliced in cross section using a vibratome (Leica™ VT-1000) to obtain 300 □M thick slices, as described above. The dermal layer (as seen in FIGS. 7, 8, 9 and 10) was dissected from the epidermal and hypodermal layers of these skin slices with the aid of a scalpel blade and a dissecting microscope (Leica™ MZ FLIII), such that the only fluorescent cells within the dissected sample represented follicular papillae cells. Fluorescence was the result of presence of YFP protein due to expression of the LHX9p-YFP transgene. Dermal layers were pooled and cells disaggregated (37° C., 1 hour incubation) in an aqueous solution containing M2™ media (Sigma), dispase (2.4 IU/ml; GibcoBRL), and collagenase type III (50 IU/ml; GibcoBRL). The resulting cell suspension was then subjected to fluorescence activated cell sorting (FACS) using a MOFLO™ (Cytomation) apparatus and sorting on the presence of yellow fluorescence. A typical FACS cell profile is presented in FIG. 1, with FL1 axis representing intensity of yellow fluorescence. Cells from region 9, containing flourescence levels that were 100 to 1000 times those of the background levels, represent the fluorescently marked follicular papillae cells that were physically seperated from the unmarked dermal cell population. Table 2 below represents a tabulation of the FACS sorting events, where it is shown that approximately 0.3% of dermal cells consisted of fluorescently marked cells representing follicular papillae cells and presumptive neural crest stem cells.

TABLE 2
Fluorescence activated cell sorting results

| PRE-SORT YFP | | |
|---|---|---|
| Region | Count | % |
| Total | 105897 | 100 |
| R3 | 475 | 0.45 |
| R4 | 3535 | 3.34 |
| R5 | 100616 | 95.01 |
| R6 | 1271 | 1.2 |
| R9 | 330 | 0.31 |

sorted = 7000

### EXAMPLE 15

Neural Crest Cell Recovery

Experiments performed with different stages of Gata4pGFP embryos showed that the best recovery for cranial neural crest cells (NCS) is obtained using e9.5 embryos (i.e. about 7000 cells per embryo) while the best recovery for troncal NCS is obtained using e10.5 embryos (i.e. about 2000 cells per embroy). Furthermore, about 50 embryos (i.e. about 10 pregnancies) provided a useful amount of tissue for culture experiments.

### EXAMPLE 16

Expansion and Differentiation of Neural Crest Stem Cells

The Gata4p-GFP, Gata4pRFP, LHX9p-YFP, WT1-GFP, DAXpYFP and SRYpYFP mouse lines are used as the basis to isolate and manipulate pluripotential neural crest stem cells. Isolation and purification of early migrating neural crest cells is performed by embryo dissection, tissue digestion, followed by FACS isolation of fluorescent cells as described in Examples 9 and 14 above. The fluorescent cells are then plated onto Petri dishes at clonal (reduced) concentrations, and maintained in tissue culture under conditions to encourage cell proliferation while at the same time discourage cell differentiation. Clonal populations of early migrating neural crest cells represent neural crest stem cells. The differentiation potential of these clonal populations is then tested for their capacity to differentiate into different cell types, including neurons, glial cells, melanocytes, neuroendocrine cells, muscle cells, cartilage cells, and support cells. These neural crest stem cell populations and their differentiated cell types are useful for standardized pharmacological high throughput screening applications.

### EXAMPLE 17

Pluripotentiality of Neural Crest Cells Shown Via Tissue Culture

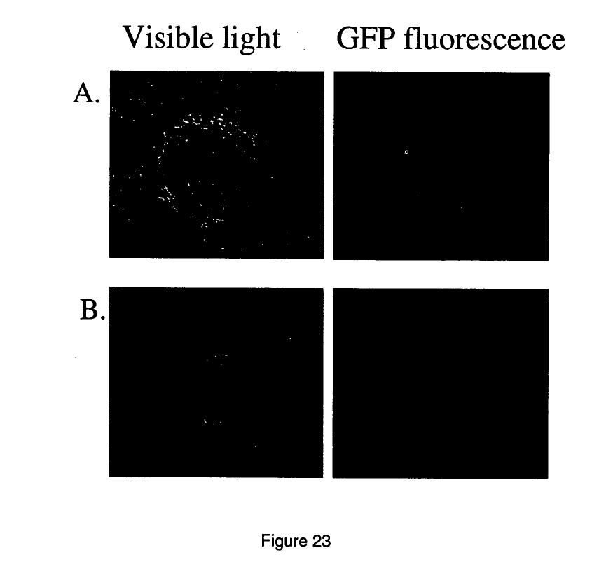

Cranial neural crest cells from about 50 e9.5 embryos were grown for 1 week under conditions that favour stem cells proliferation and disfavour cell differentiation. Growth with NeuroCult™ media (Stem Cell Technologies) plus Proliferation Supplement (Stem Cell Technologies; recommended concentrations), EGF (20 ng/ml), bFGF (40 ng/ml) and without fibronectin resulted in growth of neural crest spheres that were similar to neuro-spheres and which likely constitute neural crest stem cells. Panels A and B of FIG. 23 represent two examples of neural crest cells grown as spheres. Spheres can maintain or loose fluorescence over time.

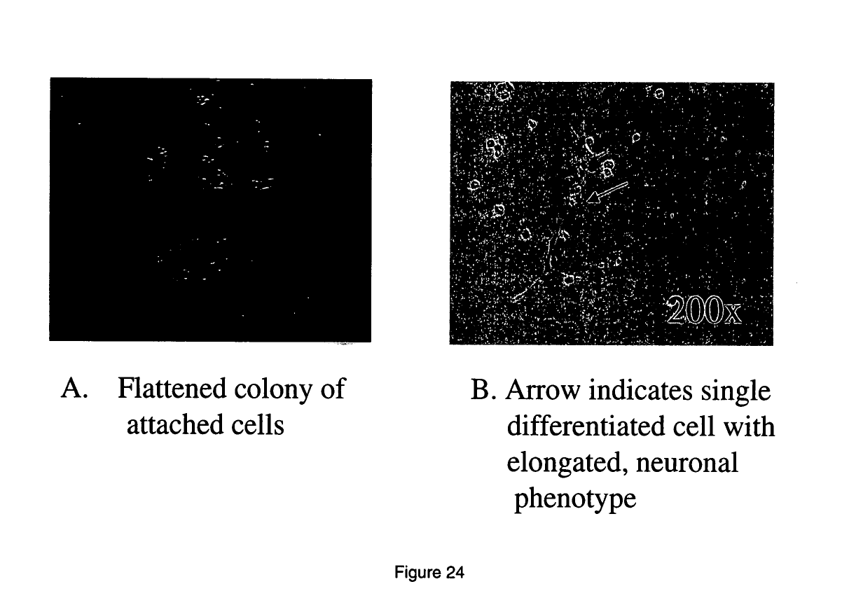

Troncal neural crest cells from about 50 e9.5 embryos were also grown for 1 week under conditions that favour cell differentiation and disfavour stem cells growth. The growth conditions were the standard culture conditions for mammalian cells: 37 degrees centigrade, 5% CO₂, NeuroCult™ media (Stem Cell Technologies) plus 5% Fetal Bovine Serum (FBS) and fibronectin. Growth on fibronectin resulted in adherence (panel A of FIG. 24) and morphological cell differentiation (panel B of FIG. 24) of the neural crest cells into flattened colonies or single differentiated cells with elongated, neuronal phenotype.

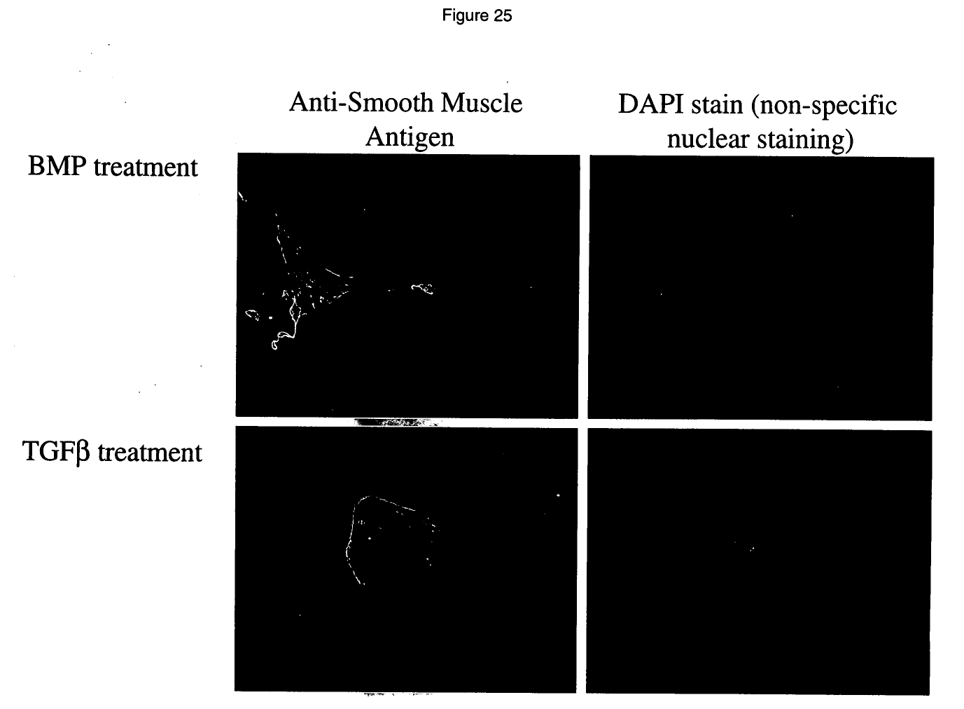

A new sample of troncal neural crest cells from about 50 e9.5 embryos was then grown on fibronectin for 2 weeks in the presence of differentiation factors including Bone Morphogenic Factor (BMF) (upper left panel of FIG. 25) or Transforming Growth Factor β (TGFβ) (lower left panel of FIG. 25). The growth conditions were the standard culture conditions for mammalian cells: 37 degrees centigrade, 5% CO₂, NeuroCult™ media (Stem Cell Technologies) plus 5% Fetal Bovine Serum (FBS). lrmunohistological staining with antibodies against smooth muscle antigen, a marker for smooth muscle cells shows in the left panels of FIG. 25 the presence of cells differentiated into such cells.

The fact that embryonic neural crest cells, when allowed to attach to tissue culture surfaces, will grow as flat colonies and will differentiate into different cell phenotypes shows the stem cell potential of neural crest cells.

### EXAMPLE 18

Generation of Promoter Sequences from Other Animal Species

Useful promoter sequences for the present invention can be derived from additional animal species via standard molecular cloning procedures. Hence, any animal promoter for the GATA4, LHX9, WT-1, DAX1, SRY or for any other gene that is expressed specifically in neural crest cells and in pancreatic tissues can be used in accordance with the present invention.

If the genome of the animal species from which the promoter is to be derived is publicly available (i.e. public sequence databases of plasmids, lamda or BAC clones), the promoter sequence can be obtained directly from it by isolating a useful sized sequence upstream of these genes. 5 Kb nucleic acid sequences were used as promoter sequences in Examples presented herein. The promoter sequence can then be physically generated via standard PCR amplification of genomic DNA for the animal in question.

If the whole genome of the animal species from which the promoter is to be derived is not publicly available but the coding sequence for the gene such as GATA4, LHX9, WT-1, DAX1 or SRY is, then the coding sequence can be used to physically generate the promoter sequences in question. This time, one sided or anchored PCR is performed on genomic DNA of the animal species in question to generate the desired promoter sequences.

If neither the genome nor the coding sequence of the gene in question is publicly available, then heterologous DNA primers based on known sequences for the gene of interest can be designed to amplify the coding regions of the unknown species. Then, the promoter sequence is obtained as described above.

Although a nucleic acid sequence of 5 Kb is generally an appropriate size for the promoters of the present invention, a person of ordinary skill in the art will understand that the size may be generally varied without affecting the expression. A person of ordinary skill in the art will also understand that for certain promoters, a different size is preferably used to obtain the appropriate expression. Hence, although the promoter of 2.6 Kb and an other one of 1.6 (data not shown) used for the SRY transgene produced the neural crest expression, a longer promoter (4.6 Kb) did not. It is thus believed that the longer promoter contained a sequence that inhibited neural and pancreatic expression.

### EXAMPLE 19

Screening Assays

Neural crest cells (i.e. including neural crest stem cells and differentiated neural crest cells) and pancreatic progenitor cells isolated through the method of the present invention provide an unlimited source of purified primary cells in tissue culture for cell based functional assays including high throughput assays. These assays could involve target molecule identification and validation, the tracking of cellular events, and the screening of compounds for pharmacological efficacy or toxicity. The response of neural crest cells derivatives such as Schwann cells, peripheral nerve cells, enteric nerve cells, neuroendorine cells, otic sensory cells, and melanocytes to candidate pharmacological agents can then be tested. The response of pancreatic cells such as pancreatic progenitor cells, endocrine cells (such as A, B, D and pp cells), exocrine cells and ductal cells to pharmacological agents can then be tested.

### EXAMPLE 20

Gene Expression Phenotyping of Fluorescent Neural Crest Cells

The gene expression phenotyping of fluorescent neural crest cells was performed via RT-PCR on FACS sorted fluorescent cells from e9.0 to e9.5 Gata4p-GFP embryos.

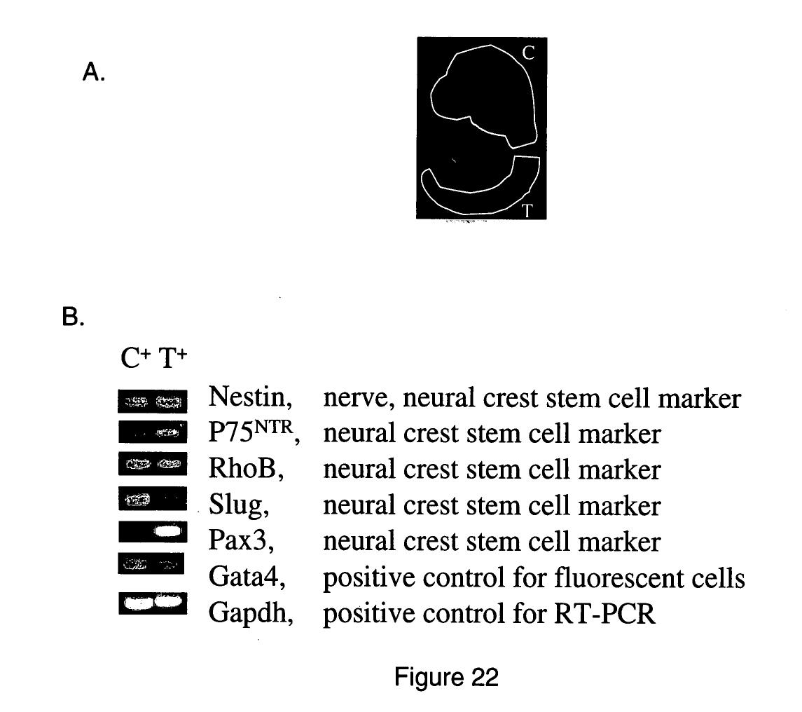

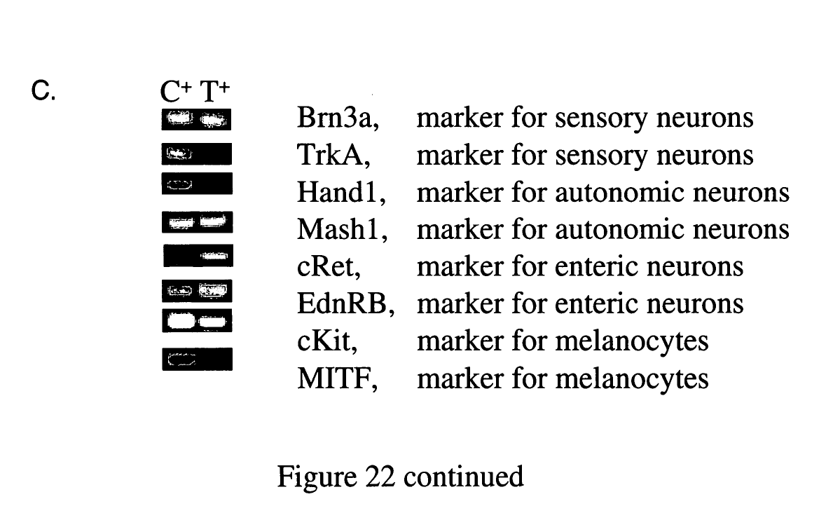

Dissections of e9.5 Gata4p-GFP embryos was performed to derive cranial (C) and troncal (T) tissues (See upper panel of FIG. 22). These tissues were then digested with trypsin and the resulting cell suspensions separated by fluorescent activated cell separation into fluorescent (i.e. neural crest) and non fluorescent cell populations. RT-PCR expression analysis was then performed on these populations.

RT-PCR expression analysis for neural crest stem cell markers was then performed using Gata4 as a positive control for fluorescent cells, and Gapdh as a positive control for RT-PCR. It confirmed the presence of a number of neural crest stem cell markers in these cells, namely Nestin; P75NTR; RhoB; Slug; and Pax3. It also confirmed the presence of neural crest restricted fate markers in the cells, namely cells that are more differentiated (i.e. having a smaller differentiated repertoire) than stem cells but that are still not fully differentiated, namely markers for sensory neurons i.e. Brn3a and TrkA; markers for autonomic neurons i.e. Hand1 and Mash1; markers for enteric neurons i.e. cRet and EdnRB; and markers for melanocytes i.e. cKit and MITF.

The presence of neural crest stem cells markers in the fluorescently marked neural crest cells confirm that these fluorescently marked neural crest cells comprise a neural crest stem cell population.

### EXAMPLE 21

Gene Profiling Assays

Neural crest cells and pancreatic cells isolated in accordance to the present invention can be more systematically characterized. For instance, their total RNA can be isolated and assayed in gene chips such as those from Affymetrix™. The proteins expressed on the surface of these cells can then be identified and antibodies specific to these proteins can be produced. These antibodies can then be used to identify human neural crest cells orthologs in adult human hair follicles for instance.

Although the present invention has been described hereinabove by way of specific embodiments thereof, it can be modified, without departing from the spirit and nature of the subject invention as defined in the appended claims.

### REFERENCES

1. Ahmed F E (2002) Molecular techniques for studying gene expression in carcinogenesis. J Environ Sci Health Part C Environ Carcinog Ecotoxicol Rev 20, 77-116.
2. Ahlgren S C, Thakur V, Bronner-Fraser M (2002) Sonic hedgehog rescues cranial neural crest, from cell death induced by ethanol exposure. Proc Natl Acad Sci U S A. 99(16):10476-81.
3. Ausubel et al. (1994) Current protocols in Molecular Biology. Wiley, N.Y.
4. Birk O S, Casiano D E, Wassif C A, Cogliati T, Zhao L, Zhao Y, Grinberg A, Huang S, Kreidberg J A, Parker K L, Porter F D, Westphal H (2000) The LIM homeobox gene Lhx9 is essential for mouse gonad formation. Nature 403(6772):909-13.
5. Boyer A, Lussier J G, Sinclair A H, McClive P J, Silversides D W (2004) Pre-Sertoli Specific Gene Expression Profiling Reveals Differential Expression of Ppt1 and Brd3 Genes Within the Mouse Genital Ridge at the Time of Sex Determination. Biol Reprod 71(3):820-827.
6. Boyer A, Doman S, Daneau I, Lussier J, Silversides D W (2002a) Conservation of the function of DMRT1 regulatory sequences in mammalian sex differentiation. Genesis 34:236-243.
7. Boyer A, Silversides D W (2002b) Combining transgenic mouse models and suppression-subtractive hybridization techniques to identify differentially expressed genes in pre-Sertoli cells. Hawaii, March (abstract).
8. Bronner-Fraser M (2002) Molecular analysis of neural crest formation. J Physiol Paris 96(1-2):3-8.
9. Buchstaller J, Sommer L, Bodmer M, Hoffmann R, Suter U, Mantei N (2004) Efficient isolation and gene expression profiling of small numbers of neural crest stem cells and developing Schwann cells. Journal of Neuroscience 24(10):2357-2365.
10. Chen Z, Karaplis A C, Ackerman S L, Pogribny I P, Melnyk S, Lussier-Cacan S, Chen M F, Pai A, John S W, Smith R S, Bottiglieri T, Bagley P, Selhub J, Rudnicki M A, James S J, Rozen R (2001) Mice deficient in methylenetetrahydrofolate reductase exhibit hyperhomocysteinemia and decreased methylation capacity, with neuropathology and aortic lipid deposition. Hum Mol Genet. 10(5):433-43.
11. Daneau I, Pilon N, Boyer A, Behdjani R, Overbeek P A, Viger R, Lussier J, Silversides D W (2002) The porcine SRY promoter is transactivated within a male genital ridge environment. Genesis 33(4):170-180.
12. Ding G, and Cantor C R (2004) Quantitative analysis of nucleic acids—the last few years of progress. J Biochem Biol 37, 1-10.
13. Eriksson B, Bergqvist I, Eriksson M, Holmberg D (2000) Functional expression of Cre recombinase in sub-regions of mouse CNS and retina. FEBS Lett. 479(3):106-10.
14. Fernandes K J L et al. (2004) A dermal niche for multipotent adult skin-derived precursor cells. Nature Cell Biology. 6(11): 1082-1093.
15. Gammill L S, Bronner-Fraser M (2002) Genomic analysis of neural crest induction. Development 129(24):5731-41.
16. Hogan B, Beddington R, Constantini F, Lacy E (1994) Manipulating the mouse embryo: a laboratory manual, 2nd de. Cold Spring Harbor, N.Y.: Cold Spring harbor Laboratory Press.
17. Iyer A K, McCabe E R. Molecular mechanisms of DAX1 action. Mol Genet Metab. 2004 September-October; 83(1-2):60-73.
18. Jones E A, Crotty D, Kulesa P M, Waters C W, Baron M H, Fraser S E, Dickinson M E (2000) Dynamic in vivo imaging of postimplantation mammalian embryos using whole embryo culture. Genesis 34(4):228-35.
19. Kruger G M, Mosher J T, Bixby S, Joseph N, Iwashita T, Morrison S (2002) Neural crest stem cells persist in the adult gut but undergo changes in self-renewal, neuronal subtype potential, and factor responsiveness. Neuron 35:657-669.
20. Li J, Chen F, Epstein J A (2000) Neural crest expression of Cre recombinase directed by the proximal Pax3 promoter in transgenic mice. Genesis 26(2): 162-4.
21. Lim K C, Lakshmanan G, Crawford S E, Gu Y, Grosveld F, Engel J D (2000) Gata3 loss leads to embryonic lethality due to noradrenaline deficiency of the sympathetic nervous system. Nat Genet. 25(2):209-12.
22. Lopez M F, Pluskal M G (2003) Protein micro- and macroarrays: digitizing the proteome. J Chromatography B 787, 19-27.
23. Lyons P (2003) Advances in spotted microarray resources for expression profiling. Briefings in Functional Genomics and Proteomics 2, 21-30.
24. Machado A F, Martin L J, Collins M D (2001) Pax3 and the splotch mutations: structure, function, and relationship to teratogenesis, including gene-chemical interactions. Curr Pharm Des. 7(9):751-85.
25. Methot D, Reudelhuber T, Silversides D W (1995) Novel uses of tyrosinase minigene coinjection in transgenic mouse studies. Nucleic Acids Research 23(22):4551-4556.
26. Molkentin J D (2000) The zinc finger-containing transcription factors GATA-4,-5, and -6. Ubiquitously expressed regulators of tissue-specific gene expression. J Biol Chem. 275(50):38949-52.
27. Morrish B C, Sinclair A H. Vertebrate sex determination: many means to an end. Reproduction. 2002 October; 124(4):447-57.
28. Murtaugh L C, Melton D A. Genes, signals, and lineages in pancreas development. Annu Rev Cell Dev Biol. 2003;19:71-89.
29. Patient R K, McGhee J D (2002) The GATA family (vertebrates and invertebrates). Curr Opin Genet Dev. 12(4):416-22.
30. Pollock J D (2002) Gene expression profiling: methodological challenges, results, and prospects for addiction research. Chemistry and Physics of Lipids 121, 241-256.
31. Retaux S, Rogard M, Bach I, Failli V, Besson M J (1999) Lhx9: a novel LIM-homeodomain gene expressed in the developing forebrain. J Neurosci. 19(2):783-93.
32. Robert N M, Tremblay J J, Viger R S (2002) Friend of GATA (FOG)-1 and FOG-2 differentially repress the GATA-dependent activity of multiple gonadal promoters. Endocrinology 143(10):3963-73.
33. Ruest L B, Dager M, Yanagisawa H, Charite J, Hammer R E, Olson E N, Yanagisawa M, Clouthier D E (2003) Dhand-cre transgenic mice reveal specific potential functions of dHAND during craniofacial development. Dev Biol. 257(2):263-77.
34. Sambrook et al. (1989) Molecular Cloning—A Laboratory Manual. Cold Spring Harbor Laboratories.
35. Scheel J, Von Brevern M C, Horlein A, Fisher A, Schneider A, Bach A (2002) Yellow pages to the transcriptome. Pharmacogenomics 3, 791-807.
36. Sieber-Blum M et al. (2004a) Pluripotent Neural Crest Stem Cells in the Adult Hair Follicle. Developmental Dynamics. 231: 258-269.
37. Sieber-Blum M, Grim M (2004b) The Adult Hair Follicle: Cradle for Pluripotent Neural Crest Stem Cells. Birth Defects Research (Part C). 72: 162-172.
38. Taketo M et al. (1991) FVB/N: an inbred mouse strain preferable for transgenic analyses. PNAS USA 88,2065-2069.
39. Tremblay J J, Robert N M, Viger R S (2001) Modulation of endogenous GATA-4 activity reveals its dual contribution to Mullerian inhibiting substance gene transcription in Sertoli cells. Mol Endocrinol. 15(9):1636-50.
40. Tremblay J J, Viger R S (2001) GATA factors differentially activate multiple gonadal promoters through conserved GATA regulatory elements. Endocrinology 142(3):977-86.
41. Tremblay J J, Viger R S (2003) Transcription factor GATA-4 is activated by posphorylation of Serine 261 via the camp/protein kinase A signaling pathway in gonadal cells. JBC 278(24):22128-35.
42. Viger R S, Mertineit C, Trasler J M, Nemer M (1998) Transcription factor GATA-4 is expressed in a sexually dimorphic pattern during mouse gonadal development and is a potent activator of the Mullerian inhibiting substance promoter. Development 125(14):2665-75.
43. Wilson M E, Scheel D, German M S. Gene expression cascades in pancreatic development. Mech Dev. 2003 January; 120(1):65-80.

What is claimed is:

1. A vector comprising a promoter sequence driving the coding sequence of a visual marker protein, wherein the promoter sequence functions specifically in neural crest cells.

2. A vector as recited in claim 1 wherein said promoter sequence is a GATA4 promoter sequence.

3. A vector as recited in claim 2 wherein said promoter sequence is a rat GATA4 promoter sequence as set forth in SEQ ID NO: 1.

4. A vector as recited in claim 1 wherein said promoter sequence is a LHX9 promoter sequence.

5. A vector as recited in claim 4 wherein said promoter sequence is a human LHX9 promoter sequence as set forth in SEQ ID NO: 2.

6. A vector as recited in claim 1 wherein said promoter sequence is a WT-1 promoter sequence.

7. A vector as recited in claim 6 wherein said promoter sequence is a human WT-1 promoter sequence as set forth in SEQ ID NO: 3.

8. A vector as recited in claim 1 wherein said promoter sequence is a DAX1 promoter sequence.

9. A vector as recited in claim 8 wherein said promoter sequence is a pig DAX1 promoter sequence as set forth in SEQ ID NO: 4.

10. A vector as recited in claim 1 wherein said promoter sequence is a SRY promoter sequence.

11. A vector as recited in claim 10 wherein said promoter sequence is a pig SRY promoter sequence as set forth in SEQ ID NO: 5.

12. A vector as recited in claim 1, wherein the visual marker protein is a fluorescent protein.

13. A vector as recited in claim 12, wherein the fluorescent protein is selected from the groups consisting of Green fluorescent protein (GFP), Cyanin fluorescent protein (CyaninFP), Yellow fluorescent protein (YellowFP), Blue fluorescent protein (BlueFP) and Red fluorescent protein (RedFP).

14. A recombinant host cell comprising a vector as recited in claim 1.

15. A cell population comprising a cell as recited in claim 14.

16. A transgenic non human animal comprising a recombinant host cell as recited in claim 14.

17. A method of observing neural crest cells activity comprising generating a transgenic non human animal having cells which comprise a promoter sequence functioning specifically in neural crest cells, and wherein the promoter drives the expression of a visual marker protein, whereby expression of the marker protein in the transgenic non human animal denotes neural crest cells activity.

18. A method as in claim 17, wherein the promoter is selected from the group consisting of GATA4, LHX9, WT-1, DAX1 and SRY.

19. A method of isolating neural crest cells comprising:
producing a transgenic non-human animal as defined in claim 16; dissecting tissues known to contain neural crest cells, and isolating cells expressing the marker protein from said tissues, whereby cells expressing the marker protein in the dissected tissues are neural crest cells.

20. A cell isolated through the method of claim 19.

21. A cell population derived from a cell as recited in claim 20.

22. A method of identifying genes expressed in neural crest cells comprising assaying cells as recited in claim 20 for gene expression.

23. A method of isolating pancreatic cells comprising:
producing a transgenic non-human animal as defined in claim 16; isolating the region of the animal known to contain pancreatic tissue from the remainder of the animal to yield a dissected pancreatic region, and isolating cells expressing the marker protein from said dissected pancreatic region, whereby cells expressing the marker protein in the dissected pancreatic region are pancreatic cells.

24. A cell isolated through the method of claim 23.

25. A cell population derived from a cell as recited in claim 24.

26. A method of identifying genes expressed in pancreatic cells comprising assaying cells as recited in claim 25 for gene expression.

27. A method for identifying a biological agent which affects proliferation, differentiation or survival of neural crest cells or pancreatic cells, comprising:
(a) comparing the proliferation, differentiation or survival of a cell population as defined in claim 21 in the absence and in the presence of a candidate biological agent, wherein said candidate biological agent is selected when the proliferation, differentiation or survival of said cell population differs in the absence and in the presence of said candidate agent.

28. The method of claim 27, wherein step (a) comprises determining the effects of said candidate biological agent on the differentiation of said cell population.

29. The method of claim 27, further comprising the step of inducing differentiation of said cell population prior to performing step (a).

30. The method of claim 27, wherein step (a) comprises determining the effects of said candidate biological agent on the proliferation of said cell population.

31. The method of claim 27, wherein said candidate biological agent is a growth factor selected from the group consisting of FGF-1, FGF-2, EGF, EGF-like ligands, TGF.alpha., IGF-1, NGF, PDGF, TGFβs and bFGF.

32. The method of claim 27, wherein said cell population consists essentially of the progeny of a single of said fluorescent cells.
